(19) United States
(12) Patent Application Publication
(10) Pub. No.: US 2003/0166160 A1
(43) Pub. Date: Sep. 4, 2003

(54) Compounds and molecular complexes comprising multiple binding regions directed to transcytotic ligands

(76) Inventors: Stephen B. Hawley, San Diego, CA (US); Steven Chapin, San Diego, CA (US); Philip L. Sheridan, San Diego, CA (US); L. L. Houston, Del Mar, CA (US); Jacqueline M. Glynn, San Diego, CA (US)

Correspondence Address: Richard J. Warburg, FOLEY & LARDNER, P.O. Box 80278, San Diego, CA, 92138-0278, US

(21) Appl. No.: 09/949,039

(22) Filed: Sep. 6, 2001

(51) Int. Cl. C12Q 1/68; C07H 21/04; C12P 21/04; C12P 21/02; C12N 5/06; C07K 14/435

(52) U.S. Cl. 435/69.7; 435/320.1; 435/325; 530/350; 536/23.5; 435/6

(57) ABSTRACT

Disclosed herein are multimeric molecular complexes and compounds that are multivalent, i.e., they have two or more targeting elements directed to a ligand that confers paracellular transporting properties and/or transcytotic properties to complexes and compounds to which it is bound. The complexes and compounds have properties that are different from the properties of monomers, complexes and compounds having only one targeting element directed to a paracellular and/or transcytotic ligand. The complexes and compounds of the invention undergo endocytosis, transcytosis and exocytosis; following endocytosis, the complexes or compounds may be transported into the cytosol or an organelle of a cell. In polarized cells, transcytosis can proceed in a “forward” or “reverse” direction. Reverse transcytosis is used for the non-invasive delivery of biologically active agents from the lumen of, e.g., the gastrointestinal tract or the airways of lungs, to the circulatory system. The complexes and compounds are incorporated in various compositions and medical devices suitable for medicinal or veterinary use.

### FIELD OF THE INVENTION

The inventions disclosed herein relate to compositions that pass through cellular barriers to deliver compounds into, through and out of cells, and methods of producing and using such compositions.

### BACKGROUND OF THE INVENTION

The following description of the background of the invention is provided simply as an aid in understanding the invention and is not admitted to describe or constitute prior art to the invention.

Therapeutic drugs can be introduced into the body using a variety of formulations and by various of routes of administration. For many reasons, a preferred route of administration is one that is non-invasive, i.e, does not involve any physical damage to the body. Generally, physical damage of this type results from the use of a medical device, such as a needle, to penetrate or breach a dermal surface or other external surface of an animal. Invasive routes of administration include, for example, surgical implants and injections. Injections can be intravascular, intrathecal or subcutaneous, all of which have undesirable features. Non-invasive routes of administration include uptake from the gastrointestinal tract as well as non-invasive parenteral (i.e, other than gastrointestinal) routes such as, e.g., inhalation therapy.

Presently, there are few, if any, formulations for the administration of proteins, a relatively new type of therapeutic drug. This is especially true in the case of non-invasive routes of administration and formulations therefor. Despite the enormous potential of therapeutic proteins, the lack of compositions and methods for the non-invasive administration of proteins has, depending on the particular protein in question, limited or prevented the medical use thereof.

Compounds are trafficked into, out from and within a cell by various molecules. “Endocytosis” is a general term for the process of cellular internalization of molecules, i.e, processes in which cells takes in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another.

Active endocytosis, exocytosis and transcytosis typically involve or are mediated by receptors, molecules that are at least partially displayed on the surface of cells. Receptors have varying degrees of specificity; some are specific for a single molecule (e.g., a receptor specific for epidermal growth factor; or a receptor that specifically recognizes Ca⁺⁺); some are semi-specific (e.g., a receptor that mediates the cellular internalization of many members of a family of cellular growth factors, or a receptor that recognizes Ca⁺⁺, Mg⁺⁺ and Zn⁺⁺); or of limited specificity (e.g., a receptor that mediates the cellular internalization of any phosphorylated protein, or a receptor that recognizes any divalent cation). Other types of molecules that can cause or influence the entry of molecules into cells include, e.g., cellular pores, pumps, and coated pits. Pores such as gated channels and ionophores form a channel that extends through the cellular membrane and through which certain molecules can pass. Cellular pumps exchange one type of molecule within a cell for another type of molecule in the cell's environment. Coated pits are depressions in the cellular surface that are “coated” with bristlelike structures and which condense to surround external molecules; the condensed coated pits then “pinch off” to form membrane-bound, coated vesicles within the cell.

Molecules that cause, influence or undergo endocytosis, exocytosis and/or transcytosis can do so constitutively, i.e, at all times, or regulated, for example, only under certain conditions or at specific times. Some such molecules can only mediate and/or undergo endocytosis, whereas some mediate and/or undergo transcytosis as well as endocytosis. Moreover, some such molecules are present in all or most cells (i.e, are ubiquitous), or are present mostly or only in certain tissues (i.e, are tissue-specific) or particular cell types.

The lack of compositions and methods causing, enhancing, mediating or regulating the endocytosis of therapeutic, diagnostic or analytical compounds and compositions hinders or prevents various uses of such compounds. In particular, the full therapeutic potential of many compounds could be realized if they were taken up by cells lining the gastrointestinal tract, as one could then formulate pills or tablets for the administration of therapeutic agents to patients. Typically, pills and other formulations for the oral delivery, and suppositories for the rectal delivery, of therapeutic agents to the gastrointestinal tract result in better patient compliance, and less use of medical resources, as opposed to other delivery modalities such as, e.g., intravenous administration. Similarly, the therapeutic potential of many compounds could be realized if they were taken up by cells lining the respiratory tract, including the nasal cavity; cells lining the gastrointestinal tract; vaginal surfaces; on dermal surfaces; and ocular surfaces and buccal surfaces (see Sayani et al., Crit. Rev. Ther. Drug Carrier Systems 13:85-184, 1996). Attempts to develop oral delivery formulations for proteins are discussed by Wang (J. Drug Targeting 4:195-232, 1996), Sinko et al. (Pharm. Res. 16:527, 1999) and Stoll et al. (J. Controlled Release 64:217-228, 2000).

In addition to the need for compositions and methods for the entry of biologically active molecules into cells, there is a further need for compositions and methods for causing, enhancing, mediating or regulating, or that control the direction of, transcytosis. Transcytosis is the general term given for processes whereby molecules, including biologically active molecules, move from one side or surface of a cell to another.

Furthermore, degradation and inefficient absorption of compounds delivered by conventional means further reduces the efficacy of those compounds. The ability to utilize alternative delivery pathways, target particular cells and tissues for delivery, improve the retention and absorption of compounds to be delivered, and protect the effective compound during delivery would be of significant import to the pharmaceutical and biopharmaceutical industries.

The above limitations vis-à-vis cellular transport of molecules are present both in vitro (e.g., in cellular cultures) and in vivo (e.g., in animals). Such limitations prevent or limit the therapeutic, diagnostic and/or analytical uses as of various compounds and compositions in an animal, including a mammal which may be a human. Such uses are described herein.

One example of a molecule that undergoes or mediates endocytosis, exocytosis as well as forward and reverse transcytosis is the polymeric immunoglobulin receptor (pIgR). The following information regarding pIgR is provided to assist in understanding the background of the invention.

Typically, pIgR molecules are displayed on epithelial cells. Epithelial cells line the interior of organs that have enclosed, semi-enclosed or compartmentalized spaces. The interior (e.g., canals, ducts, cavities, etc.) of such organs is generically referred to as the lumen. The lumen of a particular organ may have a specific name, e.g., the gastrointestinal lumen, pulmonary lumen, nasal lumen, nasopharyngeal lumen, pharyngeal lumen, buccal (within the mouth) lumen, sublingual (under the tongue) lumen, vaginal lumen, urogenital lumen, ocular lumen, or tympanic lumen. See, for example, Fahey et al., Immunol. Invest. 27:167-180, 1998; Brandtzaeg, J. Reprod. Immunol. 36:23-50, 1997; Kaushic et al., Biol. Reprod. 57:958-966, 1997; Richardson et al., J. Reprod. Immunol. 33:95-112; Kaushic et al., Endocrinology 136:2836-2844, 1995. Some of these might also be characterized as surfaces, e.g., the ocular surface.

Adjacent epithelial cells are connected by tight junctions. Disruption of tight junctions allows agents within the lumen, which often has an opening to the external environment of an animal, to penetrate into the body. Although such agents might include therapeutic agents, entry into the body via a disrupted tight junction is not specific; undesirable agents (e.g., bacteria, viruses, toxins and the like) will also be taken into the body. Due to this lack of specificity, as well as other factors, disruption of tight junctions for drug delivery purposes is generally not feasible and would, in any event, have many potential undesirable side effects.

Epithelial cells have two distinct surfaces: the apical side, which faces the lumen and is exposed to the aqueous or gaseous medium present therein; and an opposing basolateral (a.k.a. basal lateral) side that rests upon and is supported by an underlying basement membrane. The tight junctions between adjacent epithelial cells separate the apical and basolateral sides of an individual epithelial cell.

Epithelial cells are said to have polarity, that is, they are capable of generating gradients between the compartments they separate (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999). This polarity reflects that fact that the cell has distinct plasma membrane domains (apical and basolateral) having distinct transport and permeability characteristics. For example, the apical side often contains microvilli for the adsorption of substances from the lumen, and, in ciliated cells, cilia are found on the apical membrane. As another example, the Na⁺/K⁺-ATPase pump is characteristically found only on the basolateral membrane.

FIG. 1 shows the pathways of cellular transport involving the pIgR protein, which undergoes or mediates endocytosis, exocytosis as well as forward and reverse transcytosis, in epithelial cells. Molecules of pIgR are typically displayed on the surfaces of epithelial cells and direct the trafficking of immunoglobulin (IgA) molecules. Other classes and species of immunoglobulins may also be trafficked. The right side of FIG. 1 illustrates the “forward” (i.e, basolateral to apical) transcytosis of pIgR molecules, whereas “reverse” (apical to basolateral) transcytosis is shown on the left side of the figure.

Forward transcytosis is the best characterized biological function of pIgR, and serves to convey protective antibodies (IgA and IgM immunoglobulins) from the circulatory system to the lumen of an organ. In forward transcytosis, pIgR molecules displayed on the basolateral side of the cell bind IgA molecules in the bloodstream, and pIgR:IgA complexes are then endocytosed, i.e, taken up into the cell and into a vesicle. The pIgR:IgA complexes are transported to the apical side of the cell, where they are displayed on the cell surface. Delivery of IgA into the lumen occurs when the pIgR portion of a pIgR:IgA complex is cleaved, i.e, undergo proteolysis. This event separates the pIgR molecule into two components: the “secretory component” (SC), which is released into the lumen, and which remains bound to IgA in order to protect IgA from degradation, and the “stalk,” which remains displayed, at least temporarily, on the apical surface of the cell.

Surprisingly, ligands bound to stalks displayed on the apical side of a cell can undergo reverse transcytosis, i.e, transcytosis in the opposite direction of forward transcytosis, i.e, from the apical side of a cell to its basolateral side. In reverse transcytosis, pIgR molecules or portions thereof move from the apical surfaces of cells that line the lumen of an organ to the basolateral surfaces of these cells. In theory, pIgR-mediated reverse transcytosis could be used to deliver agents from a lumen (e.g., the interior of the gut or the airways of the lung) to the circulatory system or some other interior system, organ, tissue, portion or fluid of the body including by way of non-limiting example the lymphatic system, the vitreous humor, etc. For example, as is shown in FIG. 1, a compound having an element that binds to a portion of pIgR that undergoes reverse transcytosis could, due to its association with the pIgR stalk, be carried to the basolateral side of a cell, where it would be contacted with and/or released into the bloodstream.

Evidence has been presented that forward transcytosis is mediated by a vesicular process (Apodaca et al., J. Cell Biol. 125:67-86, 1994; Mostov, Annu. Rev. Immunol. 12:63-84, 1994), although the process may vary between different cell types (Sarnataro et al., Detergent insoluble microdomains are not involved in transcytosis of polymeric Ig receptor in FRT and MDCK cells, Traffic 2000 October;1(10):794-802). Although not wishing to be bound by any particular theory, FIG. 1 shows a similar vesicular mediated transport mechanism for reverse transcytosis. FIG. 1 is not intended to imply that such a mechanism actually exists because evidence to this fact is not available; the vesicular nature of reverse transcytosis is only a hypothesis based on what is known about forward transcytosis.

The polyimmunoglobulin receptor (pIgR) is reviewed by Mostov and Kaetzel, Chapter 12 in: Mucosal Immunology, Academic Press, 1999, pages 181-211 (1999). Other reviews of pIgR, transcytosis and mucosal immunity include Apodaca et al., The polymeric immunoglobulin receptor. A model protein to study transcytosis, J Clin Invest 87:1877-82, 1991; Kaetzel, Polymeric Ig receptor: defender of the fort or Trojan horse? Curr Biol 11:R35-8, 2001; Mostov, Regulation of protein traffic in polarized epithelial cells Histol Histopathol 10:423-31, 1995; Mostov et al., Regulation of protein traffic in polarized epithelial cells, Bioessays 17:129-38, 1995; Mostov, Transepithelial transport of immunoglobulins, Annu Rev Immunol 12:63-84, 1994; Brandtzaeg et al., The B-cell system of human mucosae and exocrine glands, Immunol Rev 1999 October; 171: 45-87; and Norderhaug et al., Regulation of the formation and external transport of secretory immunoglobulins (Review), Crit Rev Immunol 1999;19(5-6):481-508.

Transgenic animals that have alterations in the structure or expression of pIgR have been described Shimada et al., Generation of polymeric immunoglobulin receptor-deficient mouse with marked reduction of secretory IgA, J Immunol 1999 Nov 15:163(10):5367-73; Johansen et al., Absence of epithelial immunoglobulin A transport, with increased mucosal leakiness, in polymeric immunoglobulin receptor/secretory component-deficient mice, J Exp Med 1999 October 4:190(7):915-22; and de Groot et al., Over-expression of the murine polymeric immunoglobulin receptor gene in the mammary gland of transgenic mice, Transgenic Res 1999 April;8(2):125-35, Erratum in: Transgenic Res 1999 August;8(4):319.

Phillips-Quagliata et al., The IgA/IgM receptor expressed on a murine B cell lymphoma is poly-Ig receptor, J Immunol Sep. 1, 2000;165(5):2544-55, is stated to demonstrate that T560, a mouse B lymphoma that originated in gut-associated lymphoid tissue, expresses pIgR.

The structures of pIgR and its Ig ligands have been investigated using molecular genetic techniques. Norderhaug et al., Domain deletions in the human polymeric Ig receptor disclose differences between its dimeric IgA and pentameric IgM interaction, Eur J Immunol 1999 October;29(10):3401-9; Crottet et al., Covalent homodimers of murine secretory component induced by epitope substitution unravel the capacity of the polymeric Ig receptor to dimerize noncovalently in the absence of IgA ligand, J Biol Chem Oct. 29, 1999;274(44):31445-55; Breitfeld et al., Deletions in the cytoplasmic domain of the polymeric immunoglobulin receptor differentially affect endocytotic rate and postendocytotic traffic, J Biol Chem Aug. 15, 1990;265(23):13750-7; Casanova et al., Phosphorylation of the polymeric immunoglobulin receptor required for its efficient transcytosis, Science May 11, 1990;248(4956):742-5

Singer et al., Dimerization of the polymeric immunolgobulin receptor controls its transcytotic trafficking, Mol Biol Cell 1998 April;9(4):901-15, is stated to demonstrate that binding of dimeric IgA to chimeric pIgR/TCR molecules induces its dimerization (TCR is an abbreviation for the T cell receptor). The cytoplasmic domain of the T cell receptor-zeta chain was used as an indicator of receptor oligomerization to show that a pIgR:zeta chimeric receptor expressed in Jurkat cells initiates a zeta-specific signal transduction cascade when exposed to dimeric or tetrameric IgA, but not when exposed to monomeric IgA.

Eckman et al., Am J Respir Cell Mol Biol 1999 August;21(2):246-52, is stated to disclose a fusion protein consisting of a sFv directed to the secretory component (SC) of human pIgR and an human alpha-(1)-antitrypsin. Ferkol et al., Am. J. Respir. Crit. Care Med. 161:944-951, 2000, is stated to describe the basolateral-to-apical transport of the fusion protein of Eckman et al. across in vitro model systems of polarized respiratory epithelial cells. Gupta et al., Gene Ther 8:586-92, 2001, is stated to disclose the use of a single-chain antibody directed to the secretory component (SC) of human pIgR to deliver reporter genes to epithelial cells in vitro. The sFv is stated to be conjugated to polylysine using the cross-linker SPDP.

Zhang et al., Cell 102:827-837, 2000, states that pIgR translocates, Streptococcus pneumoniae across nasopharyngeal epithelial cells. The bacterial translocation is reported to occur in the apical to basolateral (reverse) direction.

Pilett et al., Reduced epithelial expression of secretory component in small airways correlates with airflow obstruction in chronic obstructive pulmonary disease, Am J Respir Crit Care Med 2001 January;163(1):185-94, is stated demonstrate that reduced expression of SC in airway epithelium is associated with airflow obstruction and neutrophil infiltration in severe chronic obstructive pulmonary disease.

U.S. Pat. No. 5,484,707 to Goldblum et al. is drawn to methods for monitoring organ rejection in an animal based on the concentration of the free secretory component of (SC) pIgR.

U.S. Pat. Nos. 6,020,161, 6,114,515, and 6,232,441, all to Wu et al., are stated to be drawn to pIgR polypeptides and polynucleotides that encode pIgR and pIgR-like polypeptides.

PCT patent applications WO 98/30592 and WO 99/20310, both to Hein et al., and U.S. Pat. No. 6,045,774 to Hiatt et al., are drawn to synthetic proteins that mimic IgA molecules and are thus associated with the proteolytically generated secretory component (SC) of pIgR.

U.S. Pat. No. 6,072,041 to Davis et al. is drawn to fusion proteins that are directed to the secretory component of pIgR. The compositions of Davis et al. are stated to be transported specifically from the basolateral surface of epithelial cells to the apical surface.

U.S. Pat. No. 6,261,787 B1 to Davis et al. is drawn to bifunctional molecules comprising (1) a ligand directed to the secretory component of pIgR and (2) a non-protein therapeutic molecule. The bifunctional molecules are said to be transported specifically from the basolateral surface of an epithelial cell to the apical surface thereof.

PCT application No. WO 00/53623, published Sep. 14, 2000, entitled “Bifunctional Molecules for Delivery of Therapeutics” by Davis, Pamela B., Ferkol Jr., Thomas W., and Eckman, Elizabeth, is stated to disclose bifunctional molecules that specifically bind secretory component (SC) of pIgR. The bifunctional molecules are said to be transported specifically from the basolateral surface of an epithelial cell to the apical surface thereof.

U.S. Pat. No. 6,042,833 to Mostov et al. is drawn to a method by which a ligand that binds to a portion of a pIgR molecule is thereby internalized into, or transported across, a cell expressing or displaying pIgR, Ser. No. 09/475,088 (attorney reference Nos. 2307E-067911US and 057220-0908) is a Divisional application of U.S. Pat. No. 6,042,833, that was filed Dec. 30, 1999. The corresponding PCT application was published as WO 97/46588, entitled “Cellular Internalization of pIgR Stalk and Associated Ligands” on Dec. 11, 1997.

U.S. patent application Ser. No. ______ (attorney docket No. 18062E-000900US) is entitled “Ligands Directed To The Non-Secretory Component, Non-Stalk Region of pIgR and Methods of Use Thereof” and was filed Mar. 26, 2001, by Mostov et al.

U.S. patent application Ser. No. 09/839,746 (attorney docket No.057220.0202), filed Apr. 19, 2001, entitled “Compositions Comprising Carriers and Transportable Complexes” by Houston, L. L., disclose various pharmaceutical compositions that may be applied to compositions and methods of the present invention.

U.S. patent application Serial No. 60/237,929 (attorney docket No. 057220.0301 {030854.0009.PRV1}) entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L., filed Oct. 2, 2000, is drawn to fusion proteins comprising targeting elements and biologically active polypeptides.

U.S. patent application Serial Nos. 60/248,478 and 60/248,819 (attorney docket No. 057220.0601 {030854.0009.PRV2}, and 057220.0602 {030854.0009.PRV3}, respectively), both entitled “Protein Conjugates of pIgR Ligands for the Delivery of Therapeutic and Diagnostic Proteins” by Houston, L. L., and Hawley, Stephen, filed Nov. 13, 2000 and Nov. 14, 2000 respectively, are drawn to protein conjugates comprising targeting elements and biologically active polypeptides.

U.S. patent application Serial No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., filed Feb. 2, 2001, is drawn to the identification and use of ligands and targeting elements directed to transcytotic and transepithelial molecules.

U.S. patent application Serial No. 60/267,601 (attorney docket No. 057220.0401) entitled “Polyspecific Binding Molecules Having a Polymeric Immunoglobulin Receptor Binding Region” by Houston, L. L., and Sheridan, Philip L., filed Feb. 9, 2001, is drawn to polyspecific compositions and compounds having (a) at least one ligand that specifically binds a pIgR molecule or the stalk molecule and (b) at least one ligand that (i) specifically binds a biologically active compound and/or (ii) is itself a biologically active compound.

U.S. patent application Serial No. 60/281,275 (attorney docket No. 057220.0501) entitled “Compositions and Methods for Transepithelial Transport of Membrane-Bounded Vesicles and Virions” by Sheridan, Philip L., and Houston, L. L., filed Apr. 3, 2001, is drawn to the use of targeting elements and ligands to deliver bounded compositions such as liposomes, virions, and the like.

U.S. patent application Ser. No. 09/898,503 (attorney docket No. 057220.1401) entitled “Compositions, Compounds And Methods For The Delivery Of Monoclonal Antibodies” by Hawley, Stephen, Chapin, Steve, and Houston, L. L., filed Jul. 2, 2001, is drawn to the use of targeting elements and ligands to deliver monoclonal antibodies and related compounds and compositions.

### SUMMARY OF THE INVENTION

The invention is drawn to molecular complexes and compounds comprising two or more targeting elements directed to a ligand that confers transcytotic and/or paracellular transporting properties to compounds and complexes to which it is bound. The invention provides compositions and methods for the transport of compounds into and/or through an epithelial barrier, including but not limited to the epithelial barriers that line the gastrointestinal tract and the lungs of an animal, which may be a mammal (the terms “animal” and “mammal” are as used in the art and encompass humans unless otherwise indicated).

In an epithelial layer, a grouping of epithelial cells is present in a stratum. An “epithelial barrier” is a surface comprising at least one epithelial layer that prevents or limits the passage of molecules from one side of the barrier to another. The epithelial layer or barrier may be found lining the lumen of an organ. A “lumen” is a canal, duct, cavity or other internal space of an organ. By way of non-limiting example, a lumen may be the gastrointestinal lumen, the pulmonary lumen, the nasal lumen, a nasopharyngeal lumen, a pharyngeal lumen, a buccal lumen, a sublingual lumen, a vaginal lumen, a urogenital lumen, an ocular lumen, a tympanic lumen or an ocular surface.

The complexes or compounds may be further transported to the circulatory system or the lymphatic system, or may act locally, e.g., on cells underlying or otherwise positioned near epithelial cells. Thus, the invention provides for the delivery of complexes and compounds from, e.g., the lumen of the gastrointestinal tract, that of the airways of the lungs, the nasal or vaginal surfaces, on dermal surfaces, ocular surfaces and buccal surfaces to the circulatory or lymphatic systems. The invention thus provides for the delivery of complexes and compounds via oral ingestion or suppository, via a capsule, caplet, tablet, time released formulation or the like; and for their delivery via inhalation; and for their delivery to such surfaces via gels, sols, viscous solutions and suspensions, ointments and the like.

Molecular Complexes and Compounds

A “molecular complex” comprises at two distinct molecules that are associated with each other by noncovalent interactions. By “associated” it is meant that the molecules in a complex, or moieties in a compound, remain specifically bound to each other for a given purpose. In a compound of the invention, the targeting elements are covalently bonded to each other whereas, in a molecular complex of the invention, the targeting elements are non-covalently associated.

The molecular complexes and compounds of the invention are multivalent, i.e., they comprise multiple binding regions for a target molecule, in the present instance a ligand that confers transcytotic and/or paracellular transporting properties to complexes and compounds to which it is bound. The targeting elements in any given compound or complex may be identical, substantially identical or different from each other.

The term “ligand” encompasses any type of composition or compound that is capable of binding to a molecular target. A “targeting element” is a compound or a moiety that is comprised within, respectively, a composition or compound, that is capable of binding to a molecular target. A compound or composition comprising a targeting element directed to (capable of specifically binding) a molecular target is a ligand of that target. It should be noted however, that a targeting element is itself a ligand when it exists in a free form that is not comprised within a composition or compound.

A preferred ligand that confers transcytotic and/or paracellular transporting properties to compounds and complexes to which it is bound is the stalk of the polyimmunoglobulin receptor (pIgR) and other defined regions of pIgR as set forth herein.

A “target molecule” or “molecular target” is a compound, a complex of two or more compounds, a moiety (a portion of a compound), or an interface formed between two or more compounds, to which a targeting element is directed. The compound, complex, moiety or interface may be known and have characterized structures, or may be of unknown structure whose presence may be inferred from detectable properties or activities. In some aspects of the invention, molecular targets include, by way of non-limiting example, pIgR, the secretory component of pIgR, the stalk of pIgR, a region of pIgR or the pIgR stalk that is defined by a specific amino acid sequence, or a domain of a pIgR molecule. A preferred domain for some aspects of the invention is domain 6, including but not limited to domain 6 sequences derived from a variety of animal species and hybrid domain 6 sequences formed from the combination of sequences of two or more different domain 6 sequences.

Transcytotic and Other Properties

Compounds are trafficked into, out from and within a cell by various processes. “Endocytosis” is a general term for the process of cellular internalization of molecules, i.e, processes in which cells takes in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another. As is explained in more detailed herein, epithelial cells are polarized, having two distinct surfaces, an apical surface and a basolateral surface. In epithelial cells, transcytosis may be in the “forward” (i.e, basolateral to apical) or “reverse” (apical to basolateral) direction (FIG. 1).

A “transcytotic property” is an attribute that causes, promotes or enhances endocytosis, exocytosis, transcytosis and/or intracellular delivery. Transcytotic properties include, by way of non-limiting example, the ability to undergo a least one process selected from the group consisting of apical endocytosis, apical exocytosis, apical to basolateral transcytosis, basolateral endocytosis, basolateral exocytosis, basolateral to apical transcytosis, and intracellular delivery.

By “intracellular delivery,” it is meant that a complex or compound is delivered into, and remains inside, the interior of a cell, whether within the cytosol or an organelle. In a related aspect, the compositions and compounds of the invention include an organelle-targeting sequence for transport to selected organelles. An “organelle” is a subcellular component that carries out one or more specific biological and/or biochemical functions. An “organelle-targeting sequence” is an amino acid sequence that mediates the delivery of a complex or compound having the organelle targeting sequence to an organelle of interest such as, e.g., a mitochondrion, the endoplasmic reticulum, the Golgi apparatus, lysosomes, peroxisomes, endosomes, the cell membrane or any membrane contained within a cell, the nucleus, or the nucleolus.

A “paracellular transporting property” is an attribute that causes, promotes paracellular transport including, by way of non-limiting example, transport through the tight gap junctions found in epithelial cell layers.

The multivalent complexes and compounds of the invention may have transcytotic, paracellular transporting or other properties that are enhanced relative to a corresponding monovalent complex or compound, or as compared to a corresponding multivalent complex or compound having a valency that is less than the complex or compound to which it is being compared.

By “enhanced” it is meant that one or more desirable attributes, including but not limited to transcytotic and paracellular transportation properties, is augmented, improved or introduced into a complex or compound. By way of non-limiting example, enhanced transcytotic properties include an increase in the relative rate of one or more processes such as of endocytosis, transcytosis or exocytosis; an increased range of recognition, or a higher degree of specificity, for particular types and species of pIgR and stalk molecules; or the ability to transcytose compounds of a larger molecular weight and/or a different composition. Similarly, enhanced properties of paracellular transport include but are not limited to an increase in the relative rate of transport; or the ability to transport compounds of a larger molecular weight.

The “relative rate” of a multimeric compound or complex of the invention refers to the number of molecules of a multimer undergoing a given process (endocytosis, transcytosis, paracellular transport, etc.) over a set period of time compared to the number of molecules of a comparable monomer undergoing the same process over the same period of time. Rates may also be expressed in absolute terms, e.g., x moles of molecules per nanosecond. Similarly, other properties of complexes and compounds may be measured in absolute or relative terms.

An enhanced property may also be a preference for reverse transcytosis (apical to basolateral transcytosis) as compared to forward (basolateral to apical) transcytosis. A preference for reverse trancytosis is desirable in aspects of the invention where delivery of complex and compounds from the lumen of an organ to the circulatory system is the desired goal.

Other properties that may be enhanced in the complexes and compounds of the invention include, by way of non-limiting example, increased stability of complexes and compounds in vitro or in vivo; increased yield or improved purity of complexes and compounds, particularly as produced by recombinant DNA expression systems; removal or reduction of one or more undesirable properties, e.g., undesired side effects; and the like.

Biologically Active Moieties and Molecules

Biologically active moieties and molecular are present in certain apsects of the invention. The term “biologically active” (synonymous with “bioactive”) indicates that a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that stimulates or causes an immunreactive response; one that impacts a biological process in an animal; one that impacts a biological process in a pathogen or parasite; one that generates or causes to be generated a detectable signal; and the like. Biologically active compositions, complexes or compounds may be used in therapeutic, prophylactic and diagnostic methods and compositions. Biologically active compositions, complexes or compounds act to cause or stimulate a desired effect upon an animal. Non-limiting examples of desired effects include, for example, preventing, treating or curing a disease or condition in an animal suffering therefrom; limiting the growth of or killing a pathogen in an animal infected thereby; augmenting the phenotype or genotype of an animal; stimulating a prophylactic immunoreactive response in an animal; or diagnosing a disease or disorder in an animal.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasite; or of cells, tissues or organs of an animal that have abnormal growth or biochemical characteristics, such as cancer cells.

In the context of diagnostic applications of the invention, the term “biologically active” indicates that the composition, complex or compound can be used for in vivo or ex vivo diagnostic methods and in diagnostic compositions and kits. For diagnostic purposes, a preferred biologically active composition or compound is one that can be detected, typically (but not necessarily) by virtue of comprising a detectable polypeptide. Antibodies to an epitope found on composition or compound may also be used for its detection.

In the context of prophylactic applications of the invention, the term “biologically active” indicates that the composition or compound induces or stimluates an immunoreactive response. In some preferred embodiments, the immunoreactive response is designed to be prophylactic, i.e, prevents infection by a pathogen. In other preferred embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

It will be understood by those skilled in the art that a given composition, complex or compound may be biologically active in therapeutic, diagnostic and prophylactic applications. A composition, complex or compound that is described as being “biologically active in a cell” is one that has biological activity in vitro (i.e, in a cell culture) or in vivo (i.e, in the cells of an animal). A “biologically active component” of a composition or compound is a portion thereof that is biologically active once it is liberated from the composition or compound. It should be noted, however, that such a component may also be biologically active in the context of the composition or compound.

Specific examples of compositions, complexes and compounds that are not biologically active include elements that have no effect on biological functions but which are incorporated for ease of manipulation of the conjugate or member thereof such as, e.g., poly-(L)-lysine for the in vitro chemical conjugation of the composition or compound to another molecule; a polypeptide derived from a phage surface protein intended for compositions or compounds to be used in vitro in phage display libraries; or a composition or compound that serves as a carrier for another composition or compound such as, e.g., KLH (keyhole limpet hemocyanin), which serves as a carrier for immunogenic compositions or compounds; or the herein-disclosed “optional fusion protein elements.”

Structures of Multivalent Complexes and Compounds

In the present disclosure, bivalent dimers (complexes or compounds having two targeting elements) are used as representative examples of multivalent compounds and complexes, but are not intended to be limiting examples. Because they have only two targeting elements, dimers are, from a structural standpoint, the least complicated type of multimers, a term which includes in its broadest sense all multivalent compounds and complexes having two or more targeting elements. The multivalent compounds and complexes of the invention may have three targeting elements (trimers), four targeting elements (tetramers), etc. It should thus be understood that dimers are used herein as non-limiting examples to illustrate all multivalent compounds and complexes.

In the disclosure, a covalent linkage (chemical bond) is represented by “-”, and a non-covalent bond by “::” A dimer according to the invention has a pair of targeting elements, TE1 and TE2. A dimer may be a compound that includes the substructure TE1-TE2, or a molecular complex that includes the substructure TE1::TE2.

The compounds and complexes of the disclosure may also comprise a biologically active molecule (BAM). The BAM may be covalently or non-covalently attached to one or more of the multiple targeting elements. In each complex or compound of the invention, targeting elements and biologically active molecules (BAM's) are independently selected as a compound (e.g., a small molecule, a nucleic acid or a polypeptide) and as moieties, ligands, compounds or complexes.

In the case of dimers of the invention, compounds comprising a single BAM have structures such as:

TE1-TE2-BAM;

TE1-BAM-TE2; and

BAM-TE1-TE2,

In the case of molecular complexes that comprise a BAM and are dimers, at least one of the targeting elements present therein are non-covalently bonded to each other, although a targeting element may be covalently bonded to the BAM; or the two targeting elements may be covalently bonded to each other, but non-covalently associated with the BAM. That is, dimeric molecular complexes of the invention have structures such as:

TE1::TE2::BAM;

TE1::TE2-BAM; and

TE1-TE2::BAM.

The molecular complexes of the invention may comprise one or more compounds of the invention. For example, in the case of “TE1-TE2::BAM”, “TE1-TE2” represents a dimeric compound that is part of a dimeric complex. A molecular complex that comprises a dimeric compound of the invention is a dimeric complex, but not all dimeric complexes comprise a dimeric compound (TE1::TE2::BAM being an example of the latter instance).

In a compound of the invention, the biologically active moiety and the two or more targeting elements may all be polypeptides. In a compound of the invention where the biologically active moiety and the targeting elements are all polypeptides, the compounds may be recombinantly-produced fusion proteins, or protein conjugates produced in part by in vitro chemical conjugation.

Aspects of the Invention

In one aspect, the invention provides multivalent molecular complexes that are (a) a molecular complex that comprises at least two separate compounds, each compound comprising at least one targeting element directed to a ligand that confers transcytotic or paracellular transporting properties to a molecular complex specifically bound to the ligand; or (b) a molecular complex that comprises at least two separate compounds, at least one of the compounds comprising at least two targeting elements. In the case of complexes comprising a biologically active molecule (BAM), non-limiting (dimeric) examples of type (a) complexes include “BAM::TE1::TE2”, “BAM-TE1::TE2” and “TE1::TE2-BAM”; a non-limiting (dimeric) example of type (b) complex has the structure “BAM::TE1-TE2”.

In another aspect, the invention provides multivalent compounds that comprise at least two targeting elements directed to a ligand that confers transcytotic or paracellular transporting properties to a compound specifically bound to the ligand. A non-limiting exemplary dimer of this aspect may be represented as “TE1-TE2”; when a BAM is present in the compound, an exemplary dimers may be represented as “TE1-TE2-BAM”, “BAM-TE1-TE2”, “TE1-BAM-TE2”, etc. Preferred polypeptidic compounds of the invention are fusion proteins and protein conjugates.

The multivalent complexes and compounds of the invention may be such that one or more of the transcytotic or paracellular transporting properties of the complex or compound are enhanced as compared to a complex or a compound having no more than one targeting element. Thus, in one aspect, the invention provides a (first) multimeric complex comprising n targeting elements directed to a ligand that confers transcytotic or paracellular transporting properties to a compound bound to said ligand, wherein one or more of the properties of the first multimeric molecular complex are enhanced as compared to those of a (second) compound having m targeting elements, wherein n and m are both whole integers, and n>m.

The multimeric molecular complexes and compounds of the invention may be such that one or more of the transcytotic or paracellular transporting properties of the complex are enhanced as compared to a complex having no more than 1 targeting element. Thus, in one aspect, the invention provides a (first) multimeric molecular complex comprising n targeting elements directed to a ligand that confers transcytotic or paracellular transporting properties to a complex bound to said ligand, wherein one or more of the properties of the first multimeric molecular complex are enhanced as compared to those of a (second) multimeric molecular complex having m targeting elements, wherein n and m are both whole integers, and n>m.

The targeting elements in any given compound or complex may be identical (T1=T2), substantially identical (T1≅T2) or different from each other. In this context “different” indicates that T1 and T2 are different in terms of chemical nature (i.e., T1 is a polypeptide and T2 is a nucleic acid); or in terms of their structures, even though they are of the same chemical nature (i.e., T1 and T2 are both polypeptides, but T1 is a single-chain antibody directed to a molecular target and T2 is an oligopeptide derived from a protein that is a natural ligand of the molecular target).

The biologically active moiety may be a targeting element directed to a second ligand, i.e., a ligand other than the ligand that confers transcytotic and/or paracellular transporting properties to compounds and complexes to which it is bound. For example, a multivalent complex or compound of the invention can be prepared wherein the biologically active moiety is a diagnostic or therapeutic monoclonal antibody directed to a pathogenic factor. Such complexes and compounds are said to be polyspecific.

In another aspect, the invention provides compositions comprising the complexes and compounds of the invention. The compositions of the invention may be formulated for therapeutic, diagnostic, prophylactic, research or other applications and uses.

In another aspect, the invention provides protein conjugates that comprise one or more targeting elements directed to a ligand that confers transcytotic or paracellular transporting properties to a compound specifically bound to the ligand and a biologically active calcitonin polypeptide. By “biologically active calcitonin polypeptide” it is meant calcitonin proteins and polypeptide derivatives thereof that retain at least one biological or biochemical activity of calcitonin. Preferred calcitonin polypeptides include human calcitonin (hClacitonin) and salmon calcitonin (sCalcitonin).

In another aspect, the invention provides pharmaceutical compositions and medical devices that include the compositions, complexes and compounds of the invention. A related aspect of the invention is drawn to delivering a bioactive molecule or moiety to an organism or cells, tissues or organs derived therefrom. In another related aspect, the invention provides methods of providing therapy using the pharmaceutical compositions and medical devices of the invention.

In another aspect, the invention provides methods of providing therapy to an animal suffering from a disease, comprising administering to the animal patient a therapeutically effective amount of one or more of the compositions, compounds or pharmaceutical compositions of the invention. The disease may be an inflammatory disease, such as, by way of non-limiting example, colitis; ulcerative colitis; diverticulitis; Crohn's disease; gastroenteritis; inflammatory bowel disease; bowel surgery ulceration of the duodenum, a mucosal villous disease including but not limited to coeliac disease, past infective villous atrophy and short gut syndromes; an apoptosis mediated disease; an inflammatory disease; an autoimmune disease; a proliferative disorder; an infectious disease; a degenerative disease; a necrotic disease, asthma; allergic bronchopulmonary aspergillosis; hypersensitivity pneumonia, eosinophilic pneumonia; emphysema; bronchitis; allergic bronchitis bronchiectasis; cystic fibrosis; hypersensitivity pneumotitis; occupational asthma; sarcoid, reactive airway disease syndrome, interstitial lung disease, hyper-eosinophilic syndrome, parasitic lung disease; lung cancer and diabetes, consisting of rheumatoid arthritis, multiple sclerosis, graft-versus-host disease, diabetes mellitus, sarcoidosis, granulomatous colitis, systemic lupus erythematosus and osteoarthritis, pancreatitis, asthma, adult respiratory distress syndrome, glomeralonephritis, rheumatoid arthritis, systemic lupus erythematosus, scleroderma, chronic thyroiditis, Grave's disease, autoimmune gastritis, insulin-dependent diabetes mellitus (Type I), autoimmune hemolytic anemia, autoimmune neutropenia, thrombocytopenia, chronic active hepatitis, myasthenia gravis, psoriasis, graft vs. host disease, osteoporosis, multiple myeloma-related bone disorder, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke in a patient Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and ophthalmoplegia.

In another aspect, the invention provides medical devices and kits comprising the compositions, compounds and pharmaceutical compositions of the invention. A non-limiting example of a medical device is an inhaler which is used to deliver monoclonal antibodies via the pulmonary route in inhalation therapy. A non-limiting example of a medical kit is a kit for treating snakebites in situations where medical services are not immediately available (e.g., military applications, hiking first aid kits, and the like).

In another aspect, the invention provides diagnostic compositions and kits comprising the compositions, compounds and diagnostic compositions of the invention, and methods of use thereof. A diagnostic composition or compound of the invention further comprises at least one detectably labeled agent. The diagnostic compositions and kits of the invention are used for the in vivo detection of any marker associated with a particular disease, or for the detection of such markers in samples removed from an animal suspected of suffering from, or that is prone to, the disease. Those skilled in the art will be able to prepare and use the diagnostic compositions or compounds of the invention in a variety of formats, including automated and semi-automated assays, including high throughput assays.

The summary of the invention disclosed above is not limiting and other features and advantages of the invention will be apparent from the following detailed description of the preferred embodiments, as well as from the claims.

### BRIEF DESCRIPTION OF THE DRAWINGS

FIG. 1 shows forward and reverse transcytotic pathways of the polyimmunoglobulin receptor (pIgR) in epithelial cells.

FIG. 2 shows alignments of the amino acid sequences of pIgR homologs. Panel 2A, alignment of human, pig, cow, mouse, rat, possum and rabbit pIgR molecules, showing the relative positions of the leader sequence, the region of pIgR the secretory component that binds immunoglobulin (Ig), domains 1-6, and the transmembrane domain (arrows, border between domains); Panel 2B, alignment of human, simian (CynMonk) and rabbit stalk molecules.

FIG. 3 shows the nucleotide sequence of pSyn5AF (SEQ ID NO: 1), a plasmid that encodes single chain antibody sFv5AF. The emboldened nucleotide sequence indicates the reading frame (ATG, start codon; TAA, stop codon); boxed sequences indicate restriction enzyme sites (aagctt, Hind III site; gaattc, EcoRI site).

FIG. 4 shows the amino acid sequence (SEQ ID NO:2) of the secreted form of the sFv5AF encoded by pSyn5AF. Symbols: Pelb leader, a leader sequence that directs secretion from E. coli; FLAG, FLAG epitope; linker, amino acid sequence (GGGS)₃; myc, c-myc epitope; 6 HIS, 6×His tag; CDR, complementarity-determining region; FR, framework element; and the heavy and light chains of the scFv are indicated. The sequence of sFv5AF is identical to that of sFv5A with the exception that the 5th residue in the sFv sequence is glutamine (Q) in 5A and leucine (L) in 5AF. The amino-terminal Pelb leader sequence is MKYLLPTAAAGLLLLAAQPAMA, and the carboxy terminal sequence is AAAEQKLISEEDLNGAAHHHHHH.

FIG. 5 shows the amino acid sequence of the secreted form of the sFv5AF-Cys. The sFv5AF-Cys protein consists of, from an amino- to carboxy-terminal direcetion, a pelb leader (for secretion in E. coli), a FLAG epitope tag, a heavy chain variable region, a spacer sequence [GGGGS repeated three times, i.e., (G4S)₃], a light chain variable region, another (G4S)₃ linker, a cysteine residue (emboldened “C”) that has been introduced into the sFv relative to sFv5AF, a c-myc epitope tag, and a 6×His tag (for purification by Immobilized Metal-ion Affinity Chromatography, IMAC). The framework (FR) and complementarity-determining regions (CDR) of the heavy chain and light chain are indicated. The non-immunoglobulin regions (Pelb leader, FLAG epitope tag, linker (G4S)₃, c-myc tag and 6×His tag) are shaded. In addition to the FLAG tag, the amino acid sequence of sFv5AF differs from sFv5A in that the 5th residue in the sFv sequence is changed from a glutamine (Q) to a leucine (L) aminod acid residue.

FIG. 6 shows the strategy for cloning a rat pIgR cDNA.

FIG. 7 shows the strategy for cloning a mouse pIgR cDNA.

FIG. 8 shows the strategy for cloning a human pIgR cDNA.

FIG. 9 shows the chimeric rabbit/rat pIgR molecule. Panel 6A shows the structure of the chimeric pIgR. Panel 6B shows the amino acid sequence of the chimeric pIgR The transmembrane domain of the chimera is underlined. The rat portion of the molecule is emboldened. This segment consists largely of domain 5 plus most of the transmembrane domain. The cleavage site of the signal sequence is indicated by “●”.

FIG. 10 shows the amino acid sequences for various pIgR species encoded within GST-pIgR fusion proteins. Amino acids not contained within the pIgR protein are shown in bold and underlined. The most amino terminal amino acids in the sequences (GS) denote the amino acid residues glycine and serine residues contained at the carboxy terminus of the GST portion of the fusion protein. The carboxy termini of the fusion proteins contain additional amino acids not contained within the pIgR protein; in some cases these additional residues include a “His epitope tag” (HHHHHH). A consensus amino acid sequence for this part of the pIgR protein is shown below the sequences for cynomolgus, human, rat and rabbit sequences.

FIG. 11 shows the partial amino acid sequence of a bacterial adhesion protein, CbpA (SEQ ID NO:_). Emboldened and underlined amino acid sequences indicate amino acid sequences that bind, or contain an element that binds, pIgR.

FIG. 12 shows the transwell transcytosis assay system.

FIG. 13 shows the results of assays that compares the transcytosis of sFv5AF-Cys monomers and dimers, and demonstrates sFv5AF-mediated M1 antibody transcytosis.

FIG. 14 shows the time course of transcytosis of monovalent (monomers) and multivalent (dimers) sFv5 molecules.

FIG. 15 shows a nucleotide sequence that encodes the salmon calcitonin having the amino acid sequence shown in the figure.

FIG. 16 shows a nucleotide sequence that encodes the human calcitonin having the amino acid sequence shown in the figure.

FIG. 17 shows an amino acid sequence alignment for several representative calcitonin proteins from different species.

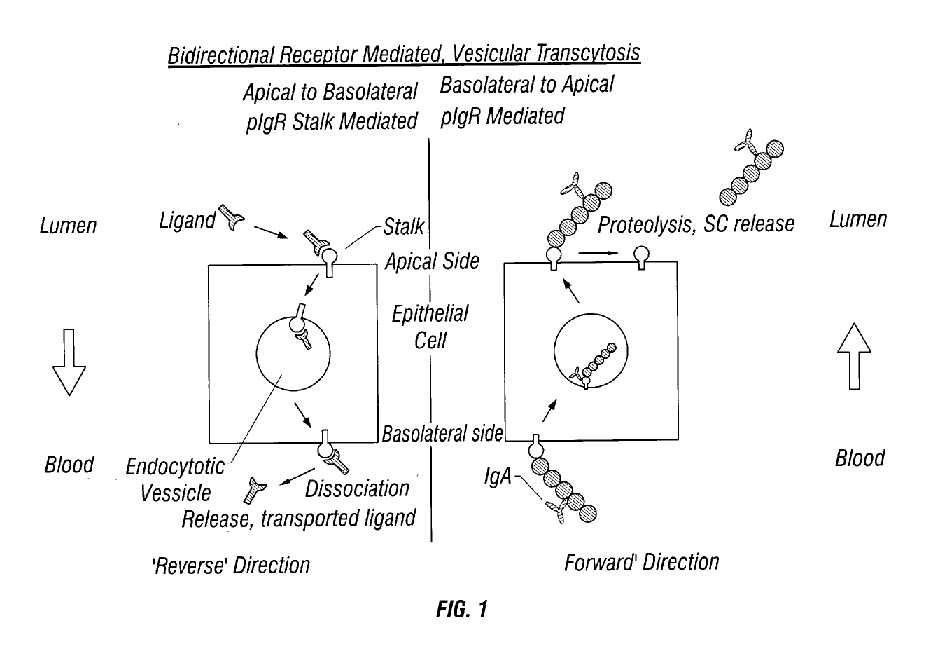

### DETAILED DESCRIPTION OF THE INVENTION

I. Structure and Function of pIgR

I.A. Structure of pIgR

A polyimmunoglobulin receptor (pIgR) molecule has several structurally and functionally distinct regions that are defined as follows. In the art, a pIgR molecule is generally described as consisting of two different, loosely defined regions called the “stalk” and the “secretory component” (SC). A pIgR molecule binds polymeric immunoglobulins (IgA or IgM) on the basolateral side, and then transports the immunoglobulin to the apical side. Proteolyic cleavage of pIgR takes place on the apical side of an epithelial cell between the SC and the stalk. The SC molecule is released from the cellular membrane and remains bound to and protects the immunoglobulins, whereas the stalk molecule remains bound to the cellular membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999).

Particularly preferred pIgR molecules are those described in U.S. Pat. No. 6,042,833, and the simian pIgR described in U.S. patent application Serial No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., which was filed on Feb. 2, 2001. However, it is understood that, in the context of this invention, pIgR also refers to any of that receptor's family or superfamily members, any homolog of those receptors identified in other organisms, any isoforms of these receptors, any pIgR-like molecule, as well as any fragments, derivatives, mutations, or other modifications expressed on or by cells such as those located in the respiratory tract, the gastrointestinal tract, the urinary and reproductive tracts, the nasal cavity, buccal cavity, ocular surfaces, dermal surfaces and any other mucosal epithelial cells. Preferred pIgR and pIgR-like proteins are those that direct the endocytosis or transcytosis of proteins into or across epithelial cells.

As used herein, the terms “secretory component” and “SC” refers to the smallest (shortest amino acid sequence) portion of an apical proteolyzed pIgR molecule that retains the ability to bind immunoglobulins (IgA and IgM). After proteolytic cleavage of pIgR, some amino acid residues remain associated with SC:immunoglobulin complexes but are eventually degaraded and/or removed from such complexes (Ahnen et al., J. Clin. Invest. 77:1841-1848, 1986). According to the definiton of the secretory component used herein, such amino acids are not part of the SC. In certain embodiments of the invention, pIgR-targeting elements that do not recognize or bind to the SC are preferred.

As used herein, the term “stalk” refers to a molecule having an amino acid sequence derived from a pIgR, wherein the stalk sequence does not comprise amino acid sequences derived from the SC. A stalk molecule comprises amino acid sequences that remain bound to the apical membrane following the apical proteolytic cleavage when such cleavage occurs and amino acid sequences required for such cleavage. Preferred stalk molecules confer one or more transcytotic properties to a ligand bound thereto. Most preferred are stalk molecules that confer the ability to undergo apical to basolateral transcytosis to a ligand bound thereto.

I.A.1. Protein Domains

Another way in which different portions of a pIgR molecule, and SC and stalk molecules derived therefrom, can be delineated is by reference to the domains thereof. A protein “domain” is a relatively small (i.e., <about 150 amino acids) globular unit that is part of a protein. A protein may comprise two or more domains that are linked by relatively flexible stretches of amino acids. In addition to having a semi-independent structure, a given domain may be largely or wholly responsible for carrying out functions that are normally carried out by the intact protein. In addition to domains that have been determined by in vitro manipulations of protein molecules, it is understood in the art that a “domain” may also have been identified in silico, i.e, by software designed to analyze the amino acid sequences encoded by a nucleic acid in order to predict the limits of domains. The latter type of domain is more accurately called a “predicted” or “putative” domain but, in the present disclosure, the term domain encompasses both known and predicted domains unless stated otherwise.

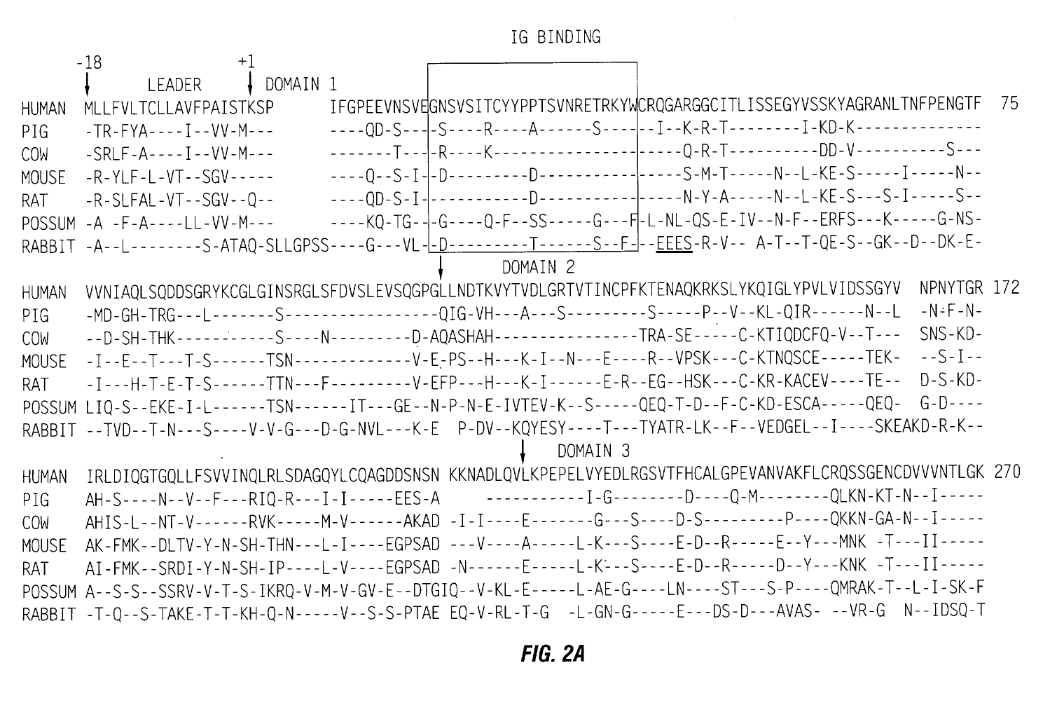

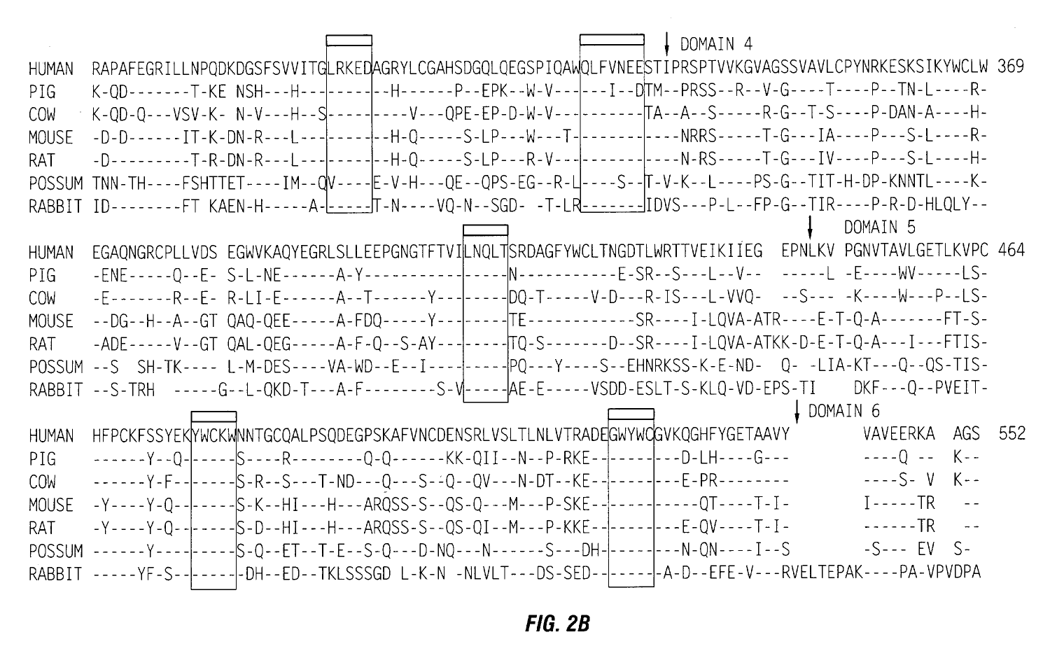

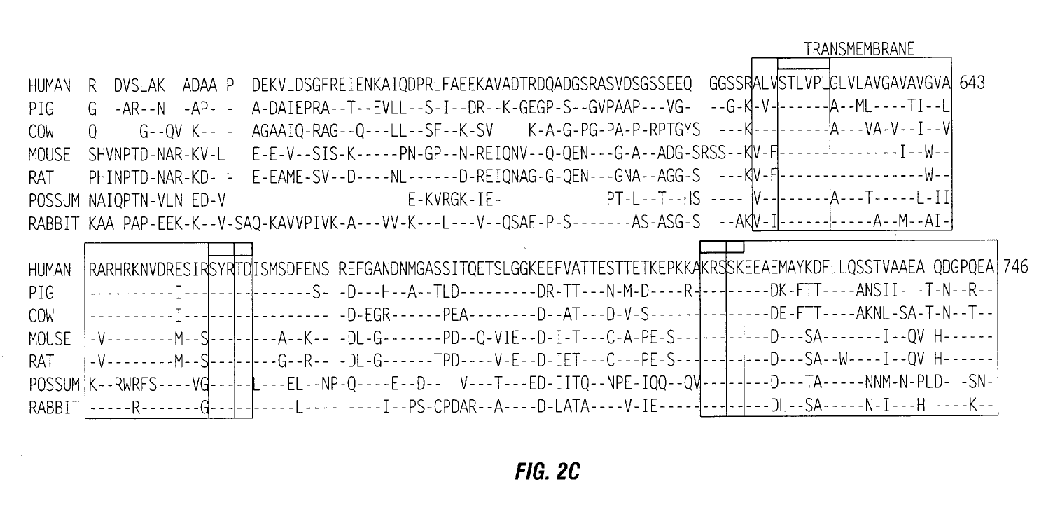

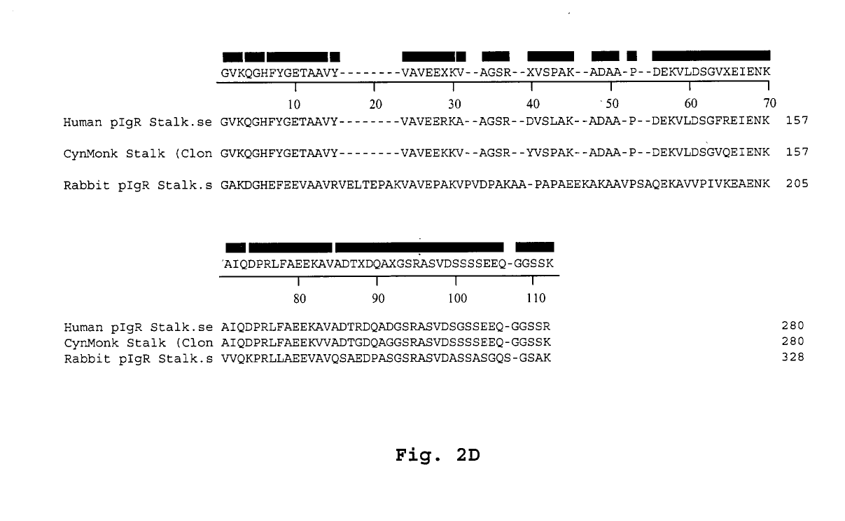

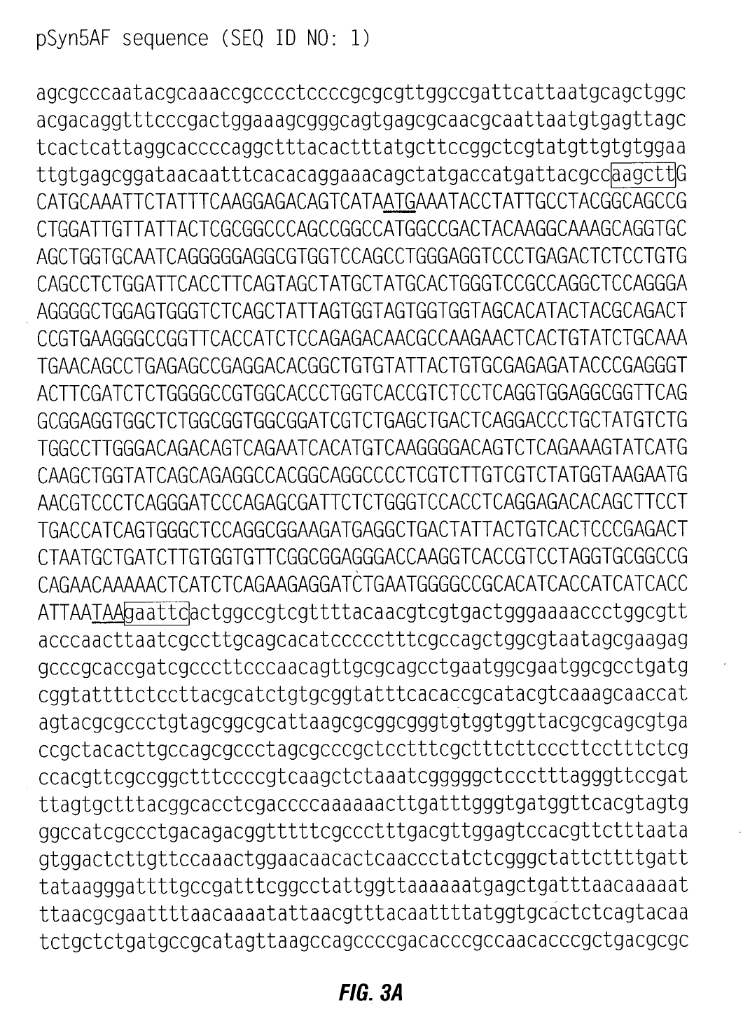

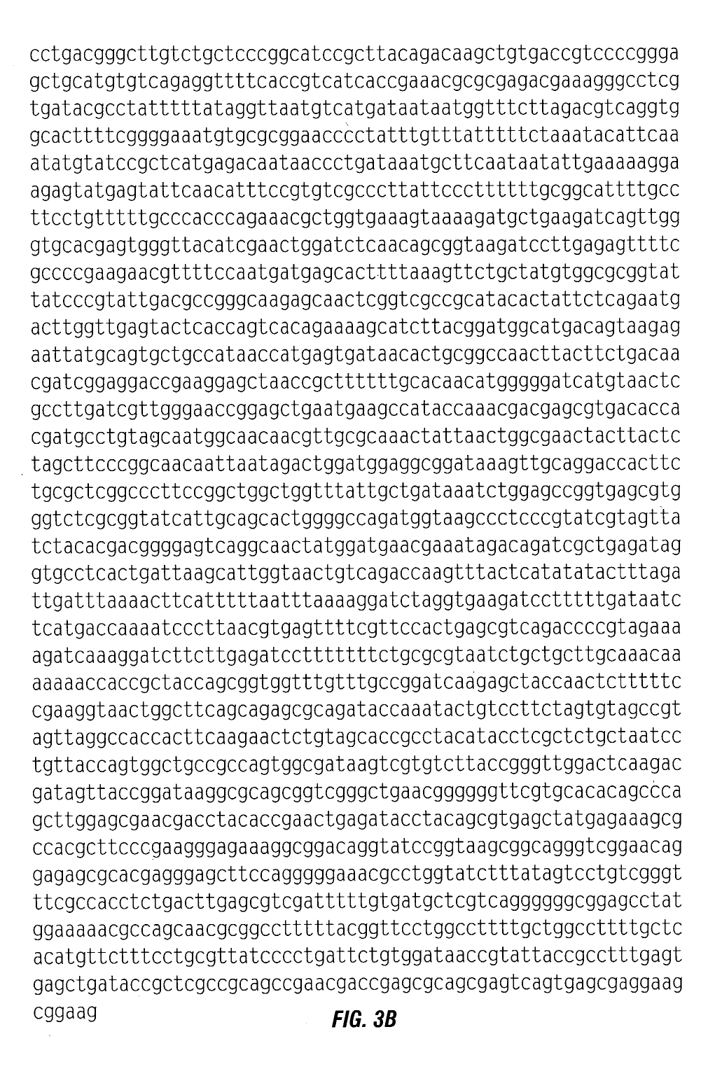

Domains of pIgR molecules include a leader sequence, extracellular domains 1 through 6, a transmembrane domain and an intracellular domain (see FIG. 2 herein and FIG. 3 of Piskurich et al., J. Immunol. 154:1735-1747, 1995). The intracellular domain contains signals for transcytosis and endocytosis. Domains of a pIgR molecule that are of particular interest in the present disclosure include but are not limited to domain 5, domain 6, the transmembrane domain and the intracellular domain. Preferred domains confer the ability to undergo apical to basolateral transcytosis to a ligand bound thereto.

I.A.2. Regions Defined by Conserved Sequences

Another way in which different portions of a pIgR molecule can be defined is by reference to amino acid sequences that are conserved between pIgR homologs (i.e., pIgR molecules isolated from non-human species; see below). Non-limiting examples of conserved amino acid sequences include those found in Table 1; see also FIG. 2. (For brevity's sake, the one letter abbreviations for amino acids is used in Table 1, but versions of sequences that employ the three letter amino acid designations may be found in the Sequence Listing; see also Table 2.)

TABLE 1
AMINO ACID SEQUENCES THAT ARE
CONSERVED IN PIGR HOMOLOGS

| Amino Acid Sequence | Position of Amino Acid Residues | |
|---|---|---|
| Conserved among | in Human pIgR Relative to | |
| pIgR Homologs | Amino Terminal Methionine* | SEQ ID NO: |
| LRKED | 297-301, inclusive | |
| QLFVNEE | 325-331, inclusive | |
| LNQLT | 410-414, inclusive | |
| YWCKW | 476-480, inclusive | |
| GWYWC | 522-526, inclusive | |
| STLVPL | 624-629, inclusive | |
| SYRTD | 658-662, inclusive | |
| KRSSK | 732-737, inclusive | |

*As described in FIG. 3 of Mostov and Kaetzel, Chapter 12 in: Mucosal Immunology, Academic Press, 1999, pages 181-211.

TABLE 2
ABBREVIATIONS FOR AMINO ACIDS

| Amino acid | Three-letter Abbreviation | One letter symbol |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 3
THE GENETIC CODE

| First | | | | | Third |
|---|---|---|---|---|---|
| position (5′ | Second Position | | | | position (3′ |
| end) | U | C | A | G | end) |
| U | Phe | Ser | Tyr | Cys | U |
| | Phe | Ser | Tyr | Cys | C |
| | Leu | Ser | Stop | Stop | A |
| | Leu | Ser | Stop | Trp | G |
| C | Leu | Pro | His | Arg | U |
| | Leu | Pro | His | Arg | C |
| | Leu | Pro | GIn | Arg | A |
| | Leu | Pro | GIn | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

Thus, for example, a specific internal portion of a given pIgR molecule might be defined as a region that has an amino-terminal border that has the amino acid sequence EKYWCKW and a carboxy-terminal border having the amino acid sequence side having the amino acid sequence DEGWYWCG. In human pIgR, the region so defined would be the amino acid sequence of residues 474 through 529. In the present invention, regions of any given pIgR molecule that are of particular interest include but are not limited to the regions described in Table 4 that are not conserved between pIgR homologs from different species:

TABLE 4
REGIONS OF PIGR AND STALK MOLECULES

| Region | Description |
|---|---|
| R1 | From KRSSK to the carboxy terminus, |
| R2a | From SYRTD to the carboxy terminus, |
| R2b | From SYRTD to KRSSK, |
| R3a | From STLVPL to the carboxy terminus, |
| R3b | From STLVPL to KRSSK, |
| R3c | From STLVPL to SYRTD, |
| R4a | From GWYWC to the carboxy terminus, |
| R4b | From GWYWC to KRSSK, |
| R4c | From GWYWC to SYRTD, |
| R4d | From GWYWC to STLVPL, |
| R5a | From YWCKW to the carboxy terminus, |
| R5b | From YWCKW to KRSSK, |
| R5c | From YWCKW to SYRTD, |
| R5d | From YWCKW to STLVPL, |
| R5e | From YWCKW to GWYWC, |
| R6a | From LNQLT to the carboxy terminus, |
| R6b | From LNQLT to KRSSK, |
| R6c | From LNQLT to SYRTD |
| R6d | From LNQLT to STLVPL, |
| R6e | From LNQLT to GWYWC, |
| R6f | From LNQLT to YWCKW, |
| R7a | From QLFVNEE to the carboxy terminus, |
| R7b | From QLFVNEE to KRSSK, |
| R7c | From QLFVNEE to SYRTD, |
| R7d | From LNQLT to STLVPL, |
| R7e | From QLFVNEE to GWYWC, |
| R7f | From QLFVNEE to YWCKW, |
| R7g | From QLFVNEE to LNQLT, |
| R8a | From LRKED to the carboxy terminus, |
| R8b | From LRKED to KRSSK, |
| R8c | From LRKED to SYRTD, |
| R8d | From LRKED to STLVPL, |
| R8e | From LRKED to GWYWC, |
| R8f | From LRKED to YWCKW, |
| R8g | From LRKED to LNQLT, and |
| R8h | From LRKED to QLFVNEE. |

Preferred regions confer the ability to undergo apical to basolateral transcytosis to a ligand bound thereto.

I.A.3. Target Molecules

Target molecules derived from a pIgR molecule, a secretory component (SC) molecule, or a stalk molecule, or to domains, conserved sequences, and defined regions thereof, are prepared as described herein and used as target molecules for the preparation of ligands and targeting elements of the invention. Preferred target molecules do not comprise amino acid sequences derived from the SC.

Target molecules may be chimeric, i.e., hybrid molecules derived from molecules from at least two different species. An example of a chimeric stalk target molecule is the rat/rabbit hybrid stalk molecule described in Example 2. A target molecule may also be a fusion protein, such as the domain 6-GST fusion proteins described in Example 3.

Preferred target molecules confer the ability to undergo apical to basolateral transcytosis to a ligand bound to a pIgR molecule or a stalk molecule, wherein the ligand does not bind specifically to an SC molecule. Other preferred target molecules comprise sequences from a stalk molecule. Target molecules may be produced using suitable techniques such as recombinant gene expression systems, chemical or enzymatic digestion of pIgR, SC or stalk molecules, or by in vitro synthesis of oligopeptides. Additionally or alternatively, target molecules may be genetically expressed in cells for techniques and experiments designed to assess transcytotic properties.

I.B. Proteins Related to pIgR

I.B.1. Homologs of pIgR

Homologs of pIgR are also within the scope of the invention. Homologs of pIgR are pIgR proteins from species other than Homo sapiens. By way of non-limiting example, pIgR proteins from various species include those from humans, the rat, mouse, rabbit, cow and possum (Table 5). See also FIG. 3 in Mostov and Kaetzel, Chapter 12, “Immunoglobulin Transport and the Polymeric Immunoglobulin Receptor” in Mucosal Immunity, Academic Press, 1999, pages 181-211; and Piskurich et al., J. Immunol. 154:1735-1747, 1995).

TABLE 5
PIGR AND PIGR-LIKE PROTEINS
FROM NON-HUMAN SPECIES

| ORGANISM | ACCESSION NUMBER(S) |
|---|---|
| Zebrafish | 9863256, 8713834, 8282255, & 7282118 |
| (Brachydanio rerio) | |
| Mouse (Mus musculus) | 8099664, 2804245, 6997240, 4585867, 4585866, |
| | 2688814, 2688813, 2688812, 2688811, 2688810, |
| | 2688809, 2688808, 2688807, 3097245, 3046754, |
| | 3046752, 3046751, 3046756, 3046755, 3046750, |
| | 3046748, 3046747 and 2247711 |
| Rat | 2222806, 475572, 475571, 473408, 603168 and |
| (Rattas norvegicus) | 603167 |
| Cow (Bos taurus) | 388279 |
| Possum (Trichosuras | 5305520, 5305518, 5305514 and 5305512 |
| vulpecula) | |

I.B.2. pIgR-like Proteins

Also within the scope of the invention are pIgR-like proteins. A “pIgR-like protein” is a protein that has an amino acid sequence having homolgy to a known pIgR protein. In many instances, the amino acid sequences of such pIgR-like molecules have been generated by the in silico translation of a nucleic acid, wherein the nucleotide sequence of the nucleic acid has been determined but is not known to encode a protein. By way of non-limiting example, pIgR-like proteins include PIGRL1 (U.S. Pat. No. 6,114,515); PIGR-1 (U.S. Pat. No. 6,232,441); a mouse gene having an exon similar to one of pIgR's (GenBank Accession No. 6826652); and human proteins translated in silico that have homology to pIgR proteins (GenBank Accession Nos. 1062747 and 1062741).

I.B.3. Substantially Identical and Homologous pIgR Molecules

As used herein, a “homolog” of a pIgR protein or a pIgR-like protein is an isoform or mutant of human pIgR, or a protein in a non-human species that either (i) is “identical” with or is “substantially identical” (determined as described below) to an amino acid sequence in human pIgR, or (ii) is encoded by a gene that is identical or substantially identical to the gene encoding human pIgR. Non-limiting examples of types of pIgR isoforms include isoforms of differing molecular weight that result from, e.g., alternate RNA splicing or proteolytic cleavage; and isoforms having different post-translational modifications, such as glycosylation; and the like.

Two amino acid sequences are said to be “identical” if the two sequences, when aligned with each other, are exactly the same with no gaps, substitutions, insertions or deletions. Two amino acid sequences are defined as being “substantially identical” if, when aligned with each other, (i) no more than 30%, preferably 20%, most preferably 15% or 10%, of the identities of the amino acid residues vary between the two sequences; (ii) the number of gaps between or insertions in, deletions of and subsitutions of, is no more than 10%, preferably 5%, of the number of amino acid residues that occur over the length of the shortest of two aligned sequences; or (iii) have only conservative amino acid substitutions (in one polypeptide as compared to another) that do not significantly affect the folding or activity of the polypeptide. Conservative amino acid substitutions are as described in Table 6). The entire amino acid sequence of two proteins may be substantially identical to one another, or sequences within proteins may demonstrate identity or substantial identity with sequences of similar length in other proteins. In either case, such proteins are substantially identical to each other. Typically, stretches of identical or substantially identical sequences occur over 5 to 25, preferably 6 to 15, and most preferably 7 to 10, nucleotides or amino acids.

TABLE 6
CONSERVATIVE AMINO ACID SUBSTITUTIONS

| Type of Amino | Groups of Amino Acids that Are Conservative |
|---|---|
| Acid Side Chain | Substitutions Other |
| Short side chain | Glycine, Alanine, Serine, Threonine and Methionine |
| Hydrophobic | Leucine, Isoleucine and Valine |
| Polar | Glutamine and Asparagine |
| Acidic | Glutamic Acid and Aspartic Acid |
| Basic | Arginine, Lysine and Histidine |
| Aromatic | Phenylalanine, Tryptophan and Tyrosine |

One indication that nucleotide sequences encoding pIgR proteins are substantially identical is if two nucleic acid molecules hybridize to each other under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 M at pH 7 and the temperature is at least about 60° C.

Another way by which it can be determined if two sequences are substantially identical is by using an appropriate algorithm to determine if the above-described critera for substantially identical sequences are met. Sequence comparisons between two (or more) polynucleotides or polypeptides are typically performed by algorithms such as, for example, the local homology algorithm of Smith and Waterman (Adv. Appl. Math. 2:482, 1981), by the homology aligment algorithm of Needleman and Wunsch (J. Mol. Biol. 48:443, 1970), by the search for similarity method of Pearson and Lipman (Proc. Natl. Acad. Sci. U.S.A. 85:2444, 1988), by computerized implementations of these algorithms (GAP, BESTFIT, FASTA, and TFASTA in the Wisconsin Genetics Software Package, Genetics Computer Group (GCG), 575 Science Dr., Madison, Wis.), or by visual inspection.

I.C. Binding and Transcytotic Assays

The ability of a pIgR ligand of the invention to bind different pIgR molecules, fragments and derivatives thereof, and to undergo endocytosis, transcytosis, and/or exocytosis is a desirable attribute of these proteins. The pIgR-binding capacity of fusion proteins are examined using the following techniques. Non-limiting examples of such assays include the following.

Cell lines that may be used in such assays are generally epithelial cells, particularly polarized cells having apical and basolateral surfaces. Such cells include those that naturally express pIgR or the pIgR stalk, preferably in response to factors and conditions that can be altered or manipulated, and cells that are transformed with nucleic acids encoding pIgR molecules, stalk molecules or target molecules prepared therefrom.

A non-limiting example of the former type of cells are epithelial cells isolated from human trachea, nasopharynx or bronchi. When grown on plastic, these primary cultures down-regulate expression of pIgR whereas, when grown on collagen-coated porous filters, the cultures produce pIgR (U.S. Pat. No. 6,261,787 B1 and Ferkol et al., Am. J. Respir. Crit. Care Med. 161:944-951, 2000).

Other non-limiting examples are T560, a mouse B lymphoma that originated in gut-associated lymphoid tissue that expresses pIgR (Phillips-Quagliata et al., The IgA/IgM receptor expressed on a murine B cell lymphoma is poly-Ig receptor, J Immunol Sep. 1, 2000;165(5):2544-55); and Fischer rat thyroid (FRT) cells (Samataro et al., Detergent insoluble microdomains are not involved in transcytosis of polymeric Ig receptor in FRT and MDCK cells, Traffic 2000 October;1(10):794-802; Aging effects on hepatic NADPH cytochrome P450 reductase, CYP2B1&2, and polymeric immunoglobulin receptor mRNAs in male Fischer 344 rats).

Cell lines that do not normally express the pIgR or the stalk, but which can be genetically transformed or transfected to express the pIgR, the stalk or target molecules include Madin-Darby canine kidney (MDCK) cells (as described throughout the specification and in Giffroy et al., Scand. J. Immunol. 53:56-64, 2001); chinese hamster ovary (CHO) cells (Asano et al., Molecular maturation and functional expression of mouse polymeric immunoglobulin receptor, J Immunol Methods May 1, 1998;214(1-2):131-9); endothelial cell lines such as ECV 304 (Su et al., Opposite sorting and transcytosis of the polymeric immunoglobulin receptor in transfected endothelial and epithelial cells, J Cell Sci 1998 May;111 (Pt 9):1197-206); and, particularly in instances where inhalation delivery of compounds is being tested, in cells from the 16HBEo bronchial cell line (Ferkol et al., Am. J. Crit. Care 16:944-951, 2000). Methods of transfecting cells in order to direct the expression of pIgR molecules therein are known in the art (Breitfeld et al., Methods in Cell Biology 32:329-337, 1989).

I.C.1. Ex Vivo Testing of Ligand Binding

The ex vivo pIgR binding capacity of a pIgR-targeted protein is assessed by measuring endocytosis or transcytosis of bound ligand in mammalian epithelial cells. Receptor-mediated endocytosis provides an efficient means of causing a cell to ingest material which binds to a cell surface receptor. (See Wu et al., J. Biol. Chem. 262:4429-4432, 1987; Wagner et al., Proc. Natl. Acad. Sci. USA 87:3410-3414, 1990, and published EPO patent application EP-A1 0388758). Any number of well known methods for assaying endocytosis may be used to assess binding. For example, binding, transcytosis, and internalization assays are described at length in Breiftifeld et al. (J. Cell Biol. 109:475-486, 1989).

Ligand-pIgR binding is measured by a variety of techniques known in the art, e.g., immunoassays and immunoprecipitation. By way of example, antibodies to the biologically active portion of a protein conjugate can be used to bind and precipitate detectably labeled pIgR or stalk molecules; the amount of labeled material thus precipitated corresponds to the degree of pIgR binding to a ligand such as, e.g., a protein conjugate having a pIgR-targeting element (see Tajima, J. Oral Sci. 42:27-31, 2000).

I.C.2. Apical Endocytosis

Apical endocytosis is conveniently measured by binding a ligand, such as sFv5 or a derivative thereof (see FIGS. 3 to 5), to a stalk molecule at the apical surface of transfected Madin-Darby canine kidney (MDCK) cells at 4° C., warming to 37° C. for brief periods (0-10 min), and cooling the cells back down to 4° C. Ligand molecules remaining on the surface are removed by stripping at pH 2.3. Intracellular ligand molecules are those that remain cell-associated after the stripping, while surface-bound ligand molecules are those removed by the acid wash. Controls for non-specific sticking include using molecules that are structurally related to the ligand but which do not bind to a pIgR or stalk molecule (e.g., an unrelated sFv in the case of sF5), and/or MDCK cells that are not transfected with genetic sequences encoding a pIgR molecule or a stalk molecule.

1.C.3. Apical to Basolateral Transcytosis

Apical to basolateral (“reverse”) transcytosis is assessed by allowing MDCK cells to bind the ligand at the apical surface at 4° C., followed by incubation at 37° C. for 0 to 240 min, and then measuring the amount of ligand delivered into the basolateral medium. This basolaterally-delivered ligand is compared to the sum of ligand that remains associated with the cells (intracellular or acid-stripped) and the ligand released back into the apical medium.

Alternatively, transcystosis is assessed by continuously exposing cells to the Fab in the apical medium and measuring accumulation of Fab in the basolateral medium. This method avoids cooling the cells. In both methods degradation of the ligand can be assessed by running aliquots of the transcytosed ligand on SDS-PAGE and probing a Western blot with antibodies that detect the ligand.

1.C.4. Basolateral Endocytosis

Basolateral endocytosis is assessed by methods such as those described by Tajima (J. Oral Sci. 42:27-31, 2000). Non-specific transport (e.g., fluid phase endocytosis and transcytosis, or paracellular leakage between cells) can be assessed as a control by using MDCK cells that are not transfected with a pIgR or stalk protein, and/or by the addition of antibody directed to the pIgR or stalk molecule.

I.C.5. In vivo Assays

In vivo transcytosis is assessed using pathogen-free experimental animals such as Sprague-Dawley rats. Detectably labeled ligand (e.g., a radioiodinated antibody) is administered into, e.g., the nares (the pair of openings of the nose or nasal cavity of a vertebrate) or the intestine (more details of these types of assays are provided herein in the Examples). As will be understood by those of skill in the art, a “detectable label” is a composition or moiety that is detectable by spectroscopic, photochemical, biochemical, immunochemical, electromagnetic, radiochemical, or chemical means such as fluorescence, chemifluoresence, or chemiluminescence, or any other appropriate method.

In vivo apical to basolateral (“reverse”) transcytosis is assessed by measuring the delivery of a pIgR-targeting ligand into the circulation as measured by the presence of a detectable label that has been incorporated into the protein that is being tested. The integrity of the ligand recovered from the circulation can be assessed by analyzing the ligand on SDS polyacrylamide gel electrophoresis. Such assays are described in more detail in the Examples.

In vivo basolateral to apical (“forward”) transcytosis is assessed according to methods described in U.S. Pat. No. 6,072,041, which issued Jun. 6, 2000 to Davis et al.; U.S. Pat. No. 6,261,787 B1, which issued Jul. 17, 2001 to Davis et al.; published PCT application No. WO 00/53623, published Sep. 14, 2000, by Davis et al.; Eckman et al., Am J Respir Cell Mol Biol 1999 August;21(2):246-52; and Ferkol et al., Am. J. Respir. Crit. Care Med. 161:944-951, 2000.

I.C.6. Specificity of Binding

The binding of a ligand is target-specific in the sense that, although other molecules may be present in a mixture in which ligands and target molecules are contacted with each other, the ligand does not appreciably bind to other (non-target) molecules. For example, in the case of pIgR, it is recognized that the strength of binding between pIgR and a pIgR ligand, i.e., the affinity of a pIgR ligand for pIgR, is a matter of degree. As used herein, “target-specific” means that the pIgR ligand has a stronger affinity for its target molecule (pIgR) than for contaminating molecules, and this difference in affinity is sufficient for a given aspect of the invention. In general, the target specificity of a pIgR ligand for pIgR is comparable to the specificity of antibodies for their antigens. Thus, by way of non-limiting example, the specificity for a ligand for pIgR should be at least approximately that of a single chain antibody (sFv) for pIgR. Examples of sFv's that can be used to evaluate the target specificity of a pIgR ligand include but are not limited to sFv5A and derivatives thereof, such as sFv5AF, which bind to the stalk of pIgR and are described herein; and sFv's that bind to the secretory component (SC) such as, e.g., those described in U.S. Pat. No. 6,072,041.

The specificity of the binding is defined in terms of the values of absolute and relative binding parameters, such as the comparative dissociation constants (Kd) of a ligand for its target molecule as compared to the dissociation constant with respect to the ligand and unrelated molecules and compositions. Typically, the Kd of a ligand with respect to its target molecule will be 2-fold, preferably 5-fold, more preferably 10-fold less, than the Kd of the ligand for unrelated molecules and compositions. Even more preferably the Kd will be 50-fold less, more preferably 100-fold less, and more preferably 200-fold less.

The binding affinity of the ligands with respect to target molecules is defined in terms of the dissociation constant (Kd). The value of Kd can be determined directly by well-known methods, and can be computed even for complex mixtures by methods such as those, for example, set forth in Caceci, M., et al., Byte (1984) 9:340-362. In some situations, direct determination of Kd is problematic and can lead to misleadingly results. Under such circumstances, a competitive binding assay can be conducted to compare the affinity of a ligand for its target molecule with the affinity of molecules known to bind the target molecule. The value of the concentration at which 50% inhibition occurs (Ki) is, under ideal conditions, roughly equivalent to Kd. Moreover, Ki cannot be less than Kd; determination of Ki sets a maximal value for the value of Kd. Under circumstances where technical difficulties preclude accurate measurement of Kd, measurement of Ki can conveniently be substituted to provide, at the very least, an upper limit for Kd.

Kd may be measured in solution using techniques and compositions described in the following publications. Blake, D. A.; Blake, R. C.; Khosraviani, M.; Pavlov, A. R. “Immunoassays for Metal Ions.” Analytica Chimica Acta 1998, 376, 13-19. Blake, D. A.; Chakrabarti, P.; Khosraviani, M.; Hatcher, F. M.; Westhoff, C. M.; Goebel, P.; Wylie, D. E.; Blake, R. C. “Metal Binding Properties of a Monoclonal Antibody Directed toward Metal-Chelate Complexes.” Journal of Biological Chemistry 1996, 271(44), 27677-27685. Blake, D. A.; Khosraviani, M.; Pavlov, A. R.; Blake, R. C. “Characterization of a Metal-Specific Monoclonal Antibody.” Aga, D. S.; Thurman, E. M., Eds.; ACS Symposium Series 657; American Chemical Society: Washington, D.C., 1997; pp 49-60.

Binding constants and kinetic constants are estimated using calorimetry, equilibrium dialysis, and stopped flow methods using absorbance, fluorsescence, light scattering, turbidity, fluorescence anisotropy, and the like. Additionally or alternatively, Kd is measured using immobilized binding components on a chip, for example, on a BIAcore chip using surface plasmon resonance.

I.C.7. Surface Plasmon Resonance

Binding parameters are measured using surface plasmon resonance, for example, with a BIAcore® chip coated with immobilized binding components. Surface plasmon resonance is used to characterize the microscopic association and dissociation constants of reaction between an sFv or other ligand directed against pIgG associated molecules and pIgR and pIgR fragments. Such methods are generally described in the following references which are incorporated herein by reference. Vely F. et al., BIAcore analysis to test phosphopeptide-SH2 domain interactions, Methods in Molecular Biology. 121:313-21, 2000; Liparoto et al., Biosensor analysis of the interleukin-2 receptor complex, Journal of Molecular Recognition. 12:316-21, 1999; Lipschultz et al., Experimental design for analysis of complex kinetics using surface plasmon resonance, Methods. 20):310-8, 2000; Malmqvist., BIACORE: an affinity biosensor system for characterization of biomolecular interactions, Biochemical Society Transactions 27:335-40, 1999; Alfthan, Surface plasmon resonance biosensors as a tool in antibody engineering, Biosensors & Bioelectronics. 13:653-63, 1998; Fivash et al., BIAcore for macromolecular interaction, Current Opinion in Biotechnology. 9:97-101, 1998; Price et al.; Summary report on the ISOBM TD-4 Workshop: analysis of 56 monoclonal antibodies against the MUCI mucin. Tumour Biology 19 Suppl 1:1-20, 1998; Malmqvist et al, Biomolecular interaction analysis: affinity biosensor technologies for functional analysis of proteins, Current Opinion in Chemical Biology. 1:378-83, 1997; O'Shannessy et al., Interpretation of deviations from pseudo-first-order kinetic behavior in the characterization of ligand binding by biosensor technology, Analytical Biochemistry. 236:275-83, 1996; Malmborg et al., BIAcore as a tool in antibody engineering, Journal of Immunological Methods. 183:7-13, 1995; Van Regenmortel, Use of biosensors to characterize recombinant proteins, Developments in Biological Standardization. 83:143-51, 1994; and O'Shannessy, Determination of kinetic rate and equilibrium binding constants for macromolecular interactions: a critique of the surface plasmon resonance literature, Current Opinions in Biotechnology. 5:65-71, 1994.

BIAcore® uses the optical properties of surface plasmon resonance (SPR) to detect alterations in protein concentration bound within to a dextran matrix lying on the surface of a gold/glass sensor chip interface, a dextran biosensor matrix. In brief, proteins are covalently bound to the dextran matrix at a known concentration and a ligand for the protein (e.g., antibody) is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

II. Chemical Structures of Ligands and Targeting Elements

In complexes and compound of the invention, targeting elements and biologically active molecules are independently small molecules, nucleic acids or polypeptides.

Examples of compounds and moities that may be used as targeting elements in the compositions and compounds of the invention are as follows.

II.A. Small Molecules & Derivatives

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 2000 6(10):991-1007, Bioanalytical applications of accelerator mass spectrometry for pharmaceutical research; and Enjalbal et al., Mass Spectrom Rev 2000 19(3):139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

II.B. Nucleic Acids

Traditionally, techniques for detecting and purifying target molecules have used polypeptides, such as antibodies, that specifically bind such targets. Nucleic acids have long been known to specifically bind other nucleic acids (e.g., ones having complementary sequences). However, aptamers, nucleic acids that bind non-nucleic target molecules have been disclosed. See, e.g., Blackwell et al., Science (1990) 250:1104-1110; Blackwell et al., Science (1990) 250:1149-1152; Tuerk et al., Science (1990) 249:505-510; Joyce, Gene (1989) 82:83-87; and U.S. Pat. No. 5,840,867 entitled “Aptamer analogs specific for biomolecules”.

As applied to aptamers, the term “binding” specifically excludes the “Watson-Crick”-type binding interactions (i.e., A:T and G:C base-pairing) traditionally associated with the DNA double helix. The term “aptamer” thus refers to a nucleic acid or a nucleic acid derivative that specifically binds to a target molecule, wherein the target molecule is either (i) not a nucleic acid, or (ii) a nucleic acid or structural element thereof that is bound through mechanisms other than duplex- or triplex-type base pairing. Such a molecule is called a “non-nucleic molecule” herein.

II.B.1. Structures of Nucleic Acids

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

Nucleic acids that are aptamers are often, but need not be, prepared as oligonucleotides. Oligonucleotides include without limitation RNA, DNA and mixed RNA-DNA molecules having sequences of lengths that have minimum lengths of 2, 4, 6, 8, 10, 11, 12, 13, 14 or 15 nucleotides, and maximum lengths of about 100, 75, 50, 40, 25, 20 or 15 or more nucleotides, irrespectively. In general, a minimum of approximately 6 nucleotides, preferably 10, and more preferably 14 or 15 nucleotides, is necessary to effect specific binding.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is less susceptible to nuclease degradation than RNA. Similarly, chemical modifications that enhance an aptamer's specificity or stability are preferred.

II.B.2. Chemical Modifications of Nucleic Acids

Chemical modifications that may be incorporated into aptamers and other nucleic acids include with neither limitation nor exclusivity base modifications, sugar modifications, and backbone modifications.

II.B.2.a. Base Modifications

The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers.

II.B.2.b. Sugar Modifications

The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

II.B.2.c Backbone Modifications

Chemically modified backbones include, by way of non-limiting example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

II.B.3. Preparation and Identification of Aptamers

In general, techniques for identifying aptamers involve incubating a preselected non-nucleic target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′) is allowed to be randomly synthesized at that position (C-T-T-N-G-T, wherein N indicates a randomized position where, for example, the synthesizing reaction contains 25% each of A,T,C and G; or x % A, w % T, y % C and z % G, wherein x+w+y+z=100. In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting apatamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold (Science (1990) 249:505-510) disclose the use of a procedure termed “systematic evolution of ligands by exponential enrichment” (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterization. Kinzler, K. W., et al. (Nucleic Acids Res. (1989) 17:3645-3653) used a similar technique to identify synthetic double-stranded DNA molecules that are specifically bound by DNA-binding polypeptides. Ellington, A. D., et al. (Nature (1990) 346: 818-822) disclose the production of a large number of random sequence RNA molecules and the selection and identification of those that bind specifically to specific dyes such as Cibacron blue.

Another technique for identifying nucleic acids that bind non-nucleic target molecules is the oligonucleotide combinatorial technique disclosed by Ecker, D. J. et al. (Nuc. Acids Res. 21, 1853 (1993)) known as “synthetic unrandomization of randomized fragments” (SURF), which is based on repetitive synthesis and screening of increasingly simplified sets of oligonucleotide analogue libraries, pools and mixtures (Tuerk, C. and Gold, L. (Science 249, 505 (1990)). The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Nat'l. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

II.C. Polypeptides and Derivatives

As used herein, the term “polypeptide” includes proteins, fusion proteins, oligopeptides and polypeptide derivatives, with the exception that peptidomimetics are considered to be small molecules herein. Antibodies and antibody derivatives are disclosed in a separate section, but antibodies and antibody derivatives are, for purposes of the invention, treated as a subclass of the polypeptides and derivatives.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of protein that has an amino acid sequence that results from the linkage of the amino acid sequences of two or more normally separate polypeptides and which is encoded by a chimeric reading frame. Methods of preparing and using fusion proteins are disclosed in U.S. patent application Serial No. 60/237,929 (attorney docket No. 030854.0009 entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L.), filed Oct. 2, 2000, which is incorporated in its entirety herein.

A “protein fragment” is a proteolytic fragment of a larger polypeptide, which may be a protein or a fusion protein. A proteolytic fragment may be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” is a polypeptide having a short amino acid sequence (i.e., 2 to about 200 amino acids). An oligopeptide is generally prepared by chemical synthesis.

Although oligopeptides and protein fragments may be otherwise prepared, it is possible to use recombinant DNA technology and/or in vitro biochemical manipulations. For example, a nucleic acid encoding an amino acid sequence may be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabelled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique [Merrifield (1964), J. Am. Chem. Soc., 85: 2149; Stewart and Young (1984), Solid Phase polypeptide Synthesis, Pierce Chemical Company, Rockford, Ill.; Bodansky and Bodanszky (1984), The Practice of polypeptide Synthesis, Springer-Verlag, New York; Atherton and Sheppard (1989), Solid Phase polypeptide Synthesis: A Practical Approach, IRL Press, New York]. See, also, the specific method disclosed in Example 1 below.

Alternatively, polypeptides of this invention may be prepared in recombinant systems using polynucleotide sequences encoding the polypeptides. For example, fusion proteins are typically prepared using recombinant DNA technology.

II.C.1. Polypeptide Derivatives

A “derivative” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

II.C.1.a. Mutant Polypeptides

A polypeptide having an amino acid sequence identical to that found in a protein prepared from a natural source is a “wildtype” polypeptide. Mutant oligopeptides can be prepared by chemical synthesis, including without limitation combinatorial synthesis.

Mutant polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare mutant polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

II.C.1.b. Chemically Modified Polypeptides

As contemplated by this invention, the term “polypeptide” includes those having one or more chemical modification relative to another polypeptide, i.e., chemically modified polypeptides. The polypeptide from which a chemically modified polypeptide is derived may be a wildtype protein, a mutant protein or a mutant polypeptide, or polypeptide fragments thereof; an antibody or other polypeptide ligand according to the invention including without limitation single-chain antibodies, bacterial proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

II.C.1.b.1. Polypeptides with N-Terminal or C-Terminal Chemical Groups

An effective approach to confer resistance to peptidases acting on the N-terminal or C-terminal residues of a polypeptide is to add chemical groups at to one or both of the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al. (1993), Pharma. Res. 10: 1268-1273). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

II.C.1.b.2. Polypeptides with a Terminal D-Amino Acid

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

II.C.1.b.3. Polypeptides With Substitution of Natural Amino Acids By Unnatural Amino Acids

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. (1993), cited above).

II.C.1.b.4. Post-Translational Chemical Modifications

Different host cells will contain different post-translational modification mechanisms that may provide particular types of post-translational modification of a fusion protein if the amino acid sequences required for such modifications is present in the fusion protein. A large number (˜100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues. Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e, N-formyl-methionine (fMet). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274:22143-22146, 1999). In any event, in E. coli, the formyl group of fIMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the enzymes (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50:333-344, 1988, and Bradbury et al. Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

II.D. Peptidomimetics

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

II.D.1. Peptides With a Reduced Isostere Pseudopeptide Bond

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al. (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

II.D.2. Peptides With a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

II.D.3. Peptoid Derivatives

Peptoid derivatives of polypeptides represent another form of modified polypeptides that retain the important structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

III. Antibodies, Including Monoclonal Antibodies

The term “antibody” is meant to encompass an immunoglobulin molecule obtained by in vitro or in vivo generation of an immunogenic response, and includes both polyclonal, monospecific and monoclonal antibodies. An “immunogenic response” is one that results in the production of antibodies directed to one or more proteins after the appropriate cells have been contacted with such proteins, or polypeptide derivatives thereof, in a manner such that one or more portions of the protein function as epitopes. An epitope is a single antigenic determinant in a molecule. In proteins, particularly denatured proteins, an epitope is typically defined and represented by a contiguous amino acid sequence. However, in the case of nondenatured proteins, epitopes also include structures, such as active sites, that are formed by the three-dimensional folding of a protein in a manner such that amino acids from separate portions of the amino acid sequence of the protein are brought into close physical contact with each other.

Wildtype antibodies have four polypeptide chains, two identical heavy chains and two identical light chains. Both types of polypeptide chains have constant regions, which do not vary or vary minimally among antibodies of the same class (i.e, IgA, IgM, etc.), and variable regions. As is explained below, variable regions are unique to a particular antibody and comprise a recognition element for an epitope.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wildtype antibody are directed to the same epitope of an immunogenic protein, and a single wildtype antibody is thus capable of binding two molecules of the immunogenic protein at the same time.

III.A. Types of Antibodies

Compositions of antibodies have, depending on the manner in which they are prepared, different types of antibodies. Types of antibodies of particular interest include polyclonal, monospecific and monoclonal antibodies.

Polyclonal antibodies are generated in an immunogenic response to a protein having many epitopes. A composition of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

Monospecific antibodies (a.k.a. antipeptide antibodies) are generated in a humoral response to a short (typically, 5 to 20 amino acids) immunogenic polypeptide that corresponds to a few (preferably one) isolated epitopes of the protein from which it is derived. A plurality of monospecific antibodies includes a variety of different antibodies directed to a specific portion of the protein, i.e, to an amino acid sequence that contains at least one, preferably only one, epitope. Methods for producing monospecific antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A monoclonal antibody is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

Variants and derivatives of antibodies include antibody and T-cell receptor fragments that retain the ability to specifically bind to antigenic determinants. Preferred fragments include Fab fragments (i.e, an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (an Fab molecule having two antigen binding domains, each of which may be directed to a different epitope); a single chain Fab chain comprising a variable region, a.k.a., a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present.

The term “antibody” also includes genetically engineered antibodies and/or antibodies produced by recombinant DNA techniques and “humanized” antibodies. Humanized antibodies have been modified, by genetic manipulation and/or in vitro treatment to be more human, in terms of amino acid sequence, glycosylation pattern, etc., in order to reduce the antigenicity of the antibody or antibody fragment in an animal to which the antibody is intended to be administered (Gussow et al., Methods Enz. 203:99-121, 1991).

III.B. Methods of Preparing Antibodies and Antibody Variants

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems (the latter method of producing proteins is disclosed in more detail herein in the section entitled “Methods of Producing Fusion Proteins”). Antibodies and antibody variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (see, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Sacharomyces cerevisieae or Fichia pastoras harboring episomal or chromosomally integrated expression constructs; insect cells and viruses such as Sf 9 cells and baculovirus; and mammalian cells harboring episomal or chromosomally integrated (e.g., retroviral) expression constructs (for a review, see Verma et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

XenoMouse strains are genetically engineered mice in which the murine IgH and Igk loci have been functionally replaced by their Ig counterparts on yeast artificial YAC transgenes. These human Ig transgenes can carry the majority of the human variable repertoire and can undergo class switching from IgM to IgG isotypes. The immune system of the xenomouse recognizes administered human antigens as foreign and produces a strong humoral response. The use of XenoMouse in conjunction with well-established hybridomas techniques, results in fully human IgG mAbs with sub-nanomolar affinities for human antigens (see U.S. Pat. Nos. 5,770,429, entitled “Transgenic non-human animals capable of producing heterologous antibodies”; U.S. Pat. No. 6,162,963, entitled “Generation of Xenogenetic antibodies”; U.S. Pat. No. 6,150,584, entitled “Human antibodies derived from immunized xenomice”; U.S. Pat. No. 6,114,598, entitled Generation of xenogeneic antibodies; and U.S. Pat. No. 6,075,181, entitled “Human antibodies derived from immunized xenomice”; for reviews, see Green, Antibody engineering via genetic engineering of the mouse: XenoMouse strains are a vehicle for the facile generation of therapeutic human monoclonal antibodies, J. Immunol. Methods 231:11-23, 1999; Wells, Eek, a XenoMouse: Abgenix, Inc., Chem Biol 2000 August;7(8):R185-6; and Davis et al., Transgenic mice as a source of fully human antibodies for the treatment of cancer, Cancer Metastasis Rev 1999;18(4):421-5).

IV. Fusion Proteins

One type of compound of the invention is a fusion protein. A “fusion protein” is a single protein having amino acid sequences derived from two or more normally separate proteins, and which is encoded by a chimeric reading frame.

U.S. patent application Serial No. 60/237,929 (attorney docket Nos. 030854.0009 and 057220.0301) entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L., filed Oct. 2, 2000, is drawn to fusion proteins comprising pIgR ligands and biologically active polypeptides.

IV.A. Structure of Fusion Proteins and Chimeric Reading Frames

Polypeptides, which are polymers of amino acids, are encoded by another class of molecules, known as nucleic acids, which are polymers of structural units known as nucleotides. In particular, proteins are encoded by nucleic acids known as DNA and RNA (deoxyribonucleic acid and ribonucleic acid, respectively).

The nucleotide sequence of a nucleic acid contains the “blueprints” for a protein. Nucleic acids are polymers of nucleotides, four types of which are present in a given nucleic acid. The nucleotides in DNA are adenine, cytosine and guanine and thymine, (represented by A, C, G, and T respectively); in RNA, thymine (T) is replaced by uracil (U). The structures of nucleic acids are represented by the sequence of its nucleotides arranged in a 5′ (“5 prime”) to 3′ (“3 prime”) direction, e.g.,

5′-A-T-G-C-C-T-A-A-A-G-C-C-G-C-T-C-C-C-T-C-A-3′

In biological systems, proteins are typically produced in the following manner. A DNA molecule that has a nucleotide sequence that encodes the amino acid sequence of a protein is used as a template to guide the production of a messenger RNA (mRNA) that also encodes the protein; this process is known as transcription. In a subsequent process called translation, the mRNA is “read” and directs the synthesis of a protein having a particular amino acid sequence.

Each amino acid in a protein is encoded by a series of three contiguous nucleotides, each of which is known as a codon. In the “genetic code,” some amino acids are encoded by several codons, each codon having a different sequence; whereas other amino acids are encoded by only one codon sequence. An entire protein (i.e., a complete amino acid sequence) is encoded by a nucleic acid sequence called a reading frame. A reading frame is a continuous nucleotide sequence that encodes the amino acid sequence of a protein; the boundaries of a reading frame are defined by its initiation (start) and termination (stop) codons.

The process by which a protein is produced from a nucleic acid can be diagrammed as follows:

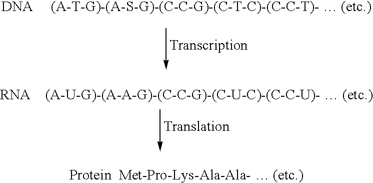

A chimeric reading frame encoding a fusion protein is prepared as follows. A “chimeric reading frame” is a genetically engineered reading frame that results from the fusion of two or more normally distinct reading frames, or fragments thereof, each of which normally encodes a separate polypeptide. Using recombinant DNA techniques, a first reading frame that encodes a first amino acid sequence is linked to a second reading frame that encodes a second amino acid sequence in order to generate a chimeric reading frame. Chimeric reading frames may also include nucleotide sequences that encode optional fusion protein elements (see below). A hypothetical example of a chimeric reading frame created from two normally separate reading frames is depicted in the following flowchart.

A first Reading Frame and “Protein-1”:

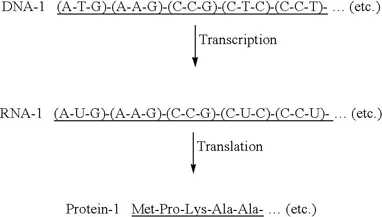

A second Reading Frame and “Protein-2”:

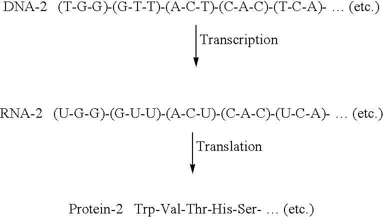

Chimeric Reading Frame that encodes a Fusion Protein that has sequences from Protein-1 and Protein-2:

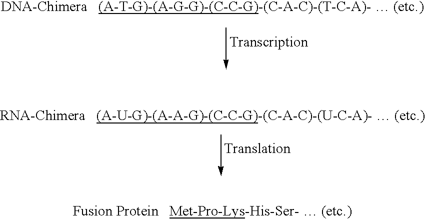

In order for a chimeric reading frame to be functional, each normally distinct reading frame therein must be fused to all of the other normally distinct reading frames in a manner such that all of the reading frames are in frame with each other. By “in frame with each other” it is meant that, in a chimeric reading frame, a first nucleic acid having a first reading frame is covalently linked to a second nucleic acid having a second reading frame in such a manner that the two reading frames are “read” (translated) in register with each other. As a result, the chimeric reading frame encodes one extended amino acid sequence that includes the amino acid sequences encoded by each of the normally separate reading frames.

A fusion protein of the invention comprises a polypeptide having the amino acid sequence of a monoclonal antibody and a polypeptide that is a targeting element. The targeting element may be an antibody derivative, such as a single-chain antibody, or some other polypeptide capable of binding the molecular target. Non-limiting examples of polypeptides that are pIgR-targeting elements are described in Example 1.

Methods of preparing fusion proteins are known in the art. White et al. (Protein Expr Purif 21: 446-455, 2001) describe cloning vectors that allow for the creation of fusion proteins having the framework (part of the constant region) of an IgG molecule linked to an amino-terminal domain that is introduced thereinto via genetic manipulation. One method of generating the fusion proteins of the invention is to use PCR and other cloning techniques to introduce the variable regions of a monoclonal antibody into such vectors, and adding to the amino terminus an amino acid sequence of a polypeptide that is a targeting element.

IV.B. Optional Fusion Protein Elements

In addition to pIgR targeting elements and biologically active polypeptides, the fusion proteins of the invention may further comprise one or more non-biologically active amino acid sequences, i.e., optional fusion protein elements. Such non biologically active elements include, but are not limited to, the following optional fusion protein elements. It is understood that a chimeric reading frame will include nucleotide sequences that encode such optional elements, and that these nucleotide sequences will be positioned so as to be in frame with the reading frame encoding the fusion protein. Optional fusion protein elements may be inserted between the pIgR-targeting element and the biologically active polypeptide, upstream or downstream (amino proximal and carboxy proximal, respectively) of these and other elements, or within the pIgR-targeting element and the biologically active polypeptide. A person skilled in the art will be able to determine which optional element(s) should be included in a fusion protein of the invention, and in what order, based on the desired method of production or intended use of the fusion protein.

Protein delivery elements are optional fusion protein elements that facilitate the uptake of a protein into cells but which are not pIgR targeting elements. The ETA (detoxified exotoxin a) protein delivery element is described in U.S. Pat. No. 6,086,900 to Draper. The VP22 protein delivery element is derived from herpes simplex virus-1 and vectors containing sequences encoding the VP22 protein delivery element are commercially available from Invitrogen (Carlsbad, Calif.; see also U.S. Pat. No. 6,017,735 to Ohare et al.). The Tat protein delivery element is derived from the amino acid sequence of the Tat protein of human immunodeficiency virus (HIV). See U.S. Pat. Nos. 5,804,604; 5,747,641; and 5,674,980.

Organellar delivery elements are optional fusion protein elements that direct a fusion protein into or out of a specific organelle or organelles. For example, the ricin A chain can be included in a fusion protein to mediate its delivery from the endosome into the cytosol. Additionally or alternatively, delivery elements for other organelles or subcellular spaces such as the nucleus, nucleolus, mitochondria, the Golgi apparatus, the endoplasmic reticulum (ER), the cytoplasm, etc. Mammalian expression constructs that incorporate organellar delivery elements are commercially available from Invitrogen (Carlsbad, Calif.: pShooter™ vectors). An H/KDEL (i.e, His/Lys-Asp-Glu-Leu sequence) may be incorporated into a fusion protein of the invention, preferably at the carboxy-terminus, in order to direct a fusion protein to the ER (see Andres et al., J. Biol. Chem. 266:14277-142782, 1991; and Pelham, Trends Bio. Sci. 15:483-486, 1990).

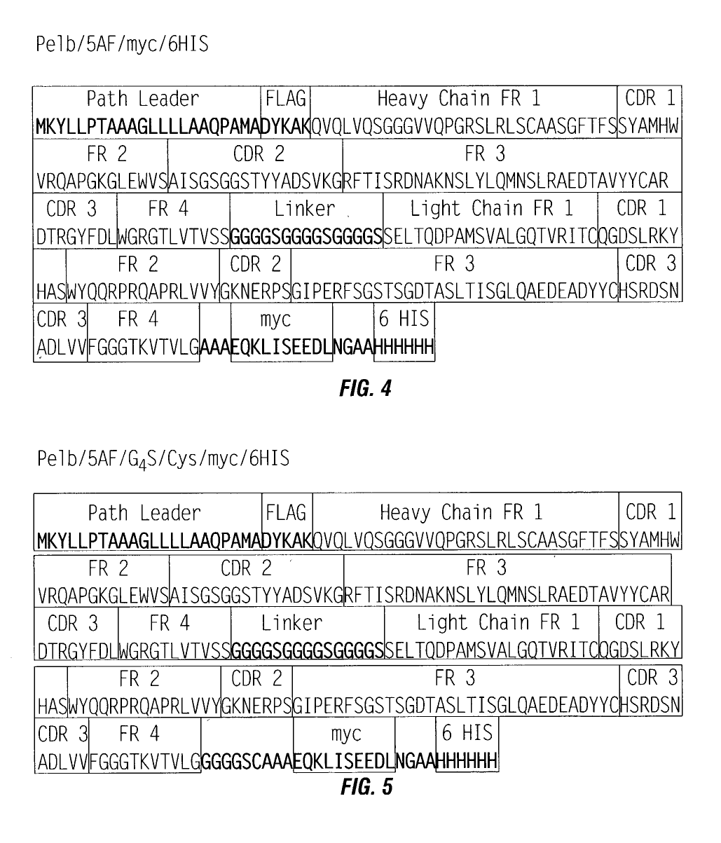

Another type of organellar delivery element is one which directs the fusion protein to the cell membrane and which may include a membrane anchoring element. Depending on the nature of the anchoring element, it can be cleaved on the internal or external leaflet of the membrane, thereby delivering the fusion protein to the intracellular or extracellular compartment, respectively. For example, it has been demonstrated that mammalian proteins can be linked to i) myristic acid by an amide-linkage to an N-terminal glycine residue, to ii) a fatty acid or diacylglycerol through an amide- or thioether-linkage of an N-terminal cysteine, respectively, or covalently to iii) a phophotidylinositol (PI) molecule through a C-terminal amino acid of a protein (for review, see Low, Biochem. J. 244:1-13, 1987). In the latter case, the PI molecule is linked to the C-terminus of the protein through an intervening glycan structure, and the PI then embeds itself into the phopholipid bilayer; hence the term “GPI” anchor. Specific examples of proteins know to have GPI anchors and their C-terminal amino acid sequences have been reported (see Table 1 and FIG. 4 in Low, Biochemica et Biophysica Acta, 988:427-454, 1989; and Table 3 in Ferguson, Ann. Rev. Biochem., 57:285-320, 1988). Incorporation of GPI anchors and other membrane-targeting elements into the amino- or carboxy-terminus of a fusion protein can direct the chimeric molecule to the cell surface.

Detectable polypeptides are optional fusion protein elements that either generate a detectable signal or are specifically recognized by a detectably labeled agent. An example of the former class of detectable polypeptide is green fluorescent protein (GFP). Examples of the latter class include epitopes such the “FLAG tag” and the c-myc epitope. These and other epitopes can be detected using labeled antibodies that are specific for the epitope; several such antibodies are commercially available.

Protein purification elements (a.k.a. protein isolation elements) are amino acid sequences that can be incorporated into a fusion protein in order to facilitate the purification or isolation of a fusion protein from a mixture containing other molecules.

Protein purification elements also include secretion sequences that direct recombinantly produced proteins out of the host cell and into the cellular media. Secreted proteins can then be separated from the host cells that produce them by simply collecting the media. Examples of secretion elements include those described in U.S. Pat. Nos. 5,846,818; 5,212,070; 5,631,144; 5,629,172; and 6,103,495; and Hardig et al., J. Biol. Chem. 268:3033-3036, 1993; Sizmann et al., Year Immunol. 7:119-130, 1993; and Power et al., Gene 113:95-99, 1992). Protein purification elements also include sequences that direct a recombinant protein to the periplasmic space of bacteria (Battistoni et al., Protein Expr. Purif. 6:579-587, 1995). Those skilled in the art will be able to determine which purification elements are desired, appropriate or necessary for a given fusion protein and/or expression system.

Of particular interest are purification elements that can be used to isolate a fusion protein from the host cells or media of an expression system. Examples of purification elements include a “His tag” (6 contiguous His residues, a.k.a. 6×His), which binds to surfaces that have been coated with nickel; streptavidin or avidin, which bind to surfaces that have been coated with biotin or “biotinylated” (see U.S. Pat. No. 4,839,293 and Airenne et al., Protein Expr. Purif. 17:139-145, 1999); and glutathione-s-transferase(GST), which binds glutathione (Kaplan et al., Protein Sci. 6:399-406, 1997; U.S. Pat. No. 5,654,176). Polypeptides that bind to lead ions have also been described (U.S. Pat. No. 6,111,079). “Epitope tags” such as the c-myc epitope or FLAG-tag can be used to purify recombinant proteins via affinity chromatography using antibodies to such epitope tags.

As used herein, the term “protein purification element” also includes elements designed to enhance the solubility and or assist in the proper folding of a protein. Such elements include GST and members of the 14-3-3 family of proteins (U.S. Pat. No. 6,077,689).

IV.C. Spacers

Spacers (a.k.a. linkers) are amino acid sequences that can be included in a fusion protein in between other portions of a fusion protein (e.g., between the biologically active polypeptide and the pIgR-targeting element, or between an optional fusion protein element and the remainder of the fusion protein). Spacers can be included for a variety of reasons. For example, a spacer can provide some physical separation between two parts of a protein that might otherwise interfere with each other via, e.g., steric hinderance. One example of a spacer of this type is the repeating amino acid sequence (Gly4-Ser)x, wherein x is 1 to 10, and preferably 1 to 4.

IV.D. Protease Cleavage Sites

In related embodiments of the invention, the pIgR-targeted fusion proteins can be designed so as to contain a site (a “protease cleavage site” or simply “cleavage site”) that is amenable to being cleaved by agents or under conditions that cause or promote such cleavage. In some preferred embodiments of the invention, the cleavage site is contained within a spacer element, so that cleavage separates, e.g., the pIgR targeting element of a fusion protein from the biologically active polypeptide thereof, which is useful for in vivo therapeutic methods; or between an optional protein purification element and the remainder of the fusion protein, which is useful for removing extraneous and potentially interfering purification elements in the process of purifying the fusion protein in vitro.

The nature and arrangement of a cleavage site or of a spacer containing a cleavage site will depend on the nature of the in vivo or in vitro method(s) of interest. It is understood by those skilled in the art that the amino acids sequences of fusion proteins that one wishes to have cleaved by a protease must be designed so as to retain the protease cleavage site of choice. Non-limiting examples of in vitro and in vivo cleavage sites and systems are as follows.

IV.D.1. In vivo Cleavage

Polypeptide fragments derived from the spacer and other optional fusion protein elements may be independently released from the cleaved fusion protein, or may remain associated with the pIgR targeting element or biologically active polypeptide. Most preferably, the cleavage reaction will predominantly occur after the fusion protein has been transported into or across an epithelial cell, or within a subcellular compartment, e.g., an organelle. For example, and for illustrative purposes only, the cleavage reaction might be effectuated by a protease or esterase found in an epithelial cell, by the acidic conditions found near a tumor cell, by conditions in the blood that destabilize disulfide conjugation, or by a protease found in an organelle.

Preferred cleavage sites for in vivo applications include but are not limited to those that are recognized by caspases, which can be used, e.g., to cleave and activate a biologically active polypeptide from a fusion protein during early events in apoptosis; proteases specific for an organelle into which it is desired to deliver a fusion protein, with one intended result being that a biologically active portion of the cleaved fusion protein will be retained by the organelle (i.e, organellar leader sequences).

Caspases are intracellular cysteine proteases which have been shown to play an essential role in the initiation and execution phases of apoptotic cell death. For reviews, see Fadeel et al. (IUBMB Life 49:421-425, 2000), Anderson (Cell Death Differ. 7:589-602, 2000) and Eamshaw et al., Annu. Rev. Biochem. 68:383-424, 1999). Fusion proteins can be designed so as to require proteolytic activation before it becomes biologically active. Inclusion of a given caspase cleavage site in such a fusion protein can be used to design fusion proteins that are cleaved by a particular caspase is activated. In instances where the biologically active component of a fusion protein is not active until released from the fusion protein, the latter type of fusion proteins provide biologically polypeptides that act at specific times during the apoptotic process. Cathepsins may be used in the same way in other vesicular compartnents of the cell.

Organellar leader sequences include, by way of non-limiting example, mitochondrial leader peptides that are proteolytically removed from proteins after their transport into mitochondria.

IV.D.2. In vitro Cleavage

Cleavable spacers may also be used for other purposes, especially in protein purification schemes. Consider, as an example, the case of a fusion protein that has an amino terminal 6×His tag, and a protease cleavage site located immediately carboxy terminal from the His tag, i.e, between the His tag and the remainder of the fusion protein being produced. After the fusion protein has been purified using the His tag's affinity for Nickel-coated surfaces, it is then cleaved with the appropriate protease in order to separate the His tag from the remainder of the protein. It is often desirable to remove elements such as His tags that are useful for protein purification purposes but might interfere with the biological activity of the fusion proteins. Cleavable spacers may be designed so as to regenerate the amino terminal amino acid sequence present in the original protein.

Preferred cleavage sites for in vitro applications include but are not limited to those that recognize a cleavage site, which may be introduced into a fusion protein by genetic manipulation, that is located between a portion of the fusion protein that is not required for, and may even be detrimental to, the in vivo uses for which the fusion protein is intended. Commercially available expression systems that may be used to introduce cleavage sites include by way of non-limiting example cleavage sites that are recognized by enterokinase, trypsin, Factor Xa, Factor IXa and thrombin.

Enterokinase may be used to cleave spacer elements (see U.S. Pat. No. 4,745,069). A preferred enterokinase is one that is produced via recombinant DNA techniques, as it is virtually free of other proteases and is able to efficiently cleave fusion proteins in partially purified preparations (Collins-Racie et al., Biotechnology 13:982-987, 1995). Moreover, enterokinase is relatively permissive regarding the amino acid residue downstream of the recognition sequence (Hosfield et al., Anal. Bochem. 269:10-16, 1999). Trypsin may also be used in this fashion (U.S. Pat. No. 6,037,143). In addition to providing cleavage sites for purification protein purposes, in vivo cleavage by gastrointestinal proteases such as enterokinase or trypsin may serve as a mechanism by which a fusion protein is released from a carrier in the gut.

Factor Xa (Peter et al., Circulation 101:1158-1164, 2000; U.S. Pat. No. 6,010,883) and thrombin are blood coagulation factors. Expression vectors may comprise a sequence encoding a cleavage site for thrombin or Factor Xa that can be used to remove a purification element (such as a His tag) from the fusion protein after it has served its purification purpose.

IV.E. Production of Fusion Proteins via Recombinant DNA Expression Systems

In order to achieve recombinant expression of a fusion protein, an expression cassette or construct capable of expressing a chimeric reading frame is introduced into an appropriate host cell to generate an expression system. The expression cassettes and constructs of the invention may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

Host cells which may be used in the expression systems of the present invention are not strictly limited, provided that they are suitable for use in the expression of the chimeric pIgR-targeting peptide of interest. Suitable hosts may often include eukaryotic cells. Preferred eukaryotic hosts include, for example, yeast, fungi, insect cells, mammalian cells either in vivo, or in tissue culture.

Expression cassettes and constructs may be introduced into an appropriate host cell by any of a variety of suitable means, i.e, transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a chimeric pIgR-targeting peptide of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

A variety of recombinant DNA expression systems may be used to produce the fusion proteins of the invention. Expression systems of particular interest include prokaryotic systems, yeast expression systems, insect expression systems mammalian expression systems.

Prokaryotic Expression Systems utilize plasmid and viral (bacteriophage) expression vectors that contain replication sites and control sequences derived from a species compatible with the host may be used. Suitable phage or bacteriophage vectors may include λgt10, λgt11 and the like; and suitable virus vectors may include pMAM-neo, pKRC and the like. Appropriate prokaryotic plasmid vectors include those capable of replication in E. coli (such as, by way of non-limiting example, pBR322, pUC118, pUC119, ColE1, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as ΦC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978). See also Brent et al., “Vectors Derived From Plasmids,” Section II, and Lech et al. “Vectors derived from Lambda and Related Bacteriophages” Section III, in Chapter 1 of Short Protocols in Molecular Biology, 2nd Ed., Ausubel et al., eds., John Wiley and Sons, New York, 1992, pages 1-13 to 1-27; Lech et al. “Vectors derived from Lambda and Related Bacteriophages” Section III and Id. pages 1-28 to page 1-52.

Recognized prokaryotic hosts include bacteria such as E. coli, Bacillus, Streptomyces, Pseudomonas, Salmonella, Serratia, and the like. However, in such hosts, the fusion protein will not be glycosylated. In any event, the host cell must be compatible with the replicon and control sequences in the expression cassette.

To express a chimeric pIgR-targeting peptide of the invention (or a functional derivative thereof) in a prokaryotic cell, it is necessary to operably link the sequence encoding the chimeric pIgR-targeting peptide of the invention to a functional prokaryotic promoter. Such promoters may be either constitutive or, more preferably, regulatable (i.e, inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (PL and PR), the trp, recA, lacZ, lac, and gal promoters of E. coli, the α-amylase (Ulmanen et al., J. Bacteriol. 162:176-182, 1985) and promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Bacterial systems may also be used to create and produce large amounts of shuttle vectors. Shuttle vectors are constructs designed to replicate in a prokaryotic host such as E. coli but which contain sequences that allow the shuttle vector and a chimeric reading frame incorporated therein to be transferred to a eukaryotic viral vector or other vector such as baculovirus or adenovirus.

Yeast Expression Systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast cells provide a substantial advantage over prokarytoic expression systems in that they can carry out post-translational modifications of fusion proteins. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e, pre-peptides).

Preferred yeast expression vectors include those derived from the episomal element known as the 2-micron circle as well as derivatives of yeast integrating (YIp), yeast replicating (YRp), yeast centromeric (YCp), yeast episomal (YEp), and yeast linear (YLp) plasmids (Broach, in: The Molecular Biology of the Yeast Saccharomyces: Life Cycle and Inheritance, Cold Spring Harbor Laboratory, Cold Spring Harbor, N.Y., p. 445-470, 1981; Lundblad et al., Section II and, Becker et al., Section III, of Chapter 13 in: Short Protocols in Molecular Biology, 2nd Ed., Ausubel et al., eds., John Wiley and Sons, New York, 1992, pages 13-19 to 13-41).

Insect Expression Systems utilize insect host cells, e.g., sf9 and sf21 cells, both of which are derived from the iplbsf-21 cell line derive from the pupal ovarian tissue of the fall army worm spodoptera frugiperda (O'Reilly et al., Baculovirus expression vectors: A Laboratory Manual New York, N.Y., W. H. Freeman and Company. See also baculovirus expression protocols in Methods in Molecular Biology Vol. 39; Richardson ed. Humana Totowa N.J., 1992; and Vaughn et al., In vitro 13:213-217, 1977. The cell line bti-tn-5b1-4 (high 5 tm cell line), which originated from the ovarian cells of the cabbage luper, Trichoplusa ni (Davis et al., Biotechnology 10:1148-1150, 1992; Granados et al., J. Invertebr. Pathol. 64:260-266, 1994; Wickham et al., Biotechnology Prog. 8:391-396, 1992; Wickham et al., Biotechnol. Prog. 9:25-30, 1993). Other insect cell lines that can be used to express baculovirus vectors have been described (Hink et al., Biotechnol. Prog. 7:9-14, 1991). See, also Piwnica-Worms “Expression of Proteins in Insect Cells Using Baculo Viral Vectors” section II in chapter 16 of Short Protocols in Molecular Biology, second edition, Ausubel et al, eds., John Wiley and Sons, New York, N.Y. 1992. Using insect cells as hosts, the Drosophila alcohol dehydrogenase promoter can be used (Rubin, Science 240:1453-1459, 1988). Alternatively, baculovirus vectors can be engineered to express large amounts of chimeric pIgR-targeting peptides of the invention in insect cells (Jasny, Science 238:1653, 1987; Miller et al., in: Genetic Engineering, Vol. 8, Plenum, Setlow et al., eds., pp. 277-297, 1986).

Mammalian Expression Systems utilize host cells such as HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

Several expression vectors are available for the expression of chimeric pIgR-targeting peptides of the invention in a mammalian host. A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, in: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Expression of chimeric pIgR-targeting peptides of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).]

Translation of eukaryotic mRNA is initiated at the codon which encodes the first methionine. For this reason, it is preferable to ensure that the linkage between a eukaryotic promoter and a DNA sequence which encodes a chimeric pIgR-targeting peptide of the invention (or a functional derivative thereof) does not contain any intervening codons which are capable of encoding a methionine (i.e, AUG). The presence of such codons results either in the formation of a fusion protein (if the AUG codon is in the same reading frame as the chimeric pIgR-targeting peptide of the invention coding sequence) or a frame-shift mutation (if the AUG codon is not in the same reading frame as the chimeric pIgR-targeting peptide of the invention coding sequence).

V. Protein Conjugates

One type of compound of the invention is a protein conjugate, i.e., a Mab that is covalently linked to a targeting element that is a polypeptide.

V.A. Covalently Attaching Targeting Elements to Bioactive Compounds

Polypeptides that are pIgR-targeting elements, including but not limited to antibody derivatives and bacterial proteins that bind pIgR, can be linked to bioactive compounds in a varity of ways. In general, there are four ways that protein conjugate members are linked to other protein conjugate members. First, amino acid residues present in the natural sequence of a first protein member can be directly covalently linked to amino acid residues in the natural amino acid sequence of a second protein member as in, e.g., a disulfide bridge. Second, mutant amino acids useful for covalent linkages can be introduced into one or more protein members by using molecular genetics to alter the reading frame encoding such protein members or, in the case of synthetic oliogopeptides, directly during the in vitro synthesis thereof. Third, natural or mutant amino acid sequences present in isolated proteins can be “derivatized” (i.e, chemically modified in vitro) so as to include chemical groups not present in natural amino acids but useful for the chemical conjugation of oligopeptides, polypeptides, and proteins in a related methodology, unnatural amino acids having moities useful for chemical conjugation are introduced into oligopeptides or peptidomimetics during their synthesis in vitro. Fourth, a cross-linking reagent (a.k.a. “cross-linker”), typically a bifunctional (two-armed) chemical linker that forms covalent linkages between two or more conjugate members, can be used to covalently link conjugate members to each other. Such bifunctional linkers can be homobifunctional (wherein both “arms” of the linker are the same chemical moiety) or heterobifunctional (wherein each of the two “arms” is a different chemical moiety than the other).

Hermanson (Bioconjugate Techniques, Academic Press, 1996), herein incorporated by reference, summarizes many of the chemical methods used to link proteins and other molecules together using various reactive functional groups present on various cross-linking or derivatizing reagents. Cross-linking agents are based on reactive functional groups that modify and couple to amino acid side chains of proteins and peptides, as well as to other macromolecules. Bifunctional cross-linking reagents incorporate two or more functional reactive groups. The functional reactive groups in a bifunctional cross-linking reagent may be the same (homobifunctional) or different (heterobifunctional). Many different cross-linkers are available to cross-link various proteins, peptides, and macromolecules. Table 7 lists some of the cross-linkers that are available through commercial sources according to their class of chemical reactivity. Table 8 lists some of the properties of chemical cross-linkers and the types of functional groups with which they react.

TABLE 7
CLASSES OF CHEMICAL REACTIVITY OF CROSS-
LINKERS AND EXAMPLES OF CROSSLINKERS THAT
CARRY OUT CROSS-LINKING FUNCTIONS

| Chemical reactivity | Abbreviation | Compound |
|---|---|---|
| Homobifunctional imidoesters | DMA | Dimethyl adipimidate.2 HCl |
| | DMP | Dimethyl pimelimidate.2 HCl |
| | DMS | Dimethyl suberimidate.2 HCl |
| | DTBP | Dimethyl 3,3′-dithiobispropionimidate. |
| | | 2HCl |
| Homobifunctional N- | DSG | Disuccinimidyl glutarate |
| hydroxysuccinimide esters (NHS- | | |
| esters) | | |
| | DMSC | Dimethyl succinimidate.2 HCl |
| | DSS | Disuccinimidyl suberate |
| | BS | |
| | DSP | Dithiobis(succinimidylpropionate) |
| | DTSSP | Dithiobis(sulfosuccinimidylpropionate) |
| | DTME | Dithio-bis-maleimidoethane |
| | EGS | Ethylene glycolbis(succinimidylsuccinate) |
| | Sulfo-EGS | Ethylene |
| | | glycolbis(sulfosuccinimidylsuccinate) |
| | DST | Disuccinimidyl tartrate |
| | Sulfo-DST | Disulfosuccinimidyl tartrate |
| | BSOCOES | Bis[2- |
| | | (succinimidooxycarbonyloxy)ethyl]sulfone |
| | Sulfo-BSCOCOES | Bis[2- |
| | | (sulfosuccinimidooxycarbonyloxy)ethyl]sulfone |
| heterobifunctional NHS-esters | BS3 | BIS-(sulfosuccinimidyl) suberate |
| | DMM | dimethyl malonimidate • 2 HCl |
| | EMCS | N-[ε-maleimidocaproyloxy]succinimide |
| | | ester |
| | Sulfo-EMCS | N-[ε- |
| | | maleimidocaproyloxy]sulfosuccinimide |
| | | ester |
| | SMCC | Succinimidyl 4-(N- |
| | | maleimidomethyl)cyclohexane-1- |
| | | carboxylate |
| | LC-SMCC | Succiminidyl-4-(N- |
| | | maleimidomethyl)cyclohexane-1-carboxy- |
| | | (6-amido-caproate) |
| | Sulfo-MBS | m-maleimidobenzoyl-N- |
| | | hydoxysulfosuccinimide ester |
| | Sulfo-SMCC | Sulfosuccinimidyl 4-(N- |
| | | maleimidomethyl)cyclohexane-1- |
| | | carboxylate |
| | MBS | m-maleimidobenzoyl-N- |
| | | hydoxysuccinimide ester |
| | SMPB | Succinimidyl 4-[P-Maleimidophenyl] |
| | | butyrate |
| | Sulfo-SMPB | Sulfosuccinimidyl 4-[p- |
| | | maleimidophenyl]butyrate |
| | BMH | Bismaleimidohexane |
| | GMBS | N-[γ-Maleimidobutyryloxy] succinimide |
| | | ester |
| | Sulfo-GMBS | N-[γ-Maleimidobutyryloxy] |
| | | sulfosuccinimide ester |
| heterobifunctional haloacetyl | SIAB | N-succinimidyl(4- |
| NHS-esters | | iodoacetyl)aminobenzoate |
| | Sulfo-SIAB | Sulfo-SIAB sulfosuccinimidyl(4- |
| | | iodoacetyl)aminobenzoate |
| homobifunctional pyridyldithiols | DPDPB | 1,4-Di-[3′-(2′- |
| | | pyridyldithio)propionamido]butane |
| heterobifunctional pyridyldithiols | SMPT | 4-succinimidyloxycarbonyl-methyl-(2- |
| | | pyridyldithio)-toluene |
| | Sulfo-LC- | Sulfosuccinimidyl 6-[a-methyl-a-(2- |
| | SMPT | pyridyl-dithio)toluamido]hexanoate |
| | SPDP | N-succinimidyl 3-(2- |
| | | pyridyldithio)propionate |
| | LC-SPDP | N-succinimidyl 6-[3’-(2- |
| | | pyridyldithio)propionamido]hexanoate |
| | Sulfo-LC- | Sulfosuccinimidyl 6-[3’-(2-pyridyldithio)- |
| | SPDP | propionamido] hexanoate |
| carboxyl reactive | PDPH | 3-(2-Pyridyldithio) propionyl hydrazide |
| carbonyl reactive | | |
| | EDC | 1-ethyl-3-(3-dimethylaminopropyl)- |
| | | carbodiimide |
| | M2C2H | 4-(N-Maleimidomethyl)cyclohexane-1- |
| | | carboxyl hydrazide |
| | DCC | N,N-dicyclohexylcarbodimide |
| | MPBH | 4-(4-N-Maleimidophenyl)butyric acid |
| | | hydrazide hydrochloride |
| Photoreactive | ABH | Azidobenzoyl hydrazide |
| | ANB-NOS | N-5-azido-2-nitrobenzoyloxysuccinimide |
| | APDP | N-[4-(p-azidosalicylamido)butyl]-3’(2’- |
| | | pyridyldithio)propionamide |
| | APG | p-Azidophenylglyoxal monhydrate |
| | ASBA | 4-(p-Azidosalicylamido)butylamine |
| | ASIB | 1-(p-Azidosalicylamido)-4- |
| | | (iodoaceamido)butane |
| | BASED | Bis-[B-4- |
| | | azidosalicylamido)ethyl]disulfide |
| | HSAB | N-Hydroxysuccinimidyl-4-azidobenzoate |
| | Sulfo-HSAB | N-Hydroxysulfo-succinimdyl-4-azidobenzoate |
| | NHS-ASA | N-Hydroxysuccinimidyl-4-azidosalicylic |
| | | acid |
| | Sulfo-NHS-ASA | N-Hydroxysulfo-succinimidly-4- |
| | | azidosalicylic acid |
| | Sulfo-NHS- | Sulfosuccinimidly-[4-azidosalicylamido)- |
| | LC-ASA | hexanoate |
| | PNP-DTP | p-Nitropheyno-2-diazo-3,3,3- |
| | | trifluoropropionate |
| | DTP | 2-Diazo-3,3,3-trifluoropropionylchloride |
| | SADP | N-succinimidyl-(4-azidopheynyl 1,3’ |
| | | dithiopropionate |
| | Sulfo-SADP | Sulfosuccinimidyl-(4- |
| | | azidophynyldithio)propionate |
| | SAED | Sulfosuccinimidyl 2(7-azido-4- |
| | | methylcoumarin-3-acetamide)ethyl-1,3 |
| | | -dithiopropionate |
| | Sulfo-SAMCA | Sulfosuccinimidyl 7-azido-4- |
| | | methycoumarin-3-acetate |
| | SAND | Sulfosuccinimidyl 2-(m-azido-o- |
| | | nitrobenzamdio)-ethyl-1,3- |
| | | dithiopropionate |
| | SANPH | N-succinimidyl-6-(4’-azido-2’- |
| | | nitrophenylamino)hexanoate |
| | Sulfo-SANPH | Sulfosuccinimidyl 6-(4’-azido-2’- |
| | | nitrophenylamino)hexanoate |
| | SASD | Sulfosuccinimidyl 2-(p- |
| | | azdiosalicylamido)ethyl-1,3’- |
| | | dithiopropionate |
| | Sulfo-SAPB | Sulfosuccinimidyl 4-(p-azidophenyl)- |
| | | butyrate |
| Heterobifunctional amine reactive | SDBP | N-Hydroxysuccinimidyl 2,3- |
| | | dibromopropionate |
| Bifunctional aryl halide | DFDNB | 1,5-Difluoro-2.4-dinitrobenzene |
| heterobifunctional | mal-sac-HNSA | maleimido-6-aminocaproyl-ester of |
| nitrophenylsulfonic acid ester | | 1-hydroxy-2-nitrobenzene-4-sulfonic acid |

TABLE 8
CHEMICAL CROSS-LINKERS AND SOME OF THEIR PROPERTIES

| | Pierce | Spacer Arm | | | | |
|---|---|---|---|---|---|---|
| | Product | Length | | Cleavable | Water | Membrane |
| Acronym | Number | (angstroms) | Links | By | Soluble | Permeable |
| Sulfo-LC-SM | 21568 | 20.0 | Amines To | Thiols | Yes | No |
| PT | | | Sulfhydryls | | | |
| SMPT | 21558 | 20.0 | Amines To | Thiols | Yes | No |
| | | | Sulfhydryls | | | |
| Sulfo-KMUS | 21111 | 19.0 | Amines To | non | Yes | No |
| | | | Sulfhydryls | | | |
| LC-SMCC | 22362 | 16.1 | Amines To | non | Yes | No |
| | | | Sulfhydryls | | | |
| KMUA | 22211 | 15.7 | Amines To | non | Yes | No |
| | | | Sulfhydryls | | | |
| LC-SPDP | 21651 | 15.6 | Amines To | non | No | N/d |
| | | | Sulfhydryls | | | |
| Sulfo-LC-SP | 21650 | 15.6 | Amines To | Thiols | Yes | No |
| DP | | | Sulfhydryls | | | |
| SMPB | 22416 | 14.5 | Amines To | non | No | Yes |
| | | | Sulfhydryls | | | |
| Sulfo-SMPB | 22317 | 14.5 | Amines To | non | Yes | No |
| | | | Sulfhydryls | | | |
| SMPH | 22363 | 14.3 | Amines To | non | No | N/d |
| | | | Sulfhydryls | | | |
| SMCC | 22360 | 11.6 | Amines to | non | No | Yes |
| | | | Sulfhydryls | | | |
| Sulfo-SMCC | 22322 | 11.6 | Amines to | non | Yes | No |
| | | | Sulfhydryls | | | |
| SIAB | 22329 | 10.6 | Amines to | non | No | Yes |
| | | | Sulfhydryls | | | |
| Sulfo-SIAB | 22327 | 10.6 | Amines To | non | Yes | No |
| | | | Sulfhydryls | | | |
| Sulfo-GMBS | 22324 | 10.2 | Amines To | non | Yes | No |
| | | | Sulfhydryls | | | |
| GMBS | 22309 | 10.2 | Amines To | non | No | Yes |
| | | | Sulfhydryls | | | |
| MBS | 22311 | 9.9 | Amines To | non | No | Yes |
| | | | Sulfhydryls | | | |
| Sulfo-MBS | 22312 | 9.9 | Amines To | non | Yes | No |
| | | | Sulfhydryls | | | |
| Sulfo-EMCS | 22307 | 9.4 | Amines To | non | Yes | No |
| | | | Sulfhydryls | | | |
| EMCA | 22306 | 9.4 | Amines To | non | Yes | No |
| | | | Sulfhydryls | | | |
| EMCS | 22308 | 9.4 | Amines To | non | No | Yes |
| | | | Sulfhydryls | | | |
| SVSB | 22358 | 8.3 | Amines To | non | No | Yes |
| | | | Sulfhydryls | | | |
| BMPS | 22298 | 6.9 | Amines To | non | No | N/d |
| | | | Sulfhydryls | | | |
| SPDP | 21857 | 6.8 | Amines To | Thiols | No | Yes |
| | | | Sulfhydryls | | | |
| SBAP | 22339 | 6.2 | Amines To | non | No | Yes |
| | | | Sulfhydryls | | | |
| BMPA | 22296 | 5.9 | Amines To | non | Yes | No |
| | | | Sulfhydryls | | | |
| AMAS | 22295 | 4.4 | Amines To | non | No | N/d |
| | | | Sulfhydryls | | | |
| SATP | 26100 | 4.1 | Amines To | non | No | Yes |
| | | | Sulfhydryls | | | |
| SIA | 22349 | 1.5 | Amines To | non | No | N/d |
| | | | Sulfhydryls | | | |
| Sulfo-LC-SM | 21568 | 20.0 | Sulfhydryls | Thiols | Yes | No |
| PT | | | to Amines | | | |
| SMPT | 21558 | 20.0 | Sulfhydryls | Thiols | No | Yes |
| | | | to Amines | | | |
| AEDP | 22101 | 9.5 | Carboxyls to | Thiols | Yes | No |
| | | | Amines | | | |
| EDC | 22980 | 0.0 | Carboxyls to | non | Yes | No |
| | | | Amines | | | |

Bifunctional cross-linking reagents may be classified according to their functional groups, chemical specificity, length of the cross bridge that they establish, the presence of similar functional groups or dissimilar functional groups, chemical or photochemical reactivity, ability to be cleaved internally by reduction or other means, and the ability of the reagent to be further modified by radiolabelling (i.e. radioiodination) or addition of detectable tags or labels. The selective groups on the cross-linking reagent can be present in a homobifunctional arrangement in which the selective groups are identical, or can be present in a heterobifunctional arrangement in which the selective groups are dissimilar.

The chemical modification may be done using cross-linking reagents containing selective groups that react with primary amines, sulfhydryl (thiol) groups, carbonyl, carboxyl groups, hydroxyl, or carbohydrates and other groups placed on a protein or peptide, especially by posttranslational modifications within the cell. The selective groups include, but are not limited to, imidoester, N-hydroxysuccinimide ester or sulfosuccinimidyl ester, ester of 1-hydroxy-2-nitrobenzene-4-sulfonic, maleimide, pyridyl disulfide, carbodiimide, and a-haloacetyl groups.

Sulfhydryl reactive functional groups include maleimides, alkyl and aryl halides, α-haloacyls, and pyridyl disulfides. Maleimides, alkyl and aryl halides, and α-haloacyls react with thiols to form stable thioether bonds that are not reduced by reagents such as 2-mercaptoethanol and dithiothreitol. Pyridyl disulfides form mixed disulfides with thiol groups, mixed disulfides may be used as an intermediate for cross-linking two or more macromolecules. Cross-linkers that first react with a carboxyl group to form an activated intermediate and then reacts with an amino group, such as a ε-amino group of lysine or an α-amino group of an amino terminal amino acid, may be used.

A spacer arm or “cross-bridge” region, consisting of a spacer group or a functional group, such as a disulfide bond or hindered disulfide bond, may be used to connect the Mab to the targeting element. The length of the spacer arm may be varied. The distance between the functional groups establishes the length of the spacer arm. Longer spacer arms may be required to diminish or eliminate steric hindrance between two molecules that are cross-linked together. Intermolecular cross-linking is more efficient with longer spacer arms. Short spacer arms favor intramolecular cross-linking, which is preferably avoided in the present invention.

Spacer arms may have reactive bonds within them that enable further modifications. For example, internal cleavable bonds may be placed within the spacer, such as disulfides or hindered disulfides, one or more ester bonds, or vicinal hydroxyl groups. Cleavage of internal disulfide bonds may be achieved using reduction with thiol containing reagents such as 2-mercaptoethanol and dithiothreitol. One or more metabolizable bonds may be inserted internally in the cross-linking reagent to provide the ability for the coupled entities to separate after the bond(s) is broken after the conjugate is transported into the cell and into the body.

Homobifunctional cross-linkers contain at least two identical functional groups. Heterobifunctional cross-linkers contain two or more functional reactive groups that react with different specificity. Because heterobifunctional cross-linkers contain different reactive groups, each end can be individually directed towards different functional groups on proteins, peptides, and macromolecules. This feature results in linking, for example, amino groups on one molecular entity to carboxyl groups on another entity, or amino groups on one entity to sulfhydryl groups on another entity.

Functional groups include reactive portions on proteins, peptides, and macromolecules that are capable of undergoing chemical reaction. Functional groups include amino and carboxyl groups, hydroxyl groups, phenolate hydroxyl groups, carbonyl groups, guanidinyl groups, and carbon-carbon double bonds. In addition, photoactive reagents that become reactive when exposed to light may be used. For example, arylazides may be activated to form activated intermediates, such as an aryl nitrene or a dehydroazepine intermediate, that non-selectively inserts into carbon-hydrogen bonds (i.e. by aryl nitrenes) or reacts with amines (dehydroazepines). Other examples include fluorinated aryl azides, benzophenones, certain diazo compounds, and diazrine derivatives.

V.A.1. N-Hydroxysuccinimide Esters

NHS-esters react efficiently with amino groups in aqueous buffers, preferably phosphate, bicarbonate/carbonate, HEPES, and borate buffers at concentrations between 10 and 200 mM. Buffers should not contain primary amines. Primary amines can be added to the reaction to stop or quench the NHS-ester reaction and thereby terminate further modification of amino groups on proteins, peptides, and macromolecules. The modification or coupling is typically carried out between pH 7 and pH 9, and preferably between pH 7.5 and 8.0. The time of reaction and temperature may depend on the particular molecule that is being modified. The time of modification may be between 10 and 180 minutes, preferably between 30 and 60 minutes at temperatures between 4° C. and 37° C., preferably between 4° C. and 25° C. The concentration of the NHS-ester may vary, but is between 1.1- to 100-fold molar excess, and preferably between 1.1- and 10-fold molar excess. The protein concentration may vary between 1 μM and 100 μM, preferably between 5 μM and 100 μM.

V.A.2. Ester of 1-Hydroxy-2-Nitrobenzene-4-Sulfonic Acid

A maleimido-aliphatic carboxylic acid may form an ester with the hydroxyl group of 1-hydroxy-2-nitrobenzene-4-sulfonic acid. A maleimide group may be placed at the end of a short, intermediate, or long aliphatic acid. An example of this is mal-sac-HNSA (U.S. Pat. No. 4,954,637). Mal-sac-HNSA may be used to acylate amino groups on proteins, peptides, and macromolecules. The maleimide may then be reacted with sulfhydryl groups on other proteins, peptides, and macromolecules to form a stable, noncleavable thioether bond. Aqueous buffers, such as sodium phosphate, and neutral to mildly alkaline conditions, pH 6.5 to 9, and preferably pH 7 to 8, may be used at temperatures from 0° C. to 37° C., and preferably from 4° C. to 25° C.

V.B. Moieties That May be Modified on Macromolecules for Chemical Cross-Linking

V.B.1. Naturally Occurring Modifiable Moieties

Proteins and peptides contain α-amino groups at the amino terminus, ε-amino groups on lysine, β-carboxyl groups on aspartic acid, γ-carboxyl groups on glutamic acid, imidazole rings on histidine, hydroxyl groups on serine and threonine, phenolate hydroxyl groups on tyrosine, sulfhydryl groups on cysteine, disulfide bonds between two cysteines, mercaptide bonds in methionine, and indole rings in tryptophan, all of which can be selectively modified by cross-linking reagents.

Carbohydrates or carbohydrate containing macromolecules contain ketone, aldehyde, hydroxyl, amine, carboxylate, sulfate, and phosphate groups as nonlimiting examples of functional groups with which cross-linkers may react. Carbohydrates containing vicinal hydroxyl groups (hydroxyl groups on adjacent carbon atoms) may be treated with sodium periodate so that the carbon-carbon bond is cleaved; this creates reactive formyl groups on the treated carbohydrate that may be used as a target for appropriately designed cross-linking reagents. Hermanson discloses other methods, which are herein incorporated by reference, for cross-linking carbohydrates.

Hermanson discloses the major sites on nucleic acids that are susceptible to chemical modification. Compositions and methods for synthesizing and conjugating oligonucleotides comprising a Cys residue have been described by Stetsenko and Gait (Nucleosides, Nucleotides and Nucleic Acids 19:1751-1764, 2000).

V.B.2. Substitution or Insertion of Cysteine into Polypeptides for Subsequent Chemical Modification

Ligands genetically fused to therapeutic and diagnostic biologically active proteins and peptides may not always produce a desired result. Genetic fusion is typically performed by attaching the ligand to either the amino or carboxy terminus of the biologically active protein or peptide using a spacer if necessary. Therefore, the geometrical arrangement of ligand and biologically active protein and peptide is necessarily limited. Linkage of the biologically active protein or peptide through surface cysteinyl groups presents more flexibility in designing a combination that allows both the ligand to recognize pIgR and the biologically active protein or peptide to carry out its functions after epithelial transport. If the desired sulfhydryl groups are not present on the protein, peptide or macromolecule, a sulfhydryl may be introduced by genetic modification. Therefore, the present invention provides a method for substituting or inserting a cysteine into the protein or peptide and using the cysteine for chemical conjugation by cross-linking.

An amino acid may be selected for substitution by cysteine, such selected amino acid should be on the surface of the molecule and positioned so as not to interfere or sterically hinder the function of any biologically active site or important site on the molecule that is required for a biological activity or function. The substitution of cysteine for an amino acid may be achieved by methods well-known to those skilled in the art, for example, by using methods described in Maniatis, Sambrook, and Fritsch (Molecular Cloning: A laboratory manual, Cold Spring Harbor Laboratory Press, 1989).

Regions within the crystallographic structure of a polypeptide are chosen so as to minimize potential steric hindrance imposed by coupling a relatively large molecule, such as a pIgR binding sFv, to the cysteinyl residue. Any of the amino acids in loops or unstructured regions may be substituted with a cysteine; however, preferred positions exist. Such preferred positions are at amino acids whose side chains are not hydrogen bonded to other amino acid side chains (or backbone atoms) or do not participate or contribute to the formation of salt or charge bridges with other amino acid side chains. Amino acids within helical regions may also be substituted if their side chains are oriented away from the main body of the protein and do not participate in interactions with other amino acid side chains that provide stability to the structure. A preferred position is an amino acid side chain that is fully exposed to bulk solvent and has no significant interaction with amino acids within the polypeptide's tertiary structure and does not participate in the biological activity or function of the molecule, including receptor binding, signal transduction, and the like.

Amino acids for possible substitution may be chosen by examining the crystallographic structure using software designed for the purpose of visualization of the three dimensional structure. Several programs are available for analysis, including Protein Explorer, Insight II, MDL, Tripos, Amber, Charm, Chem-X, Chime, DOCK, Homology, MAGE, SYBYL, Midas Plus, and others known to those skilled in the art. Both visual inspection and calculations and displays within these programs can be used by those skilled in the art to select substitution positions.

A protein or peptide surface is examined for sites at which substitution of an amino acid by cysteine or insertion of cysteine in the protein sequence does not change or modify the activity of the protein in a significant way. Examination of crystallographic data of the protein or peptide will reveal which amino acid residues and side chains are exposed, as judged by the ability of a water molecule to contact the amino acid or its side chain. Cysteine residues are inserted or substituted into loops, preferably loops that are not defined in crystallographic structures because they are so unstructured that they move during data collection. Cysteine residues are substituted for amino acids on the solvent side of helices. Those skilled in the art will know how to use software programs to analyze the surface features of a protein for the purpose of cysteine substitution or insertion (see, e.g., U.S. Pat. Nos. 4,853,871 and 4,908,773).

In some crystal structures the entirety of a loop is not observed because the flexibility of that region has prevented data from being recorded; therefore, the trace of backbone chain terminates as it enters the flexible region and then appears on the other side of the flexible loop. Such regions are suitable for cysteine replacement or insertion. Any amino acid that is in contact with water is a candidate for replacement by cysteine. Such amino acids may be replaced by cysteine, one by one, and the effect of the substitution examined on biological activity. Those substitutions that do not affect biological activity more than 0 to 20 percent, preferably 0 to 10 percent, and most preferably 0 to 5 percent may be used to cross-link to ligands that bind to pIgR and pIgR stalk.

Loops formed by a small stretch of 5 to 15 amino acids on the surface of a protein or peptide are used to insert a cysteine into the protein sequence. Examination of the surface is expected to reveal a site that has maximum exposure to the bulk solvent. Solvent accessible side chains are identified by examining the Connolly (Connolly/Richards) surfaces of the protein, which are essentially defined by the ability of the side chain to contact a water molecule ‘rolled’ around the surface of the molecule. Insertion of a cysteine at a site accessible to bulk solvent, or within 2 to 4 amino acid residues, is performed to produce a variant of the protein suitable for cross-linking to ligands that bind to pIgR or pIgR stalk. Loops are also identified by performing molecular dynamic analysis on the protein. Molecular dynamic analysis carried out over 50 to 250 picoseconds is expected to reveal flexible regions within the structure of the protein that are used for cysteine substitution or insertion. In such analyses, Cysteine residues are substituted, one at a time, between each pair of amino acids in the flexible loop.

Helical wheels are used to identify the side of the helix that faces bulk solvent. Looking down the barrel of a helix, one can identify residues on one side or the other of the helix. Where crystallographic solutions to the protein structure are available, residues on a helical wheel can be observed in the structure to estimate their access to bulk solvent. Residues on the bulk solvent side of the helical wheel often participate in receptor binding. Substitution of a cysteine for such a residue is undesirable. Substitution within a helix at a residue facing the bulk solvent is provided in this invention, provided that the residue does not participate in receptor binding or is otherwise involved in the biological activity of the molecule. Substitution or insertion of cysteine should not alter biological function and activity.

The effect of the cysteine insertion or substitution may be analyzed using biological assays that suitably and appropriately measure the function of the modified protein. Comparisons between the cysteine modified protein and the parent unmodified protein reveal the quantitative and qualitative effects of the modification on function. If data are available that identify, locate, or suggest where the important sites are located on the protein surface that contribute to biological activity, or which cannot be modified by mutagenesis, sites remote for those biologically and functionally sensitive regions may be avoided. For example, the cysteine substitution or insertion may be placed on the surface of the protein or peptide opposite from the functionally sensitive surfaces of the protein; i.e., spatially as far away as possible.

Cysteine substitutions or insertions for antibodies have been described (see U.S. Pat. No. 5,219,996). Methods for introducing Cys residues into the contant region of the IgG antibodies for use in site-specific conjugation of antibodies are described by Stimmel et al. (J. Biol. Chem 275:330445-30450, 2000).

V.B.3. Chemical Addition of Sulfhydryl Groups to Polypeptides and Other Macromolecules

If the desired sulfhydryl groups are not present on the protein, peptide or macromolecule, a sulfhydryl may be introduced by chemical modification. As a nonlimiting example, the sFv or a therapeutic macromolecule can be modified so as to introduce a thiol by chemical modification. A cysteine amino acid can be inserted or substituted on the surface of a protein or peptide by genetic manipulation. Sulfhydryl groups can be added by chemical modification using 2-iminothiolane (IT), also known as Traut's reagent. For example, a sulfhydryl can be introduced by incubating 0.1 to 10 mg/ml, preferably 1 to 5 mg/ml, of the target molecule, with a 1.1- to 100-fold, preferably 1.1- to 10-fold, molar excess of 2-iminothiolane in 50 mM triethanolamine, pH 8.0, containing 150 mM NaCl and 1 mM EDTA for three hours at 4° C. The excess 2-iminothiolane can then be removed by desalting on either a P10 (Bio-Rad, Hercules, Calif.) or G25, G-50, or G-100 (Pharmacia, Piscataway, N.J.) size exclusion column equilibrated with 20 mM sodium phosphate containing 0.15 M NaCl and 1 mM EDTA, pH 7.3, (PBS-EDTA). The selection of either the P10, Sephadex G-25, or Sepharose G-100 columns for desalting is made according to the mass of the derivatized protein.

A protected sulfhydryl group can be added which allows storage of the derivatized protein without self-association through disulfide bond formation. IT and DTNB can be reacted together to form TNB-activated IT, which can then be directly added to the target molecule. Also, substituted IT's can be synthesized (Goff and Carroll, Bioconjugate Chem. 1:381-6, 1990), and using these to add sulfhydryl groups to target proteins can result in disulfide linked conjugates that exhibit increased stability in vivo (Carroll et al. Bioconjugate Chem. 5:248-56, 1994). Protected sulfhydryls can also be added by using a modification reagent that contains a protected thiol in addition to a group that selectively reacts with primary amines. For example, the N-hydroxy-succinimide ester of S-acetylthioacetic acid (SATA, Pierce Chemical Co., Rockford, Ill. can be used according to the manufacturer's instructions to introduce a protected thiol group on either the sFv or the therapeutic macromolecule. This can be accomplished by adding 5 μl of 15 mg/ml SATA in DMSO to 1.0 ml of 60 μM sFv or therapeutic macromolecule in 50 mM sodium phosphate, pH 7.5, containing 1 mM EDTA (P-EDTA). After incubation at room temperature for 30 minutes, the excess SATA can be removed by desalting on either a P10 or G25 size exclusion column equilibrated with P-EDTA. Deprotection of the thiol group can be done by incubating 1 ml of derivatized protein with 100 μl 50 mM sodium phosphate containing 25 mM EDTA and 0.5 M hydroxylamine, pH 7.5, for two hours at room temperature. The excess hydroxylamine can be removed by desalting on either a P10 or G25 size exclusion column equilibrated with PBS-EDTA. Alternatively, one could use N-succinimidyl S-acetylthiopropionate (SATP), which is similar to SATA, but has an additional carbon in the spacer arm. Its use is identical to SATA.

Sulfhydryl groups can also be added by using a modification reagent that contains a disulfide bond in addition to a group that selectively reacts with primary amines. For example, the heterobifunctional cross-linker sulfosuccinimidyl 6-[3′-(2-pyridyldithio)-propionamido] hexanoate (sulfo-LC-SPDP, Pierce Chemical Co.) will thiolate proteins when used according to the manufacturer's directions. Thiolation can also be performed by the addition of 300 μg sulfo-LC-SPDP per ml of 10 mg/ml sFv or therapeutic macromolecule in 20 mM sodium phosphate containing 0.15 M NaCl, pH 7.3 (PBS). Other, non-soluble, forms such as N-succinimidyl 3-(2-pyridyldithio)propionate (SPDP, Pierce Chemical Co.) or N-succinimidyl 6-[3′-(2-pyridyldithio)propionamido]hexanoate (LC-SPDP, Pierce Chemical Co.) can be used in these reactions by dissolving in DMSO to a concentration of 20 mM, and adding 25 μl to 1 ml of 10 mg/ml protein. Reducing the SPDP-derivatized protein under mild conditions will release pyridine-2-thione, leaving an aliphatic thiol. An example of a mild reducing condition is to add 1/100th volume of 1M dithiothreitol (DTT) to the above SPDP-derivatized target protein and incubating for 30 minutes at room temperature, or incubate the SPDP-derivatized target protein with 50 mM 2-meraptoethylamine in PBS-EDTA for 90 minutes at 37° C. The excess SPDP, LC-SPDP or sulfo-LC-SPDP, and the pyridine-2-thione can then be removed by desalting on either a P10 (Bio-Rad, Hercules, Calif.) or G25 (PD10 column, Pharmacia, Piscataway, N.J.) column equilibrated with PBS-EDTA.

These modification reagents may also contain groups near the added thiol such that they form a hindered disulfide when oxidized. These reagents, such as 4-succinimidyloxycarbonyl-methyl-(2-pyridyldithio)-toluene (SMPT), may result in a conjugate that exhibits increased stability in vivo (Thorpe et al. Cancer Res. 47:5924-5931, 1987). Other cross-linking reagents can be used for protein thiolation and are known to those well versed in the art. Many of these reagents are described in the Pierce Chemical Co. catalog, or by Ji (Methods Enzymol. 91:580-609, 1983) and Hermanson (Bioconjugate Techniques, Academic Press, Inc., San Diego, 1-785, 1996).

V.B.4. Chemical Addition of Primary Amine Groups to the Surface of a Polypeptide or Macromolecule

If additional primary amines need to be added to either the sFv or the therapeutic macromolecule, they can be introduced through chemical modification or genetic manipulation. Chemical modification to add primary amines may either be reversible or non-reversible. For example, amination of cysteines can be performed using N-(iodoethyl) Trifluoroacetamide (Aminoethyl-8™, Pierce Chemical Co.) by a reaction in which the iodoalkyl group reacts specifically with sulfhydryl groups, forming a stable thioether bond and releasing free iodine. The trifluoroacetate protecting group can then be hydrolyzed to expose the introduced primary amine. A reversible amination of cysteines can be performed by using, for example, 2-aminoethyl-2′-aminoethanethiosulfonate (Pierce Chemical Co.). The primary amine generated by this compound can be removed by disulfide reducing agents.

V.B.5. Conjugation Between Sulfhydryl Residues

Most commonly, the sFv and therapeutic macromolecule will have either sulfhydryl or primary amines as the targets of the cross-linking reagents, and both sulfhydryl and primary amines can either exist naturally or be the result of chemical modification as described above. When both sFv and therapeutic macromolecule have a reduced sulfhydryl, a homobifunctional cross-linker that contains maleimide, pyridyl disulfide, or α-haloacetyl groups can be used for cross-linking. Examples of such cross-linking reagents include, but are not limited to, bismaleimidohexane (BMH) or 1,4-Di-[3′-(2′-pyridyldithio)propionamido]butane (DPDPB). Alternatively, a heterobifunctional cross-linker that contains a combination of maleimide, pyridyl disulfide, or α-haloacetyl groups can be used for cross-linking. Less preferably, the cross-linking reagent can contain thiophthalimide derivatives or disulfide dioxide derivatives. Also, extrinsic sulfhydryl groups can be introduced into the sFv and therapeutic macromolecule, and oxidized to cross-link by disulfide formation.

V.B.6. Conjugation Between Primary Amines

When primary amines are selected as the target both on sFv and therapeutic macromolecule, then a homobifunctional cross-linker that contains succinimide ester, imidoester, acylazide, or isocyanate groups can be used for cross-linking. Examples of such cross-linking reagents include, but are not limited to, Disuccinimidyl glutarate (DSG), Bis[2-(succinimidooxycarbonyloxy)ethyl]sulfone (BSOCOES), Bis[2-(sulfosuccinimidooxycarbonyloxy)ethyl]sulfone (sulfo-BSCOCOES), Disuccinimidyl suberate (DSS), Dithiobis(succinimidylpropionate) (DSP), BIS-(Sulfosuccinimidyl) Suberate (BS3), Dithiobis(sulfosuccinimidylpropionate) (DTSSP), Disuccinimidyl tartrate (DST), Disulfosuccinimidyl tartrate (sulfo-DST), Dithio-bis-maleimidoethane (DTME), Ethylene glycolbis(succinimidylsuccinate) (EGS), Ethylene glycolbis(sulfosuccinimidylsuccinate) (sulfo-EGS), Dimethyl malonimidate.2 HCl (DMM), Dimethyl suceinimidate.2 HCl (DMSC), Dimethyl adipimidate.2-HCl (DMA), Dimethyl pimelimidate.2 HCl (DMP), Dimethyl suberimidate.2 HCl (DMS), and Dimethyl 3,3′-dithiobispropionimidate.2HCl (DTBP). Heterobifunctional cross-linkers that contains a combination of imidoester or succinimide ester groups can also be used for cross-linking.

V.B.7. Conjugation Between Sulfhydryls and Primary Amines

Heterobifunctional cross-linking reagents that combine selective groups against different targets are generally preferred because these allow reactions to be performed selectively and sequentially, minimizing self-association or polymerization. Also, heterobifunctional reagents allow selection of chemistry appropriate for the individual molecules to be joined, minimizing inhibition of enzymatic, binding, signaling or other activities required for the sFv-therapeutic macromolecule conjugate. For example, some enzymes have a primary amine present in the active site and modification of this amine will inhibit enzymatic function. These enzymes would be suitable prospects for alternative conjugation chemistry so that a thiol group is modified rather than the amine required for therapeutic activity. Examples of such cross-linking reagents include, but are not limited to, N-succinimidyl 3-(2-pyridyldithio)propionate (SPDP), N-succinimidyl 6-[3′-(2-pyridyldithio)propionamido]hexanoate (LC-SPDP), sulfosuccinimidyl 6-[3′-(2-pyridyldithio)-propionamido] hexanoate (sulfo-LC-SPDP), m-maleimidobenzoyl-N-hydoxysuccinimide ester (MBS), m-maleimidobenzoyl-N-hydoxysulfosuccinimide ester (sulfo-MBS), succinimidyl 4-[P-maleimidophenyl] butyrate (SMPB), sulfosuccinimidyl 4-[p-maleimidophenyl] butyrate (sulfo-SMPB), N-[γ-Maleimidobutyryloxy] succinimide ester (GMBS), N-[γ-maleimidobutyryloxy] sulfosuccinimide ester (sulfo-GMBS), N-[ε-maleimidocaproyloxy] succinimide ester (EMCS), N-[ε-maleimidocaproyloxy] sulfosuccinimide ester (sulfo-EMCS), N-succinimidyl(4-iodoacetyl)aminobenzoate (SIAB), sulfosuccinimidyl(4-iodoacetyl)aminobenzoate (sulfo-SIAB), succinimidyl 4-(N-maleimidomethyl)cyclohexane-1-carboxylate (SMCC), sulfosuccinimidyl 4-(N-maleimidomethyl)cyclohexane-1-carboxylate (sulfo-SMCC),succiminidyl-4-(N-maleimidomethyl)cyclohexane-1-carboxy-(6-amido-caproate) (LC-SMCC), 4-succinimidyloxycarbonyl-methyl-(2-pyridyldithio) toluene (SMPT), and sulfo-LC-SMPT.

VI. Methods of Isolation and Purification

After synthesis, it is preferred that a composition or compound isolated or purified, preferably substantially purified. By “isolated” it is meant that the composition or compound has been separated from any molecule that interferes with the biological activity or pIgR-targeting capacity thereof. As used herein the term “substantially purified” means at least about 95%, preferably at least about 99%, free of other components used to produce and/or modify the protein conjugate. The term “purified” refers to a composition or compound that has been separated from at least about 50% of undesirable elements.

VI.A. Purification Elements

Optional protein elements can be incorporated into a fusion protein, which may be a compound of the invention or a member of a protein conjugate of the invention, or which may be comprised in a composition of the invention, and used during its purification and/or preparation. For example, as is discussed in more detail above, a protein member may include a protein purification element such as, for example, a “His tag” (His6). A His-tagged protein member or conjugate thereof can be isolated, or at least partially purified, from a composition that further comprises undesirable compounds by contacting the composition with a column of nickel-plated beads. The His-tagged protein member or conjugate will bind to the nickel plating and will thus be retained in the column; undesirable compounds pass through the column. As is explained above in more detail, various methods may be used to remove the protein purification element from the protein member or conjugate after such steps.

Post-translational modifications to a polypeptide may be created in vitro or in vivo. Various chemical treatments can be used for in vitro modifications of pure or semi-pure proteins; whereas in vivo modifications result from the choice of expression system and host cells. Post-translational modifications include, by way of non-limiting example, glycosylation, cleavage, phophorylation, cross-linking, formation or reduction of disulfide bridges, and the like.

VI.B. Affinity Chromatography

Polypeptides that contain pIgR-derived amino acid sequences that are identical or similar to the epitopes to which sFv molecules that bind pIgR are prepared according to known methods. The epitope-containing polypeptides are covalently coupled to thiol Sepharose (activated thio Sepharose 4B contains a thiol group to which peptides may be attached covalently). A thiol containing peptide is reacted with Ellman's reagent (DTNB) to form a mixed disulfide. The TNB-peptide is separated from 2-nitro-5-thiobenzoic acid by gel sizing column chromatography. The TNB-peptide is reacted with thiol Sepharose to form a mixed disulfide of the peptide covalently bound to the resin.

Alternatively, the peptide is reacted to with the thiol-pyridinium moeity to form a mixed disulfide containing the 2-thiopyridinium actived disulfide bond, which is then used to react with thiol Sepharose to form a covalent disulfide bond with the peptide.

As another example, a maleimido group is placed at the amino or carboxyl terminal of the peptide. The maleimido group on the peptide is reacted with thiol Sepharose to form a thioether bond.

The peptide-Sepharose resin is used to bind an sFv, or other antibody derivative that binds the epitope in pIgR that is recognized by the antibody, or a conjugate comprising such an antibody. Depending on the epitope to which the sFv binds in pIgR, the amino acid sequence may be modified to provide the epitope in an amino acid sequence that inlcudes a residue that may be covalently linked to thiol Sepharose.

In the case of sFv5 and its derivatives (sFv5AF and sFv5AF-Cys), the amino acid sequence of the epitope in pIgR is known, see U.S. patent application Ser. No. ______ (attorney docket No. 18062E-000900US), entitled “Ligands Directed To The Non-Secretory Component, Non-Stalk Region of pIgR and Methods of Use Thereof” filed Mar. 26, 2001 by Mostov et al. The amino acid sequence is, at a minimum, DPRLF. The maximum epitope amino acid sequence is QDPRLF in human and LDPRLF, which suggests that the most amino-terminal residue in the epitope sequence is not required for binding to sFv5.

After the sFv or conjugate has been applied to the column, unreactive material is washed through the column. What remains attached to the column be the sFv's, or sFv-conjugates, that specifically binds to the peptide. The specifically bound sFv or conjugate is separated from the column by low pH (pH 3 to 4) treatment for a brief time (preferably less than 15 minutes and preferably less than 5 minutes), by passing free peptide over the column, or by reducing the covalently bound peptide with DTT or mercaptoethanol. When using a free peptide to obtain elution of the sFv or conjugate, the peptide need only contain the epitope to which the sFv binds or it may contain the same peptide sequence (without the cysteine) used to conjugate to the resin.

For maleimide conjugated peptide to the thiol Sepharose resin, reduction will not release the peptide:sFv or conjugate complex. Therefore, elution with free peptide or low pH may be used.

The sequence within the epitope may be varied such that the interaction is weakened compared to the native epitope. By substituting different amino acids within the sequence, a weaker binding peptide sequence may be identified. Weak binding to the immobilized peptide on thiol Sepharose is used to obtain some retention of sFv and conjugates on the column and to allow nonbinding components to pass straight through the column without binding. Therefore, no strenuous conditions may be required for elution and free peptide may not be required for elution. Tribbick et al. (J. Immunol. Methods 139: 155-166, 1991) have described a similar approach. A weak binding peptide epitope is identified by performing alanine scans on the epitope to identify the amino acid side chains that provide most of the binding specificity and strength.

A peptide epitope is identified using a set of peptides designed to explore all of the binding regions of a protein, a general net. All overlapping peptides of a defined length, homologous with the protein, are synthesised. The offset is set from 1 to 5 residues, and preferably 3 to 4 residues in the first trials. The peptides should be sufficiently long so as not to miss an epitope by ‘dividing it’ between two peptides in the nested set. The peptides should be preferably 8 to 12 amino acids in length and preferably 10 to 15 amino acids in length. The boundaries of the epitope may be more precisely identified using a process that examines the linear sequence of the protein through a series of moving windows of a different size—a window net. The contributions of each amino acid side chain in the epitope are estimated by substituting each amino acid position in the epitope with all of the other 19 amino acids and determining the effect on the binding characteristics of the sFv to the peptide—a replacement net. Such strategies are described by Geysen et al. (Mol. Immunol. 23: 7090715, 1986), Geysen et al. (J. Immunol. Methods 102: 259-274, 1987), Tribbick et al. (J. Immunol. Methods 139: 155-166, 1991), and Geysen et al. (J. Mol. Recog. 1: 32-41, 1988).

VI.C. Ion Exchange Chromatography

In ion exchange chromatography, charged substances are separated via column materials that carry a charge. In cation exchange, the solid phase carries a negative charge whereas, in anion exchange, the stationary phase carries a positive charge. The solid phase of the columns is composed of ionic groups that are covalently bound to a gel matrix. Before a sample is passed through the column, the ionic charges in the solid phase are compensated by small concentrations of counter-ions present in the column buffer. When a sample is added to the column, an exchange occurs between the weakly bound counter-ions in the column buffer and more strongly bound ions present in the sample. Bound molecules do not elute from the column until a solution of varying pH or ionic strength is passed through the column. If desired, the degree of separation may be improved by a change in the gradient slope. If a compound of interest does not bind to the column under the selected conditions, the concentration and/or the pH value of the starting buffer can be changed.

Ion chromatography of polypeptides occurs because polypeptides are multivalant anions or cations. Under strongly basic conditions, polypeptides are anions because the amino group is a free base and the carboxy group is dissociated. Under strongly acidic conditions polypeptides are cations as a result of suppression of the dissociation of the carboxy group and protonation of the amino group. Due to the net charge of the polypeptides it is possible to bind them to a corresponding charged stationary phase as long as the salt concentration is kept low.

Various ion-exchange resins, cellulose derivatives and large-pore gels are available for chromatographic use. Ion-exchange materials are generally water insoluble polymers containing cationic or anionic groups. Non-limiting examples of cation exchange matrices have anionic functional groups such as —SO₃—, —OPO₃— and —COO⁻, and anion exchange matrices may contain the cationic tertiary and quaternary ammonium groups having the general formulae —NHR⁺⁺ and —NR⁺⁺⁺. Proteins become bound by exchange with the associated counter-ions.

For reviews of ion-exchange chromatography, see Bollag, Ion-exchange chromatography, Methods Mol Biol 36:11-22, 1994; Holthuis et al., Chromatographic techniques for the characterization of proteins, Pharm Biotechnol 7:243-99, 1995; and Kent, Purification of antibodies using ion-exchange chromatography, Methods Mol Biol 115:19-22, 1999.

VI.D. Hydrophobic Interaction Chromatography

Separation of polypeptides and other compounds by hydrophobic interaction chromatography (HIC) is based on the hydrophobicity of the compounds presented to the solvents. HIC separates compounds by mechanisms similar to reversed-phase chromatography (RPC) but under gentle reverse salt gradient elution conditions in aqueous buffers. Because no organic solvent is used in HIC, the biological activity of polypeptides and other compounds is more likely to be retained.

HIC involves sequential adsorption and desorption of protein from solid matrices mediated through non-covalent hydrophobic bonding. Generally, sample molecules in a high salt buffer are loaded on the HIC column. The salt in the buffer interacts with water molecules to reduce the salvation of the molecules in solution, thereby exposing hydrophobic regions in the sample molecules which are consequently adsorbed by the HIC column. The more hydrophobic the compound, the less salt needed to promote binding. A decreasing salt gradient may be used to elute samples from the column. As the ionic strength decreases, the exposure of the hydrophilic regions of the molecules increases, and compounds elute from the column in order of increasing hydrophobicity. Sample elution may also be achieved by the addition of mild organic modifiers or detergents to the elution buffer. Non-limiting examples of HIC-immobilized functional groups that can function to separate compounds include octyl groups, such as those on Octyl Sepharose CL4B media from Pharmacia, and propyl groups, such as those on High Propyl media from Baker. Alkoxy, butyl, and isoamyl functional group resins may also be used.

Hydrophilic interaction chromatography (HILIC) separates compounds by passing a hydrophobic or mostly organic mobile phase across a neutral hydrophilic stationary phase, causing solutes to elute in order of increasing hydrophilicity. For example, with neutral peptides one may use 15 mM ammonium formate and reverse organic conditions. Highly charged molecules require low amounts (e.g., 10 mM) of salt for ion suppression, and a slight perchlorate or sulfate gradient (in a high organic solvent concentration) to effect desorption. HILIC has been used successfully with phosphopeptides, crude extracts, peptide digests, membrane proteins, carbohydrates, histones, oligonucleotides and their antisense analogs, and polar lipids.

In hydrophobic-interaction chromatography, compounds of relatively greater hydrophobicity are retained longer on the column relative to those compounds that are more hydrophilic. Conversely, using hydrophilic-interaction chromatography, hydrophilic compounds are retained longer on the column relative to those compounds that are more hydrophobic.

For reviews and exemplary uses of hydrophobic interaction chromatography (HIC), see Ghosh, Separation of proteins using hydrophobic interaction membrane chromatography, J Chromatogr A 923(1-2):59-64, 2001; Queiroz et al., Hydrophobic interaction chromatography of proteins, J Biotechnol 87(2): 143-59, 2001; Arakawa et al., Solvent modulation in hydrophobic interaction chromatography, Biotechnol Appl Biochem 13(2):151-72, 1991; el Rassi et al., Reversed-phase and hydrophobic interaction chromatography of peptides and proteins, Bioprocess Technol 9:447-94, 1990; Kato, High-performance hydrophobic interaction chromatography of proteins, Adv Chromatogr 26:97-115, 1987; Hjerten, Hydrophobic interaction chromatography of proteins, nucleic acids, viruses, and cells on noncharged amphiphilic gels, Methods Biochem Anal 27:89-108, 1981; Ochoa, Hydrophobic (interaction) chromatography, Biochimie 60(1):1-15, 1978; and in Protein Purification, 2d Ed., Springer-Verlag, New York, pgs 176-179 (1988).

For reviews and exemplary uses of hydrophilic interaction chromatography (HILIC), see Zhu et al., Hydrophilic-interaction chromatography of peptides on hydrophilic and strong cation-exchange columns, J Chromatogr 548(1-2):13-24, 1991; Olsen, Hydrophilic interaction chromatography using amino and silica columns for the determination of polar pharmaceuticals and impurities, J Chromatogr A. 913(1-2):113-22, 2001; Olsen, Hydrophilic interaction chromatography using amino and silica columns for the determination of polar pharmaceuticals and impurities, J Chromatogr A. 913(1-2):113-22, 2001; and Alpert et al., Hydrophilic-interaction chromatography of complex carbohydrates, J Chromatogr A. 676(1):191-22, 1994; and Alpert, Hydrophilic-interaction chromatography for the separation of peptides, nucleic acids and other polar compounds, J Chromatogr. 499:177-96, 1990.

VI.E. Assaying Purity and Activity

During or after the purification process, it is often desirable to monitor both the amount and biological activity of the composition, complex or compound being purified. The amount of the composition or compound can be detected by using antibodies directed to an epitope thereof. Additionally or alternatively, a composition or compound of the invention may comprise a detectable polypeptide by which the protein conjugate may be monitored.

Some of the biological activities of a composition or compound of the invention will vary depending on the nature of the biologically active polypeptide(s) included therein, and assays specific for the biological activities of the parent proteins are used. The compositions or compounds are also assayed for their ability to bind pIgR and undergo various forms of cellular trafficking. Assays for these and pIgR-related attributes are described herein and are applicable to any of the compositions or compounds of the invention.

Purity can be assessed by any suitable method, including HPLC analysis and staining of gels through which an aliquot of the preparation containing the protein conjugate has been electrophoresed. Those practiced in the art will know what degree of isolation or purification is appropriate for a given application. For example, (in the U.S. at least) biologicals do not have to meet the same standard of purity for, e.g., a compound.

VII. Multivalent Complexes and Compounds

VII.A. Multivalent Single-Chain Antibodies (Diabodies, Triabodies, etc.)

Diabodies are dimeric antibody fragments (Hollinger et al., “Diabodies”: small bivalent and bispecific antibody fragments, Proc Natl Acad Sci USA Jul. 15, 1993;90(14):6444-8). In each polypeptide, a heavy-chain variable domain V(H) is linked to a light-chain variable domain V(L) but unlike single-chain Fv fragments, each antigen-binding site is formed by pairing of one V(H) and one V(L) domain from the two different polypeptides. Diabodies thus have two antigen-binding sites, and can be bispecific or bivalent. (Perisic et al., Crystal structure of a diabody, a bivalent antibody fragment, Structure Dec. 15, 1994;2(12):1217-26).

VII.A.1 Directing Multimerization of sFv's by Altering Linker Length in sFv Antibodies

The length of the linker(s) between V-domains influences the size, flexibility and valency of single chain Fv antibody fragments (sFv's). sFv molecules are predominantly monomeric when the V(H) and V(L) domains are joined by polypeptide linkers of at least 12 amino acid residues. An sFv molecule with a linker of 3 to 12 amino acid residue is less likely to fold into a monomer, i.e., a single chain Fv in which the V(H) and V(L) domains are paired intramolecularly. However, sFv's that do not easily form monomers may interact with a second sFv molecule to form a “diabody”. Diabodies are bivalent or bispecific. A bivalent diabody is formed from two sFv's that are identical to, or substantially the same as, each other; it has two binding [V(H)::V(L)] regions directed to the same target molecule. A bispecific diabody is formed from two sFv's that are different from each other, and has two binding [V(H)::V(L)] regions, each of which is directed to a different target molecule.

Reducing the linker length below three amino acid residues can force sFv molecules to associate to form multimers (e.g., trimers a.ka. triabodies, tetramers a.k.a., tetrabodies, etc.) depending on linker length, composition and V-domain orientation. The increased valency in sFv multimers may result in higher avidity (low off-rates) (Hudson et al., High avidity scFv multimers; diabodies and triabodies, J Immunol Methods Dec. 10, 1999;231(1-2):177-89; Todorovska et al., Design and application of diabodies, triabodies and tetrabodies for cancer targeting, J. Immunol Methods Feb. 1, 2001;248(1-2):47-66; Hudson et al., High avidity scFv multimers; diabodies and triabodies, J Immunol Methods Dec. 10, 1999;231(1-2):177-89).

Single-chain antibodies having V(H) and V(L) domains with 10-residue (Gly₄Ser)₂ or five-residue (Gly₄Ser) linkers, or no linkers, have been examined. In one report (Kortt et al., Single-chain Fv fragments of anti-neuramimidase antibody NC10 containing five- and ten-residue linkers form dimers and with zero-residue linker a trimer, Protein Engineering, 10:423-433, 1997), the zero-residue linker sFv formed a trimer with three active antigen combining sites. BIAcore biosensor experiments showed that the affinity of each individual antigen combining site in both the 10- and five-residue linker sFv dimers and zero-residue liner sFv trimer was essentially the same when the sFvs were immobilized onto the sensor surface. However, when the sFv was used as the analyte, the dimeric and trimeric sFv's showed an apparent increase in binding affinity due to the avidity of binding the multivalent sFv's.

In general, sFv molecules in which the number of amino acid residues between the V(H) and V(L) domains is 0 to 15 are less likely to form monomers and are more likely to form some type of multimer. When the linker length is 1 or 2 amino acids, trimers and/or other multimers are more likely to form. Linker lengths of 3 to 12 amino acids favor the formation of dimers, where sFv's having linkers of 12 or more more amino acids are more likely to form monomers. These rules are not absolute, however, those skilled in the art can prepare and analyze sFv's with differing linker lengths and test them for the presence of monomers and various multimers.

VII.A.2 Multivalent and Polyspecific Antibody Derivatives

Bivalent and bispecific antibodies and their fragments have immense potential for practical application. Hollinger et al. (“Diabodies”: small bivalent and bispecific antibody fragments, Proc Natl Acad Sci USA Jul. 15, 1993;90(14):6444-8) describe the design of small antibody fragments with two antigen-binding sites. The fragments comprise a heavy-chain variable domain V(H) connected to a light-chain variable domain V(L) on the same polypeptide chain, i.e., V(H)-V(L). By using a linker that is too short to allow pairing between the two domains on the same chain, the domains are forced to pair with the complementary domains of another chain and create two antigen-binding sites (see U.S. Pat. No. 5,837,242). As indicated by a computer graphic model of the dimers, the two pairs of domains can pack together with antigen-binding sites pointing in opposite directions. The dimeric antibody fragments, or “diabodies,” can be designed for bivalent or bispecific interactions. Those with 5- and 15-residue linkers had similar binding affinities to the parent antibodies, but a fragment with the V(H) domain joined directly to the V(L) domain was found to have slower dissociation kinetics and an improved affinity for hapten. Diabodies offer a ready means of constructing small bivalent and bispecific antibody fragments in bacteria.

Higher multimers of sFv molecules may be polyvalent, polyspecific, or both (see, e.g., Müller et al., “A dimeric bispecific miniantibody combines two specificities with avidity”, Federation of European Biochemical Societies, 432 (1998), pp. 45-49). Using triabodies as a non-limiting example of higher multimers of sFv's, it can be seen that there are three possible combinations of sFv molecules. First, a triabody may comprise three identical or substantially identical sFv molecules, each of which is directed to the same target molecule, and is thus a trivalent triabody. Second, a triabody may comprise three different sFv molecules, each of which is directed to a different target molecule, and is thus a trispecific triabody. Third, a triabody may comprise two types of sFv molecules, a pair of which (sFv1a and sFv1b) is directed to a target molecule #1, whereas the third sFv in the triabody is directed to target molecule #2. The latter triabody is both bispecific, as it specifically binds both target molecule #1 and target molecule #2, and bivalent, as it has two binding regions directed to target molecule #1.

VII.A.3. Disulfide-Stabilized Single-Chain Antibodies (dsFv's)

Disulfide-stabilized sFv's (dsFv's) are recombinant Fv fragments of antibodies in which the unstable variable heavy V(H) and variable light V(L) heterodimers are stabilized by disulfide bonds engineered at specific sites that do not appreciably alter the binding activity of the sFv. Such sites lie between structurally conserved framework positions of V(H) and V(L). It should be possible to use positions in conserved framework regions to disulfide-stabilize many different sFv's (Reiter et al., Stabilization of the Fv fragments in recombinant immunotoxins by disulfide bonds engineered into conserved framework regions, Biochemistry May 10, 1994;33(18):5451-9). In addition to influencing the tendency of a sFv molecule to form monomers or multimers, sFv molecules into which Cys residues have been introduced into may in some instances have altered production and stability characteristics.

To improve the stability of Fv molecules, a cysteine residue is introduced into conserved framework regions of both the heavy and light variable domains at positions compatible with the formation of an interdomain disulfide linkage. A disulfide-stabilized Fv (dsFv) may be more resistant to denaturation by heat or urea treatment than the corresponding single-chain Fv (sFv). Moreover, the yield of dsFv may be higher than that of the sFv (Webber et al., Preparation and characterization of a disulfide-stabilized Fv fragment of the anti-Tac antibody: comparison with its single-chain analog, Mol Immunol 1995 March;32(4):249-58; Reiter et al., Antitumor activity and pharmacokinetics in mice of a recombinant immunotoxin containing a disulfide-stabilized sFv fragment, Cancer Res May 15, 1994;54(10):2714-8).

Molecular graphic modeling may be used to identify sites for the introduction of interchain disulfide bonds in the framework region of sFv molecules. Mutations that result in the Cys-modification of the sites are introduced in the reading frame that encodes the sFv molecule using any appropriate method, e.g., PCR-mediated mutagensis. The disulfide-stabilized Fv (dsFv) is expressed and tested for its binding activity (Luo et al., V1-linker-Vh orientation-dependent expression of single chain Fv-containing an engineered disulfide-stabilized bond in the framework regions, J Biochem (Tokyo) 1995 October; 118(4):825-31).

VII.A.4. Production of Multivalent Antibody Derivatives

Technologies that are suitable for the production of multivalent antibody derivatives include F(ab′)₂ assembled from Fab′ fragments expressed in E. coli or isolated by limited proteolysis of a monoclonal antibody; F(ab′)₂ assembled using leucine zippers; and diabodies (Carter et al., Toward the production of bispecific antibody fragments for clinical applications, J Hematother 1995 October;4(5):463-70).

One method for the construction of diabodies uses a refolding system (Takemura et al., Construction of a diabody (small recombinant bispecific antibody) using a refolding system, Protein Eng 2000 August;13(8):583-8). Multivalent disulfide-stabilized sFv's (dsFv's) can be prepared by a variety of methods, including but not limited to phage display (Brinkmann et al., Phage display of disulfide-stabilized Fv fragments, J Immunol Methods May 11, 1995;182(1):41-50). Improved yields of multivalent sFv's may be achieved using the P. pastoris expression/secretion system (Goel et al., Divalent forms of CC49 single-chain antibody constructs in Pichia pastories: expression, purification, and characterization, J. Biochem (Tokyo) 2000 May;127(5):829-36; Powers et al., Expression of single-chain Fv-Fc fusions in Pichia pastoris, J Immunol Methods 251(1-2):123-35, 2001).

Cloning strategies are known that can be used to create repertoires of diabody molecules having two different antigen binding sites (bispecific diabodies) or two of the same, or substantially the same, binding sites (bivalent diabodies) (McGuinness et al., Phage diabody repertoires for selection of large numbers of bispecific antibody fragments, Nat Biotechnol 1996 September;14(9): 1149-54; Pluckthun et al., New protein engineering approaches to multivalent and bispecific antibody fragments, Immunotechnology 1997 June;3(2):83-105); Poljak, Production and structure of diabodies, Structure Dec. 15, 1994;2(12):1121-3; and U.S. Pat. No. 6,071,515).

Phage displaying bivalent diabodies, or multiple copies of sFv monomers, are used to identify multivalent compounds and complexes that bind domain 6, the pIgR stalk, or any other portion or region of pIgR. Phage displaying bivalent diabodies, or multiple copies of sFv monomers, are used to identify multivalent compounds and complexes that are more efficiently endocytosed than phage displaying monomeric sFv. Measurement of phage recovery from within the cytosol as a function of applied phage titer is used to measure the relative endocytotic properties of phage displaying multivalent sFv's (Becerril et al., Toward selection of internalizing antibodies from phage libraries, Biochem Biophys Res Commun Feb. 16, 1999;255(2):386-93). Methods of identifying phage displaying sFv molecules, and other polypeptide sequences, that confer transcytotic and/or paracellular transporting properties are described in U.S. patent application Serial No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., filed Feb. 2, 2001, is drawn to the identification and use of ligands and targeting elements directed to transcytotic and transepithelial molecules.

Multivalent and/or polyspecific compounds and moieties derived from T-cell receptors may also be prepared. See, e.g., Golden et al., High-level production of a secreted, heterodimeric alpha beta murine T-cell receptor in Escherichia coli, J Immunol Methods Aug. 7, 1997;206(1-2):163-9.

VII.B. Fusion Proteins

VII.B.1. Fusion Proteins Comprising Repeats of sFv Sequences

To increase the valency of fusion proteins of the invention, one or more tandem repeats of the DNA sequence that encode the [V(H)-V(L)] domains are introduced into the chimeric reading frame that encodes the fusion protein. For example, in the case of a dimer, two copies of each antibody variable domain, V(H) and V(L), are combined in a single chain construct (see, e.g., U.S. Pat. No. 6,121,424). After expression in E. coli, intramolecularly folded bivalent diabodies are prepared, preferably from soluble periplasmic extracts. The relative amounts of intramolecular diabodies, as opposed to intermolecular tetrabodies formed from the association of V(H) and V(L) domains from two separate diabodies, is dependent on the length of the linker in the middle of the chain and bacterial growth conditions (Kipriyanov et al., Bispecific tandem diabody for tumor therapy with improved antigen binding and pharmacokinetics, J Mol Biol Oct. 15, 1999;293(1):41-56).

Fusion proteins comprising tetravalent single-chain antibodies, e.g., {[V(H)-V(L)]₂}₂ wherein each V(H) and V(L) can combine to form a sFv, may be prepared using similar strategies (Booth et al., Genetically Engineer Tetravalent Single-Chain Fv of the Pancarcinoma Monoclonal Antibody CC49: Improved Biodistribution and Potential for Therapeutic Application, Cancer Research 60, 6964-6971, Dec. 15, 2000). See also U.S. Pat. Nos. 5,869,620; 5,877,291; and 5,892,020.

In fusion proteins comprising single chain Fv (sFv) fragments, the orientations of the V(H) and V(L) domains relative to each other, and other fusion protein elements, influences the expression and activity of the sFv portion (Luo et al., Vl-linker-Vh orientation-dependent expression of single chain Fv-containing an engineered disulfide-stabilized bond in the framework regions, J Biochem (Tokyo) 1995 October; 118(4):825-31).

VII.B.2. Fusion Proteins Comprising Other Targeting Elements

Multivalent fusion proteins can comprise other polypeptidic targeting elements. For example, fusion proteins may comprise polypeptides derived from bacterial proteins that bind to pIgR and/or pIgR stalk molecules (see Example 3). Derivatives of monoclonal antibodies directed to pIgR and/or pIgR stalk molecules, e.g., complementary determining sequences (CDR), (Fab)2 molecules and the like, may be prepared and incorporated into fusion proteins.

VI.C. Other Methods for Multimerization

VII.C.1. Chemical Bonds

Cysteine and other reactive amino acid residues that are naturally present or artificially introduced into a monomer molecule may be reacted in order to create chemically linked multimers. In the case of Cys residues, intermolecular disulfide (—S—S—) bonds may be formed to link monomers together. Such intermolecular disulfide bridges may be eliminated or reduced by addition of reducing agents, e.g., DTT. Chemical cross linkers, e.g., bifunctional linkers, can be used to form chemical bonds between monomers.

Thus, by way of non-limiting example, multivalent compounds may be prepared by the chemical linkage of two monovalent molecules, each of which comprises a targeting element. The multivalent conjugate may then be covalently or non-covalently associated with a bioactive molecule. As another example, multivalent bioactive compounds may be prepared by chemically conjugating two monovalent bioactive molecules (i.e., molecules comprising a bioactive moiety and a single targeting element) to each other. This is one way in which the ratio of bioactive molecules to targeting elements may be controlled; in the former case, the conjugate has 2 targeting elements and 1 bioactive moiety, whereas the latter conjugate comprises 2 targeting elements and 2 bioactive moieties.

VII.C.2. Leucine Zippers

A number of eukaryotic transcription factors comprise a dimerization motif called the “leucine zipper”. These leucine zipper proteins form homodimers and/or heterodimers with another protein containing a leucine zipper motif. Proteins that dimerize due to the presence or introduction of leucine zippers are said to be “leucine zipped.” See, Rieker et al., Molecular applications of fusions to leucine zippers, Methods Enzymol 2000;328:282-96; Hoyne et al., High affinity insulin binding by soluble insulin receptor extracellular domain fused to a leucine zipper, FEBS Lett Aug. 11, 2000;479(1-2):15-8; Behncken et al., Growth hormone (GH)-independent dimerization of GH receptor by a leucine zipper results in constitutive activation, J Biol Chem Jun. 2, 2000;275(22): 17000-7; Busch et al., Dimers, leucine zippers and DNA-binding domains, Trends Genet 1990 February;6(2):36-40; Riley et al., Multimer formation as a consequence of separate homodimerization domains: the human c-Jun leucine zipper is a transplantable dimerization module, Erratum in: Protein Eng 1996 September;9(9):831 Protein Eng 1996 February;9(2):223-30; Schmidt-Dorr et al., Construction, purification, and characterization of a hybrid protein comprising the DNA binding domain of the LexA repressor and the Jun leucine zipper: a circular dichroism and mutagenesis study, Biochemistry Oct. 8, 1991;30(40):9657-64; Dmitrova et al., A new LexA-based genetic system for monitoring and analyzing protein heterodimerization in Escherichia coli, Mol Gen Genet 1998 January;257(2):205-12. Granger-Schnarr et al., Transformation and transactivation suppressor activity of the c-Jun leucine zipper fused to a bacterial repressor, Proc Natl Acad Sci USA May 15, 1992;89(10):4236-9. Methods for preparing leucine-zipped multivalent sFv's have been described; de Kruif, Leucine zipper dimerized bivalent and bispecific scFv antibodies from a semi-synthetic antibody phage display library, J Biol Chem Mar. 29, 1996;271(13):7630-4.

VII.C.3. Other Dimerization Domains

Coiled coil dimerization is described by Willcox et al. (Production of soluble alphabeta T-cell receptor heterodimers suitable for biophysical analysis of ligand binding, Protein Sci 1999 November;8(11):2418-23).

The use of Protein A interactions with immunoglobulins to cause the dimerization of proteins has been described (De A et al., Use of protein A gene fusions for the analysis of structure-function relationship of the transactivator protein C of bacteriophage Mu, Protein Eng 1997 August;10(8):935-41).

GST sequences can be used as dimerization sequences. Tudyka, Glutathione S-transferase can be used as a C-terminal, enzymatically active dimerization module for a recombinant protease inhibitor, and functionally secreted into the periplasm of Escherichia coli, Protein Sci 1997 October;6(10):2180-7.

VII.C.2. Combinations

Any possible combination of covalent and non-covalent bonds may be used to generate the multivalent complexes of the invention. A fusion protein, in which multiple V(H) regions are covalently bonded, may be non-covalently associated with a second fusion protein having multiple V(L) regions that are covalently linked to each other (see, e.g., U.S. Pat. No. 6,239,259), a complex that has the structure found in the following diagram.

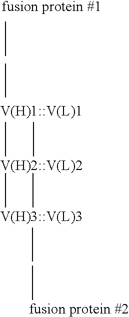

VIII. Calcitonin Polypeptides

Calcitonin is a polypeptide hormone that is primarily produced and secreted by the parafollicular cells of the thyroid gland in mammals and by the ultimobranchial gland of birds and fish, but is also synthesized in a wide variety of other tissues, including the lung and intestinal tract.

Calcitonin is a hormone known to participate in calcium and phosphorus metabolism. Calcitonin controls the activity of osteoclasts (the cells that break down old and weakened bone), so it can be replaced by new bone. It has been shown that the calcitonins reduce calcium concentration in blood (Hirsch et al., Science Vol. 146, page 412, 1963), and inhibit feeding (Freed et al., Science Vol. 206, page 850, 1979) and gastric secretion (Hesch et al., Horm. Metab. Res. Vol. 3, page 140 (1971).

VIII.A. Therapeutic Uses of Calcitonin

Calcitonin inhibits bone resorption through binding and activation of a specific calcitonin receptor on osteoclasts (The Calcitonins-Physiology and Pharmacology Azria (ed.), Karger, Basel, Su., 1989), with a resultant decrease in the amount of calcium released by bone into the serum. This inhibition of bone resorption has been exploited, for instance, by using calcitonin as a treatment for osteoporosis, a disease characterized by a decrease in the skeletal mass often resulting in debilitating and painful fractures, and prevention of fracture in osteogenesis imperfecta.

Calcitonin is also used in the treatment of Paget's disease where it provides rapid relief from bone pain, which is frequently the primary symptom associated with this disease. This analgesic effect has also been demonstrated in patients with osteoporosis or metastatic bone disease and has been reported to relieve pain associated with diabetic neuropathy, cancer, migraine and post-hysterectomy. Reduction in bone pain occurs before the reduction of bone resorption.

Other uses of calcitonin include but are not limited to treatment of hypercalcemia and Paget's disease, counteracting vasospasms, ischemia, renal failure, and treating male impotence.

VIII.B. Calcitonin Structure

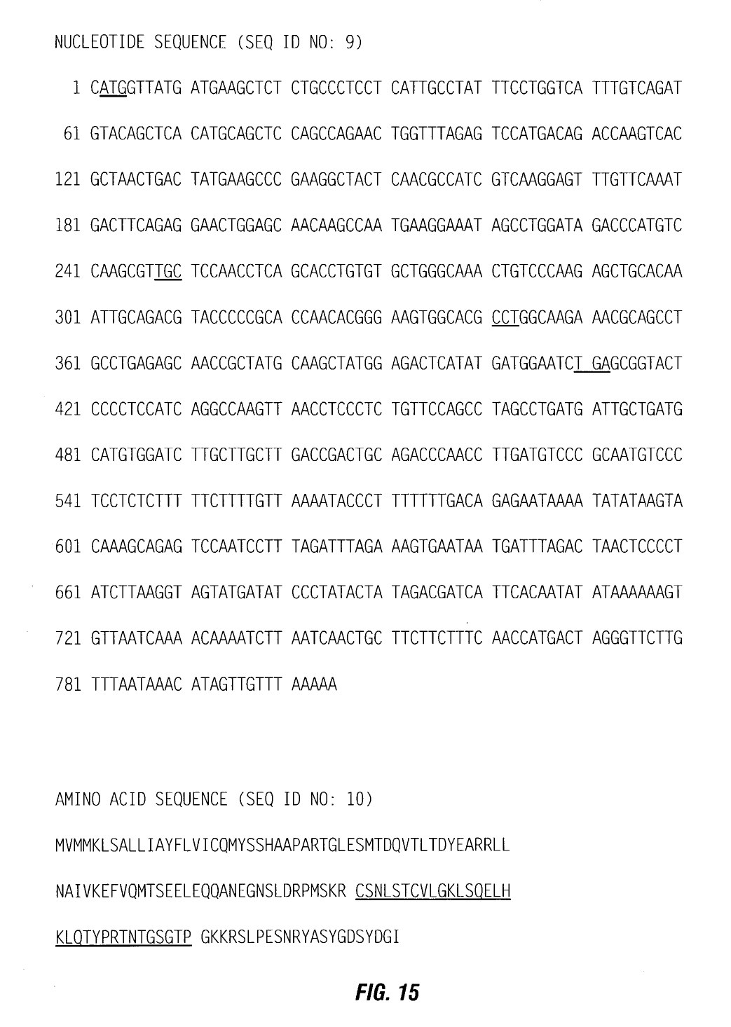

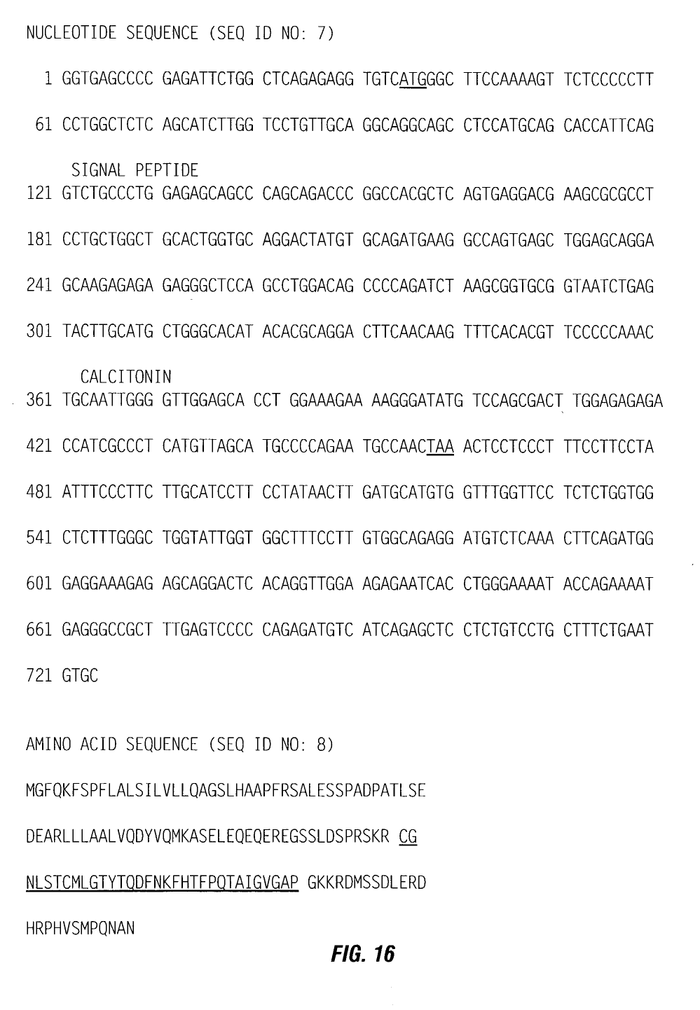

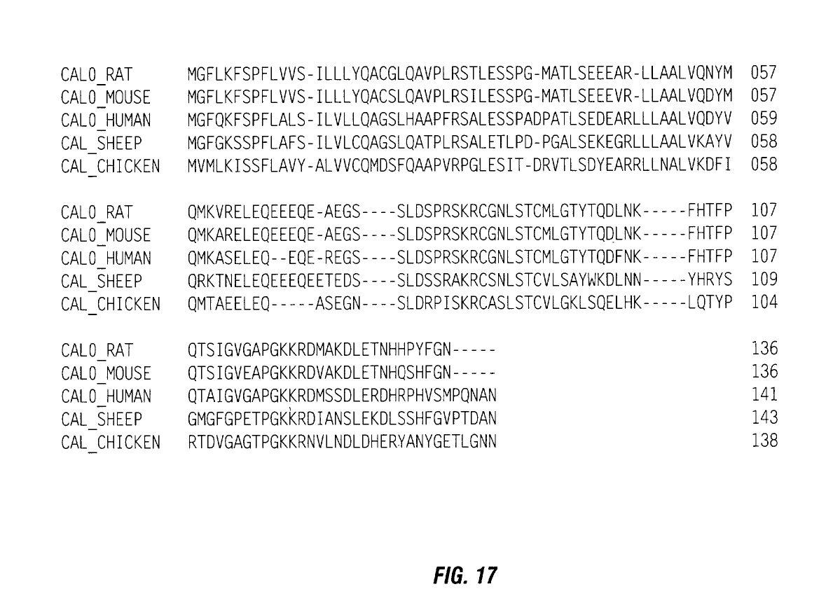

Structural features of calcitonins include a constant chain length of 32 amino acids, a disulfide bridge between the cysteine residues in positions 1 and 7, forming a ring of seven amino acid residues at the N-terminal, and a carboxy terminal proline amide. Amino acid residues common to all calcitonins are those in the 1st, 4th-7th, 28th and 32nd positions (see FIGS. 15, 16 and 17). Thus, full length calcitonins may be characterized for example by a bridge generally between positions 1 and 7 of the polypeptide chain and, alternatively or additionally, by a leucine residue in position 9, and/or a glycine residue in position 28 and/or a proline residue in position 32.

Although not wishing to be bound by any particular theory, it is thought that the proline amide at C-terminal, common to all calcitonins, is indispensable for the biological activities thereof (Potts et al., Calcium, Parathyroid Hormone and the Calcitonins, page 121 printed by Excerpta Medica, Amsterdam (1971); Sieber, Calcitonin 1969, page 28, Proc. 2nd Symp., printed by Medical Books, London (1970); and Rittel et al., Experientia, Vol. 32, page 246 (1976).

VIII.C. Testing of the Biological Activity of Calcitonin Polypeptides and Derivatives

The term calcitonin polypeptide embraces calcitonin derivatives having one or more biological activities of calcitonin. A variety of methods are known in the art that may be used to evaluate the biological activity of calcitonin derivatives. For example, the hypocalcemic rat model can be used to determine the effect of synthetic calcitonin mimetics on serum calcium, and the ovariectomized rat or mouse can be used as a model system for osteoporosis. Bone changes seen in these models and in humans during the early stages of estrogen deficiency are qualitatively similar. Calcitonin has been shown to be an effective agent for the prevention of bone loss in ovariectomized humans and also in rats (Mazzuoli, et al., Calcif. Tissue Int. 47:209-14, 1990; Wronski, et al., Endocrinology 129:2246-50, 1991).

Calcitonin acts directly on osteoclasts via a cell surface receptor, the calcitonin receptor (CRE). CRE is a member of the G-protein receptor family and transduces signal via activation of adenylate cyclase, leading to elevation of cellular cAMP levels (Lin, et al., Science 254:1022-4, 1991). Calcitonin-mediated receptor activation can be detected by: (1) measurement of adenylate cyclase activity (Salomon, et al., Anal. Biochem. 58:541-8, 1974; Alvarez and Daniels, Anal. Biochem. 187:98-103, 1990); (2) measurement of change in intracellular cAMP levels using conventional radioimmunoassay methods (Steiner, et al., J. Biol. Chem. 247:1106-13, 1972; Harper and Brooker, J. Cyc. Nucl. Res. 1:207-18, 1975); or (3) use of a cAMP scintillation proximity assay (SPA) method (Amersham Corp., Arlington Heights, Ill.).

VIII.D. Calcitonin Derivatives

Derivatives of calcitonins include but are not limited to calcitonin structures that have been altered relative to the natural protein, e.g., (a) one or more amino acid radicals are replaced by one or more other amino acid radicals (natural or synthetic) and/or (b) the disulfide bridge is replaced by an alkylene bridge and/or is open, and/or (c) one or several amino acid radicals are omitted (desaminoacyl derivatives).

Calcitonin homologs, i.e, polypeptides derived from non-human species that have amino acid sequences that are related to, but different from, the sequence of human calcitonin, are also calcitonin derivatives within the scope of the invention. Non-limiting examples of calcitonin homologs are listed in Table 9 and described in FIGS. 15 to 17. One skilled in the art will be able to select the appropriate source of DNA, sequence of primers, PCR conditions, etc. for each particular genetic sequence encoding an calcitonin homolog to generate other calcitonin fusion proteins.

TABLE 9
CALCITONIN HOMOLOGS AND ANALOGS

| | Accession | |
|---|---|---|
| Organism(s) | Number(s) | Citation(s) |
| Tobacco hornworm | | Reagan, J. Biol. Chem. 269: 9-12 (1994) |
| Cockroach | | Furuya et al., Proc. Natl. Acad. Sci. U.S.A |
| | | 97: 6469-74 (2000) |
| Bony fishes | | Suzuki et al., Gen. Comp. Endocrinol. |
| (lungfish, sturgen, etc.) | | 113: 369-73 (1999) |
| Cartilaginous fish | | |
| (stingray) | | |
| Teleosts (eels) | | Suzuki et al., Gen. Comp. Endocrinol. |
| | | 113: 369-73 (1999); Hashimoto et al., |
| | | Biochemistry 38: 8366-84 (1999) |
| Reptiles (turtle, snake, | | Suzuki et al., Zoolog. Sci. 14: 833-6 (1997) |
| grass lizard and | | |
| oaimon) | | |
| Salmon | | Stevenson, J. Pept. Res. 55: 129-39 (2000); |
| | | Hong et al., Biochem. Biophys. Res. Commun. |
| | | 267: 362-7 (2000) |
| | | Recombinant production formulated for |
| | | implants |
| Human/Salmon hybrid | GI/2173732 | Takahashi et al., Peptides 18: 439-44 (1997); |
| genes | | Miyake, et al., Patent: JP 1993255391-A 4 |
| | | October 5, 1993 |
| Flounder | GI/2173730 | Suzuki et al., Gene 244: 81-8 (2000) |
| Chicken | GI/222801 | Minvielle et al., FEBS Lett. 203: 7-10 (1986) |
| Fish Calcitonin | GI/2169307 | Narishima et al., Patent: JP 1986291598-A 2; |
| Derivative | GI/2169306 | Dec. 22, 1986 |

Calcitonin analogs are also calcitonin derivatives are also within the scope of the invention. Such analogs may be, for example, polypeptides having amino acid sequences derived from a calcitonin protein. Calcitonin genes and proteins that are the combination of calcitonin sequences from one species to another are also calcitonin analogs as that term is used herein. An example of the latter type of calcitonin analog is one which has an amino-terminal human calcitonin precursor fused to a salmon calcitonin (Takahashi et al., Peptides 18:439-444, 1997). See also U.S. Pat. Nos. 6,265,534; 6,251,635; 6,124,299; 6,107,277; 5,977,298; 5,962,270; 5,710,244; and 5,541,159.

Hybrid or chimeric calcitonin polypeptides may also be prepared and calcitonin derivatives within the scope of the invention. See, e.g., Takahashi et al., Peptides 18:439-44 (1997); U.S. Pat. No. 5,831,000; and Japanese Patent JP 1993255391-A 4.

Various formulations may be preferred for calcitonin delivery depending on the mode of delivery and targeted tissue. See, e.g., U.S. Pat. Nos. 6,149,893; 6,087,338; 5,912,014; 5,726,154; 5,719,122; 5,571,788;, 5,514,365; and Serres, et al., “Temperature and pH-sensitive Polymers for Human Calcitonin Delivery”, Pharmaceutical Research 13:196-201, 1996.

IX. Pharmaceutical Compositions and Therapeutic Methods

Another aspect of the invention is drawn to compositions, including but not limited to pharmaceutical compositions. According to the invention, a “composition” refers to a mixture comprising at least one carrier, preferably a physiologically acceptable carrier, and one or more compositions or compounds of the invention. The term “carrier” defines a chemical compound that does not inhibit or prevent the incorporation of the compositions or compounds into cells or tissues. A carrier typically is an inert substance that allows an active ingredient to be formulated or compounded into a suitable dosage form (e.g., a pill, a capsule, a gel, a film, a tablet, a microparticle (e.g., a microsphere), a solution; an ointment; a paste, an aerosol, a droplet, a colloid or an emulsion etc.). A “physiologically acceptable carrier” is a carrier suitable for use under physiological conditions that does not abrogate (reduce, inhibit, or prevent) the biological activity and properties of the composition or compound of the invention. For example, dimethyl sulfoxide (DMSO) is a carrier as it facilitates the uptake of many organic compounds into the cells or tissues of an organism. Preferably, the carrier is a physiologically acceptable carrier, preferably a pharmaceutically or veterinarily acceptable carrier, in which the composition or compound of the invention is disposed.

A “pharmaceutical composition” refers to a composition wherein the carrier is a pharmaceutically acceptable carrier, while a “veterinary composition” is one wherein the carrier is a veterinarily acceptable carrier. The term “pharmaceutically acceptable carrier” or “veterinarily acceptable carrier” includes any medium or material that is not biologically or otherwise undesirable, i.e, the carrier may be administered to an organism along with a composition or compound of the invention without causing any undesirable biological effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated by reference herein into the present application. The terms “therapeutically effective amount” or “pharmaceutically effective amount” mean an amount sufficient to induce or effectuate a measurable response in the target cell, tissue, or body of an organism. What constitutes a therapeutically effective amount will depend on a variety of factors which the knowledgeable practitioner will take into account in arriving at the desired dosage regimen.

IX.A. Composition of Pharmaceutical Compositions

The pharmaceutical compositions of the invention can further comprise other chemical components, such as diluents and excipients. A “diluent” is a chemical compound diluted in a solvent, preferably an aqueous solvent, that facilitates dissolution of the chimeric pIgR-targeting protein in the solvent, and it may also serve to stabilize the biologically active form of the chimeric pIgR-targeting protein or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-cellulose, sodium carboxymethylcellulose, and/or polyvinylpyrrolidone (PVP). If desired, disintegrating agents can also be included, such as cross-linked polyvinylpyrrolidone, agar, or alginic acid or a salt thereof such as sodium alginate. Other suitable excipients and carriers include hydrogels, gellable hydrocolloids, and chitosan. Chito san micro spheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

IX.B. Formulation of Pharmaceutical Compositions

The compositions and compounds of the invention can be formulated in any suitable manner. The compositions or compounds may be uniformly (homogeneously) or non-uniformly (heterogenously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a pharmaceutical composition according to the invention is compressed into tablet or pill form suitable for oral administration or compounded into a sustained release formulation. When the pharmaceutical composition is intended for oral administration but the composition or compound of the invention is to be delivered to epithelium in the intestines, it is preferred that the formulation be encapsulated with an enteric coating to protect the formulation and prevent premature release of the chimeric pIgR-targeting proteins included therein. As those in the art will appreciate, the pharmaceutical compositions of the invention can be placed into any suitable dosage form. Pills and tablets represent some of such dosage forms. The pharmaceutical compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some preferred embodiments concern compositions that comprise a bioadhesive, preferably a mucoadhesive, coating. A “bioadhesive coating” is a coating that allows a substance (e.g., a composition or chimeric pIgR-targeting protein according to the invention) to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles (e.g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the compositions or compounds of the invention interact with the target cell surface transport moiety.

IX.C. Administration of Pharmaceutical Compositions

The pharmaceutical compositions of the invention facilitate administration of monoclonal antibodies to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, rectal (e.g., an enema or suppository) aerosol (e.g., for nasal or pulmonary delivery), parenteral, and topical administration. Preferably, sufficient quantities of the composition or compound of the invention are delivered to achieve the intended effect. The particular amount of composition or compound to be delivered will depend on many factors, including the effect to be achieved, the type of organism to which the composition is delivered, delivery route, dosage regimen, and the age, health, and sex of the organism. As such, the particular dosage of a composition or compound of the invention included in a given formulation is left to the ordinarily skilled artisan's discretion.

Those skilled in the art will appreciate that when the pharmaceutical compositions of the present invention are administered as agents to achieve a particular desired biological result, which may include a therapeutic or protective effect(s) (including vaccination), it may be necessary to combine the composition or compound of the invention with a suitable pharmaceutical carrier. The choice of pharmaceutical carrier and the preparation of the composition or compound as a therapeutic or protective agent will depend on the intended use and mode of administration. Suitable formulations and methods of administration of therapeutic agents include those for oral, pulmonary, nasal, buccal, ocular, dermal, rectal, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, areosol, droplet, or spray. Pills, tablets, suppositories, areosols, powders, droplets, and sprays may have complex, multilayer structures and have a large range of sizes. Aerosols, powders, droplets, and sprays may range from small (1 micron) to large (200 micron) in size.

Pharmaceutical compositions of the present invention can be used in the form of a solid, a lyophilized powder, a solution, an emulsion, a dispersion, a micelle, a liposome, and the like, wherein the resulting composition contains one or more of the compounds of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695).

The active compound (i.e, a composition or compound of the invention) is included in the pharmaceutical composition in an amount sufficient to produce the desired effect upon the process or condition of diseases.

IX.D. Uses of Pharmaceutical Compositions

The pharmaceutical compositions of the invention facilitate administration of monoclonal antibodies to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a pharmaceutical composition exist in the art including, but not limited to, oral, aerosol (e.g., for nasal or pulmonary delivery), parenteral, and topical administration. Preferably, a sufficient quantity of the Mab portion of the pharmaceutical composition is delivered to achieve the intended effect. The particular amount of Mab to be delivered will depend on many factors, including the effect to be achieved, the type of organism to which the pharmaceutical composition is delivered, delivery route, dosage regimen, and the age, health, and sex of the organism. As such, the particular dosage of composition or compound of the invention included in a given formulation is left to the ordinarily skilled artisan's discretion.

In another therapeutic context, the pharmaceutical compositions of the invention allow a Mab to be efficaciously delivered as part of a pIgR-targeting composition or compound. Because pIgR-ligands are delivered into cells by active transport, the instant pharmaceutical compositions afford better control over bioavailability of monoclonal antibodies as compared to passive transport mechanisms. As such, the pIgR-targeting protein conjugates and compositions of the invention enable improved uptake and utilization of the monoclonal antibody.

The compositions and compounds of the invention are also useful in diagnostic and related applications. One aspect of the invention involves the diagnosis and monitoring of certain diseases, preferably in kit form. This aspect is useful for assaying and monitoring the course of the diagnosis and prognosis of disease, for monitoring the effectiveness and/or distribution of a therapeutic agent or an endogenous compound, in a patient as well as other related functions.

In this aspect of the invention, it may be desirable to monitor or determine if, or determine the degree to which, a patient's pIgR-displaying cells are capable of, or presently are, endocytosing a detectably labeled composition or compound of the invention. Such methods are used in a variety of systems depending on the nature of the pIgR-targeting element(s) of a given protein conjugate.

For example, the degree to which a patient, or a biological sample therefrom, endocytoses a composition or compound that has a pIgR-targeting element derived from a bacterial protein that binds pIgR is a measure of a patient's susceptibility to infection by bacteria having that element. A higher degree or rate of uptake of the detectable label indicates that the patient is more susceptible to such infection.

As another example, the activity, distribution and/or concentration of endogenous pIgR proteins may be altered in various ways during the course of a disease or disorder. The pIgR proteins in a patient are measured over the course of a disease for diagnostic and prognostic purposes, as well as over the course of treatment of a disease or disorder, in order to monitor the effects on pIgR proteins. Diseases to which this aspect of the invention can be applied include but are not limited to diseases that involve the respiratory system, such as lung cancer and tumors, asthma, pathogenic infections, allergy-related disorders, and the like; the gastrointestinal tract, including cancers, tumors, pathogenic infections, disorders relating to gastroinstestinal hormones, Chron's disease, eating disorders, and the like; and any disease or disorder that is known or suspected to involve pIgR-displaying cells.

Compositions and compounds of the invention may be detectably labeled by virtue of comprising a detectable polypeptide such as, e.g., a green fluorescent protein (GFP) or a derivative thereof. If the protein conjugate comprises an epitope for which antibodies are available (including but not limited to commercially available ones such as c-myc epitope and the FLAG-tag), it may be detected using any of a variety of immunoassays such as enzyme-linked immunosorbent assay (ELISA) or a radioimmunoassay (RIA).

### EXAMPLES

### Example 1

### Molecular Reagents

1.1 Preparation of a Polyclonal Anti-5AF-Cys Antibody

In the Examples, polyclonal antibodies directed to sFv5AF are used to simultaneously detect the single-chain antibodies sFv5AF and sFv5AF-Cys, and conjugates comprising these sFv's. The anti-sFv5AF polyclonal antibodies were prepared as follows.

FLAG-tagged sFv5AF was used as an immunogen for the production of antisera (polyclonal antibodies). The antisera was commercially prepared by HTI Bio-Products (Ramona, Calif.). In brief, 200 μg of FLAG-tagged sFv5AF was used for the initial injection (Day 1) with Complete Freund's Adjuvant, followed by boosts of 200 μg fusion protein with Incomplete Freund's Adjuvant every 2 weeks. The injections were subcutaneous. Bleeds were taken at approximately 7 weeks and 9 weeks.

The sera was screened for reactivity with sFv5AF using an ELISA. Sera that tested positive in the ELISA were examined by Western blot to confirm the presence of polyclonal antibodies reactive with sFv5AF.

1.2. Preparation of a Rabbit/Rat Chimeric pIgR

Expression of pIgR in Madin-Darby canine kidney (MDCK) cells using retroviral vectors has been described by Breitfeld et al. (Methods Cell Biol 32:329-37, 1989). The expression of human pIgR in MDCK cells has been described by Tamer et al. (J. Immunol 1995 155:707-14, 1995). Because rats are useful for in vivo assays, initial in vitro transcytosis assays used MDCK cells transfected with rat pIgR. However, the expression of rat pIgR in transfected MDCK cells was reduced relative to results obtained with rabbit pIgR transfected MDCK cells. Without wishing to be bound by any particular theory, the relatively reduced expression of rat pIgR may be a consequence of an unusual structure in the 5′ untranslated region of the rat pIgR cDNA (Fabregat et al., Physiol Genomics 5:53-65, 2001; Fodor et al., DNA Cell Biol 16:215-25, 1997; Koch et al., Nucleic Acids Res 23:1098-112, 1995).

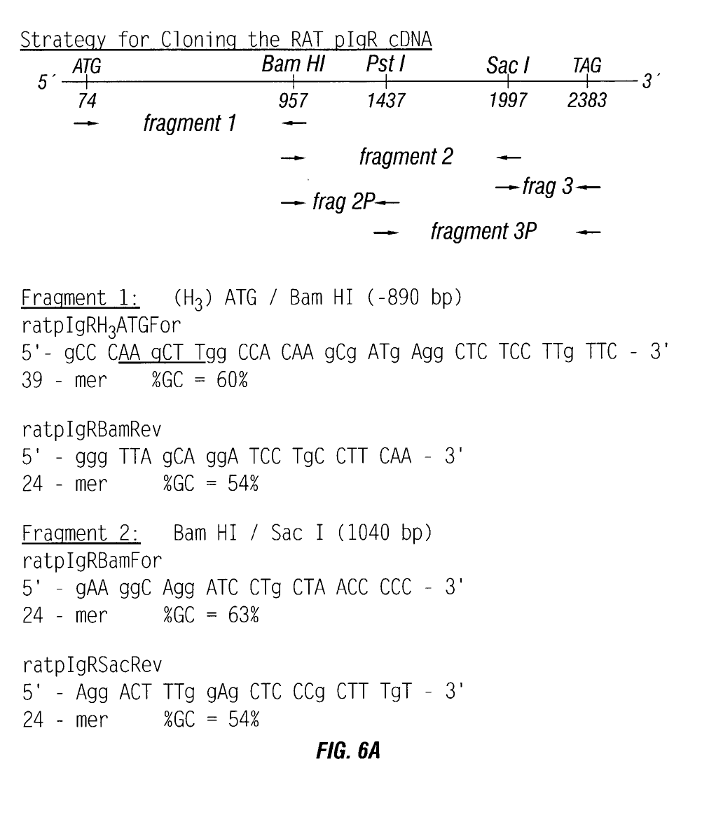

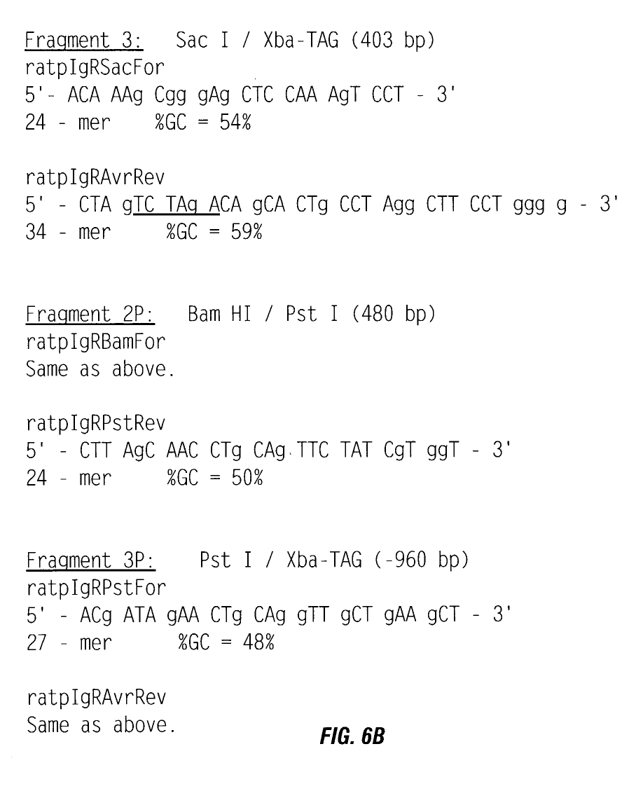

To enhance the production of a rat-like pIgR in transfected MDCK cells, a chimeric protein was produced via PCR using primers to rat and rabbit pIgR cDNA sequences and methods known in the art. The chimeric protein consists of amino acids 1-554 of rabbit pIgR, followed by amino acids 553-645 of rat pIgR, then amino acids 651-756 of rabbit pIgR. This chimeric protein contains the stalk and transmembrane regions of rat pIgR, and the remainder of the molecule is derived from rabbit pIgR. The chimeric pIgR has the same activity as wild type rabbit pIgR in pIgR assays such as transcytosis of IgA from the basolateral to the apical surface (forward transcytosis). The structure and amino acid sequence of the chimeric pIgR protein are shown in FIGS. 6A and 6B, respectively. The chimeric protein was expressed from an expression construct comprising the expression vector pCB7.

### Example 2

### Cloning of pIgR Genes from Various Animals and Chimeric Rat/Rabbit pIgR

2.1. Cloning of Rat pIgR cDNA

A rat liver cDNA library (Clontech) was used as a source for template for the amplification of rat pIgR sequences. The pIgR cDNA was amplified as 5 separate fragments which can be combined to regenerate the entire rat pIgR sequence (see FIG. 6). Alternatively, the sequences contained within separately cloned cDNA's may be used as a source for sequences that encode a mouse stalk molecule or sequences derived therefrom.

As can be seen in FIG. 6, the primers used to amplify the rat cDNA regenerated or introduced restriction enzyme sites into the cDNA for ease of subcloning and other subsequent manipulations. Each fragment was treated with the appropriate restriction enzymes and ligated into a cloning vector (e.g., pBluescript from Stratagene or pUC19 from NEB) in order to generate an “intermediate vector”. The sequence of the inserted cDNA was determined in order to confirm the sequence of the amplified DNA.

2.2. Cloning of Mouse pIgR cDNA

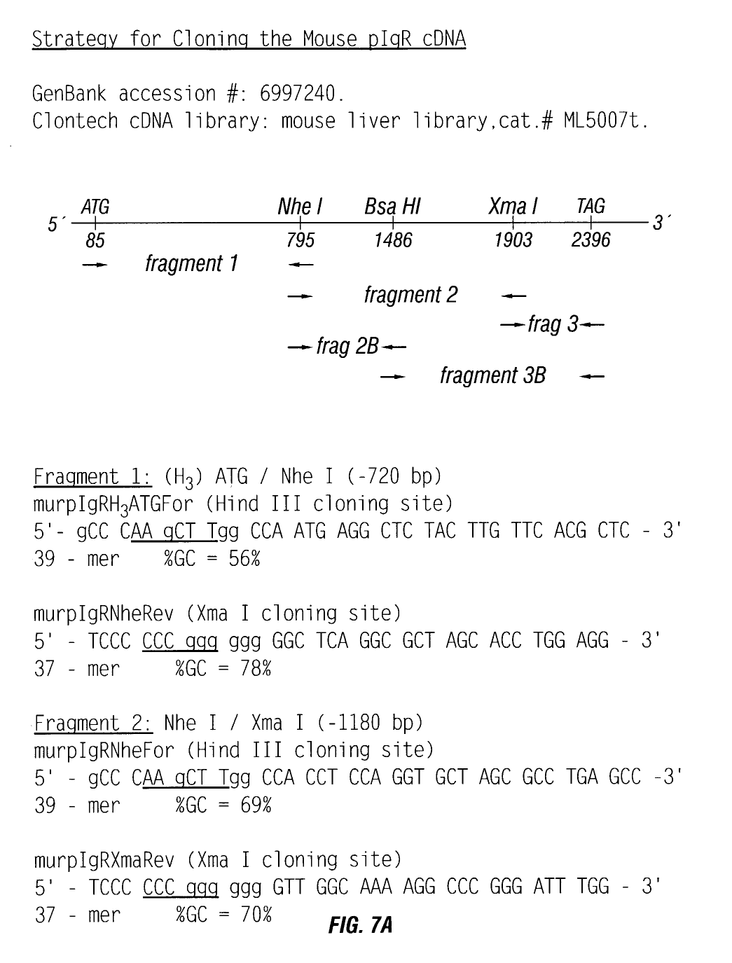

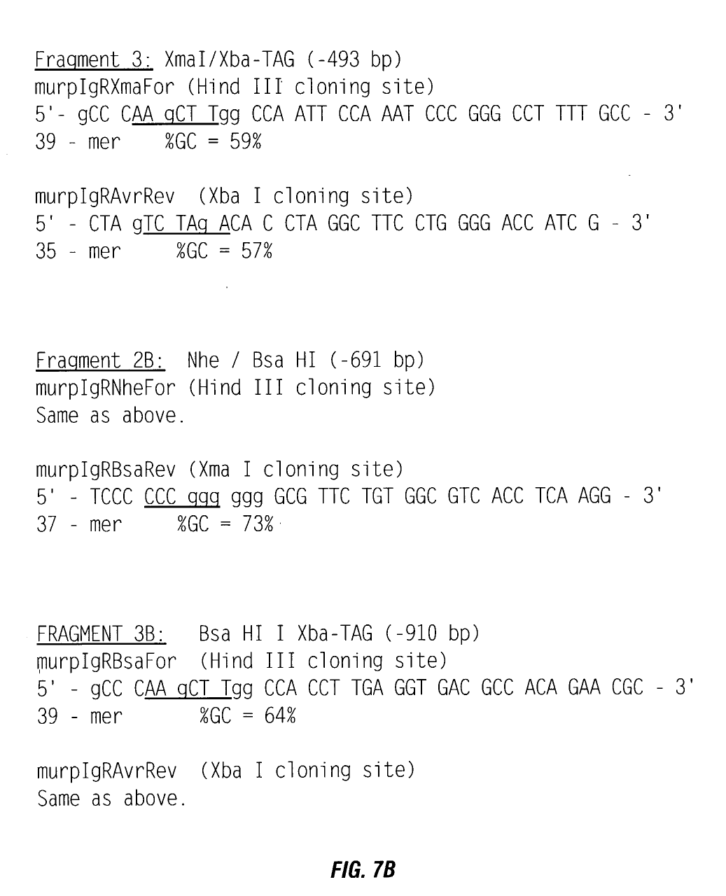

A mouse liver cDNA library (Clontech) was used as a source for template for the amplification of mouse pIgR sequences. As was the case for the rat pIgR cDNA's, the mouse cDNA was amplified as 5 separate fragments which can be combined to regenerate the entire mouse pIgR sequence (see FIG. 7). Alternatively, the sequences contained within separately cloned cDNA's may be used as a source for sequences that encode a mouse stalk molecule or sequences derived therefrom. As can be seen in FIG. 7, the primers used to amplify the mouse cDNA regenerated or introduced restriction enzyme sites into the cDNA for ease of subcloning and other subsequent manipulations. Each fragment was treated with the appropriate restriction enzymes and ligated into a cloning vector in order to generate an “intermediate vector”. The sequence of the cDNA in the intermediate vector was determined in order to confirm the sequence of the amplified DNA.

2.3. Cloning of Human pIgR cDNA

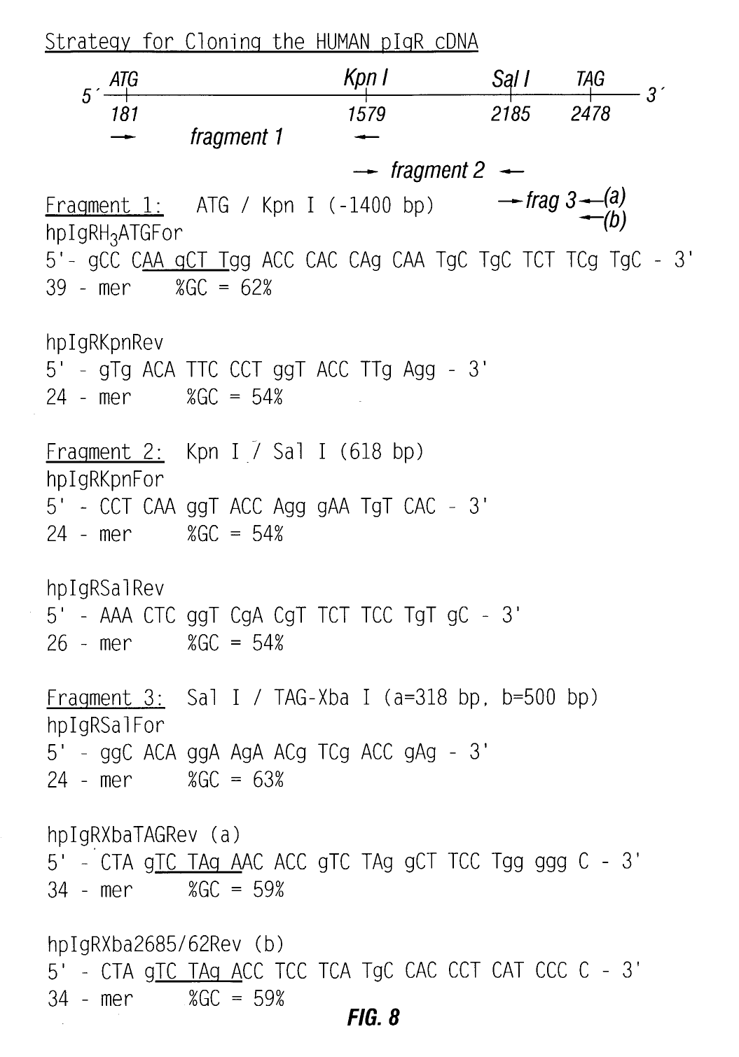

A human colon cDNA library (Clontech) was used as a source for template for the amplification of human pIgR sequences. The human cDNA sequences were amplified as 3 separate fragments which were inserted into intermediate vecors as described above (see FIG. 8).

2.4. Construction of Rabbit/Rat Chimeric pIgR

Expression of pIgR in Madin-Darby canine kidney (MDCK) cells using retroviral vectors has been described by Breitfeld et al. (Methods Cell Biol 32:329-37, 1989). The expression of rabbit and human pIgR in MDCK cells has been described, respectively, by Barroso et al. (J Cell Biol 1994 124:83-100) and Tamer et al. (J. Immunol 1995 155:707-14, 1995). Because rats are useful for in vivo assays, initial in vitro transcytosis assays used MDCK cell stransfected with rat pIgR. However, the expression of rat pIgR in transfected MDCK cells was reduced relative to results obtained with rabbit pIgR transfected MDCK cells. Without wishing to be bound by any particular theory, the relatively reduced expression of rat pIgR may be a consequence of an unusual structure in the 5′ untranslated region of the rat pIgR cDNA (Fabregat et al., Physiol Genomics 5:53-65, 2001; Fodor et al., DNA Cell Biol 16:215-25, 1997; Koch et al., Nucleic Acids Res 23:1098-112, 1995).

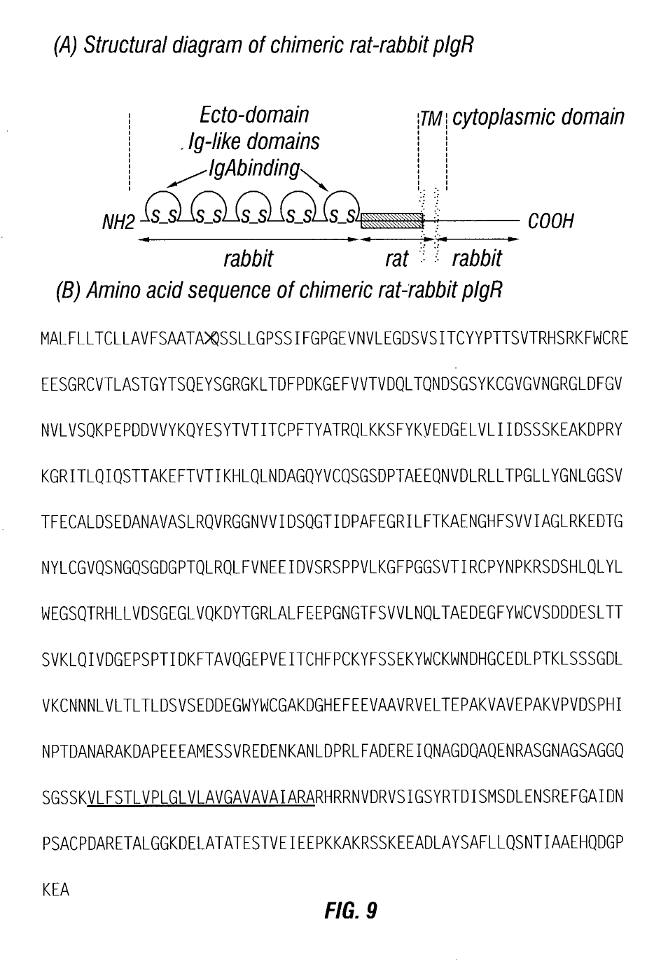

To enhance the production of a rat-like pIgR in transfected MDCK cells, a chimeric protein was produced via PCR using primers to rat and rabbit pIgR cDNA sequences and methods known in the art. The chimeric protein consists of amino acids 1-554 of rabbit pIgR, followed by amino acids 553-645 of rat pIgR, then amino acids 651-756 of rabbit pIgR. This chimeric protein contains the stalk and transmembrane regions of rat pIgR, and the remainder of the molecule is derived from rabbit pIgR. The chimeric pIgR has the same activity as wild type rabbit pIgR in pIgR assays such as transcytosis of IgA from the basolateral to the apical surface (forward transcytosis). The structure and amino acid sequence of the chimeric pIgR protein is shown in FIGS. 9A and 9B, respectively. The chimeric protein was expressed from an expression construct comprising the expression vector pCB7.

### Example 3

### GST-Stalk Fusion Proteins

3.1. GST-Stalk Fusion Proteins

GST-stalk fusion proteins are one type of pIgR target molecule. The GST (glutathionine-S-transferase, from Schistosoma japonica, unless otherwise indicated) polypeptide has several illustrative desirable attributes. It specifically binds glutathione, and with a sufficiently high affinity that it can be used to attach fusion proteins to solid surfaces coated with glutathione, and many such surfaces are commercially available; detectably labeled antibodies directed to GST epitopes are commercially available; and the GST amino acid sequences allow some fusion proteins to have enhanced attributes such as, e.g., enhanced solubility, biologically active conformations, and the like.

GST fusion proteins may optionally comprise elements useful for the detection, isolation, purification and manipulation of the GST fusion protein. Non-limiting examples of such elements include elements such as a 6×His tag, a FLAG tag, a c-myc epitope, a fluorescent polypeptide (e.g., GFP), a detectable enzymatic polypeptide (e.g. horse radish peroxidase, beta-galactosidase), or a biotin-binding polypeptide (e.g., avidin or streptavidin) polypeptide. GST fusion proteins are expressed in E. coli, purified on a glutathione column and attached to solid surfaces by known techniques (see, e.g., Smith et al., Unit 16.7, “Expression and Purification of Glutathione-S-Transferase Fusion Proteins” in Short Protocols in Molecular Biology, 2nd Ed., Ausubel et al., Editors, John Wiley & Sons, pp. 16-28 to 16-31, 1992).

Non-limiting examples of GST fusion proteins include those that comprise a portion of the stalk that contains the desired sites of reaction, e.g., domain 5 and domain 6, domain 6, or smaller portions of domain 6; or of any other regions of pIgR and stalk molecules such as those described herein in Tables 1 and 4. The fragment of pIgR or stalk molecule used in a GST fusion protein may change depending on the nature of a particular use of the GST fusion protein, but those skilled in the art will know what amino acid sequences are appropriate to include in a given GST fusion protein.

TABLE 10
GST-STALK FUSION PROTEINS

| | | Molecular | | Binds to |
|---|---|---|---|---|
| GST Fusion | | Weight of | 6xHis | single chain |
| Protein | Origin of Stalk | Fusion | Tag | antibody |
| Description | Sequences | Protein | present? | sFv5? |
| GST-Cyn | Cynomolgus | ˜37.6 kD | Yes | Yes |
| monkey-stalk | Monkey partial | | | |
| | cDNA | | | |
| GST-Human-stalk | Human cDNA | ˜37.8 kD | Yes | Yes |
| GST-Rat-stalk | Rat cDNA | ˜39.3 kD | No | Yes |
| GST-Rabbit-stalk | Rabbit cDNA | ˜38.8 kD | No | No |

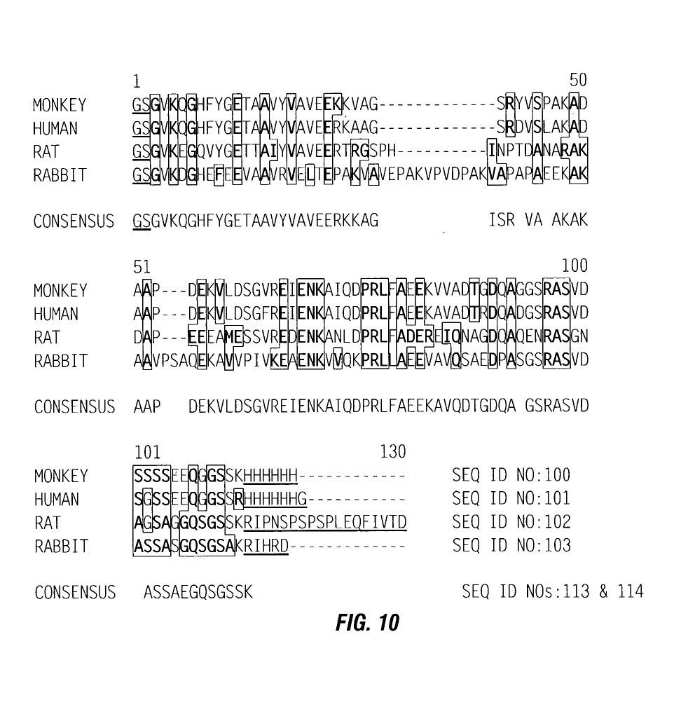

Table 10 summarizes the general characteristics of GST-stalk fusion proteins that are described in more detail in the subsequent subsections and in FIG. 10.

3.2. GST-(Cynmonkey Stalk) Fusion Protein

3.2.1. Prepartion of Cynomolgus Monkey (CynMonkey) Stalk cDNA Fragment

Rhesus and Cynomolgus monkey intestinal tissue was obtained from Yerkes Regional Primate Center (Atlanta, Ga.). At least 30 grams of tissue specimens were each prepared from ileum and colon sections where the tissue was excised within one-half hour postmortem, rinsed free of feces with PBS, and then rapidly frozen using liquid nitrogen, shipped overnight on dry ice and stored frozen at −80° C.

A section of cynomolgus colon weighing 5.3 grams (wet weight) was placed in a 50 ml conical tube and rapidly washed 3-5 times with approximately a 30 ml volume of PBS to remove residual fecal material. The colon segment was removed to a very small plastic weigh boat and a longitudinal incision was made exposing the luminal surface, which was quickly and gently rinsed with ˜50 mls of PBS. One (1) ml of TRIzol reagent (Life Technologies) was layered and massaged on the luminal surface, collected in a 15 ml conical tube, and total cellular RNA isolated as per manufacturer's instructions. Briefly, the RNA solution was centrifuged at 12,000×g to remove insoluble cellular debri, and 700 uls of total solution transferred to a microfuge tube. One hundred and forty (140) μls of chloroform was added to the solution centrifuged at 14,000 rpms for 15 minutes at 4° C. Four hundred and thirty (430) μls of aqueous phase was collected, 215 uls of isopropanol added, incubated at room temperature for 10 minutes, and the RNA precipitated by centrifugation at 14,000 rpms for 10 minutes at 4° C. The white pellet was washed with 1 ml of 75% ethanol, air dried for 5-10 minutes, and the RNA pellet resuspended in 50 uls of DEPC-treated water. Quantitation of total RNA was determined by spectrometry using the value of 1 OD₂₆₀ value+40 μg RNA/ml.

Synthetic degenerate DNA primers (prepared by Genset, Inc.) used in the first strand cDNA synthesis (RT-PCR) and PCR amplification of the cynomolgus monkey partial cDNA.

RT-PCR primer: EPKKAKRS-Low Reverse primer

| | |
|---|---|
| 5′- GTATCGATCTTTTTGCCTTCTTGGGYTC -3′ | (SEQ ID NO:_) |

PCR Forward primer: EKYWCKW Forward primer

| | |
|---|---|
| 5′- GGAATTCGARAARTAYTGGTGYAARTGG -3′ | (SEQ ID NO:_) |

PCR Reverse primer: EPKKAK-Low Reverse primer

| | |
|---|---|
| 5′- GTATCGATCXRTTXGCRTTRTTNGGRTC -3′ | (SEQ ID NO:_) |

Notes: “R” designates either an A or G purine base; “Y” designates either an C or T pyrimidine base; “N” designates either of the A, C, G or T bases; and “X” designates any base.

An oligonucleotide primer (SEQ. ID NO:______ RT-PCR primer) was used together with the SuperScript First Strand Synthesis Kit (Life Technologies, cat.#11904-018) to synthesize the first strand cDNA from 5 μg of total cynomolgus monkey RNA as per manufacturer's instructions. Briefly, 100 pmols of primer (SEQ. ID NO:______ RT-PCR primer) and 5 μg of total RNA was included in a 10 μl RT-PCR reaction, heated to 70° C. for 10 minutes, then cooled to 4° C. A 9 μl 10×RT-buffer mixture was then added to the RT-PCR reaction and incubated at 42° C. for 2 minutes, followed by the addition of 1 μl of SuperScript II enzyme to each reaction. The reverse transcription reaction allowed to proceed at 42° C. for 50 minutes. Proper control reactions were also assembled and run simultaneously. The reactions were terminated by heating to 70° C. for 15 minutes. To prevent interference of the RNA in the subsequent PCR amplification step, 1 μl of RNase H was added and the reaction incubated at 37° C. for 20 minutes before storing the single stranded cDNA material at −20° C.

A 2 μl aliquot of the cynomolgus monkey cDNA reaction was used in a 50 μl PCR reaction and a partial cynomolgous double stranded cDNA amplified using 0.2 uM concentration of the Forward (SEQ ID NO: ______, PCR Forward primer) and Reverse (SEQ ID NO: ______, PCR Reverse primer) primers together with 2.5 units of High Fidelity Platinum Taq (Life Technologies). Amplification was carried out as per manufacturer's instructions and thermocycling conditions as follows: (1) denaturation at 94° C. for 10 minutes; (2) 30 cycles of denaturation for 1 minute at 94° C., primer annealing for 1 minute at 60° C., primer extension for 30 seconds at 72° C., and (3) a final 4° C. storage step. The correct size of the 729 bp PCR product was confirmed by agarose gel electrophoresis. The entire PCR reaction was run on a preparative agarose gel and the 730 bp partial cDNA fragment separated from contaminating primers and purified using the Qiagen QIAquick purification kit. The purified partial cDNA fragment was re-amplified and purified as described above. Due to the utilization of Taq DNA polymerase, all PCR products will contain a 3′-A overhang and will be easily ligated into an intermediate vector using the TOPO TA Cloning Kit (Invitrogen, cat.#450640). The resulting PCR product was ligated into the pCR-II vector (Invitrogen) as per manufacturer's instructions and the ligation reactions transformed into TOPO One-shot competent cells (Invitrogen). Colonies were selected and 3 ml mini-cultures grown, miniprep DNA prepared using the Qiagen Miniprep Kit (Qiagen, cat.#27106), and positive clones identified by an Eco RI restriction enzyme analysis.

Eco RI digestion identified 4 positive clones containing the PCR DNA product. Maxiprep DNA was prepared (Qiagen DNA Maxikit) for two (2) clones and the DNA nucleotide sequence determined following sequencing of the DNA with both Sp6 (SEQ. ID# Sp6) and T7 (SEQ. ID# T7) sequencing primers (SDSU Microchemical Core Facility). The 730 nucleotide cDNA sequence encodes most of domain 5 through the cytoplasmic domain, which is homologous to a region of the human pIgR molecule corresponding to amino acids Glu474 through Ser717. Detailed sequence and alignment analysis comparing the human and cynomolgus monkey pIgR cDNAs demonstrate that the sequences differ in 18 amino acids within this 242 amino acid region (Glu474 through Ser717). The nucleotide and amino acid sequences for a simian pIgR are shown in FIG. 2.

3.2.2. Expression Construct Comprising the GST-(Cynmonkey Stalk) Fusion Protein

A plasmid comprising cynomolgus monkey pIgR sequences (“pTA-CynMonk-pIgR,” which is a derivative of PCR pCR-II plasmid, Invitrogen, having simian pIgR sequences) is used as a template for PCR amplification of the cynomolgus monkey pIgR stalk region using the CynMpIgRstalk-5′FOR and CynMpIgRstalk-3′REV sequencing primers. These primers allow for the use of a directional cloning strategy (BglII to EcoRI ligation) and result in the incorporation of a C-terminal 6×His tag that can be used to isolate or attach the fusion protein to a solid surface.

CynMpIgRstalkGST 5′FOR, a 5′-Forward PCR primer containing a BglII site (underlined) and having the sequence (SEQ ID NO:______):

5′-CGGGAAGATCTGGAGTGAAGCAGGGCCACTTCTATGG-3′

CynMpIgRstalkGST 3 ′REV, a 3′-Reverse PCR primer containing an in-frame 6×His tag and an Eco RI site (underlined) (SEQ ID NO:______):

5′-CGGAATTCCTAGTGATGGTGATGGTGATGTTTGGAGCTCCCACCTTGTTCCTCAGAGC-3′

The 309 bp PCR fragment is purified and subject to restriction digests using EcoRI and BglII enzymes, and the resulting 305 bp fragment is gel-purified. The purified EcoRI-BglII fragment is cloned into BamHI- and EcoRI-digested pGEX-2TK (Amersham Pharmacia), a plasmid that has a GST-encoding nucleic acid sequence that can be fused in-frame with a cloned DNA. The resulting plasmid is subjected to DNA sequence analysis to confirm the absence of any PCR-induced mutations and to verify that the GST and pIgR sequences are linked in-frame with each other. FIG. 10 shows the amino acid sequences of the GST-stalk fusion proteins.

3.3. Other GST-Stalk Fusion Proteins

GST fusion proteins derived from human, rat and rabbit stalk sequences were prepared essentially according to the methods and methods used in the preceding subsections the preparation of a GST-(Cynmonkey stalk) fusion protein. One exception is that the stalk sequences were, in some cases, amplified from the above-described cDNA intermediate vectors comprising fragments of the pIgR rat, rabbit and human sequences.

For example, in the case of the GST-(rabbit stalk) fusion protein, a plasmid comprising rabbit pIgR sequences (“pGST-RabplgRStalk”) was digested with BamHI and EcoRI, which liberates a 312 bp fragment. The 312 bp fragment was cloned into BamHI-EcoRI-treated pGEX-2TK vector, a plasmid that has a GST-encoding nucleic acid sequence that can be fused in-frame with a cloned DNA. The resulting plasmid was subjected to DNA sequence analysis to confirm the absence of any PCR-induced mutations and to verify that the GST and pIgR sequences are linked in-frame with each other.

### Example 4

### Preparation of Ligands Directed to Domain 6 and pIgR Stalk Molecules

4.1. Assays for Ligands An assay is prepared by applying purified pIgR stalk molecules or GST-pIgR stalk molecules, or any other pIgR target, to multiwell (48-well, 96-well and other size plates and allowing the protein to adhere to the wells of the plates during overnight incubation. The plates are washed to remove unbound proteins. Samples of the serum from the immunized mice are incubated with the pIgR or GST-pIgR coated plates. After 1 to 2 hours of incubation (gentle shaking at room temperature), the plate is washed free of unreacted immune serum proteins. Mouse antibodies that react with an immobilized GST-pIgR protein are detected by adding to each well a sample of a goat antibody that has been raised against and is directed to mouse immunoglobulin, i.e., all subclasses of murine immunoglobulins. The goat antibody is conjugated to an enzyme that is used for detection; non-limiting examples include horse radish peroxidase and alkaline phosphatase. After unreacted horse radish peroxidase or alkaline phosphatase conjugated goat anti-mouse immunoglobulin has been washed from the wells, the substrate of horse radish peroxidase or alkaline phosphatase is added. When the color is sufficiently developed, the reaction is stopped and quantitated using a spectrophotometer. In the positive wells, antibodies against the GST-pIgR protein will be present. Some of these antibodies are directed to the GST portion of the protein if GST-pIgR is used. By assaying against other GST fusion proteins, it is determined if the antibodies are against GST or pIgR. This assay is also used to identify antibody producing cells and clones in 96-well plates that are part of the process of isolating clones of hybridomas that produce the desired monoclonal antibody.

Beads that bind GST moieties on GST-fusion proteins are also used for assays. GST-pIgR bound to beads is reacted with sera that contain antibodies directed against pIgR. The antibodies that react with and bind to pIgR can then be detected by an anti-antibody conjugated to horse radish peroxidase or alkaline phosphatase. If the antibodies that react with pIgR are derived from mice, then the antibodies that detected the presence of the mouse antibody is obtained from another animal species, such as goat or sheep. Those skilled in the art will know how to adjust the source and specificity of the detecting antibody conjugates (i.e. horse radish peroxidase or alkaline phosphatase conjugated to anti-FLAG tag antibody) to obtain the desired results.

4.2. Preparation of Monoclonal Antibodies (Mabs)

Monoclonal antibodies are created by immunizing mice with portions of pIgR, generally prepared as oligopeptides having defined amino acid sequences. For example, a nucleic acid encoding an amino acid sequence found in a conserved region of pIgR, such as those described in Table 1, or an amino acid sequence that varies between homologs, such as, e.g., R1, R2a, R2b, R3a, R3b, R3c (etc.) (Table 4) is used to create a pIgR-target-GST fusion protein that is expressed in a host cell such as E. coli. The GST portion of the fusion protein is used to isolate the fusion protein, and the purified GST-pIgR protein is mixed with adjuvant and injected into mice to produce an immune response. The extent of the immune response is measured over time by removing blood from the immunized mice at regular intervals and measuring the level of antibodies directed to the GST-pIgR fusion protein using an immunoassay, e.g., an ELISA.

Once the immunized mouse has been shown to be producing antibodies directed to the GST-pIgR fusion protein, the spleen of the mouse is harvested, and cells therefrom are prepared for fusion with immortalized fusion partners, such as the NS/1 cell line, according to Kohler and Milstein, in order to create Mab-producing hybridoma cell lines. Independently isolated clones and subclones are grown to an appropriate density, the cell supernatant is assayed using an ELISA to determine if antibodies that react with the GST-pIgR fusion proteins are produced by each clone or subclone. Positive wells are assayed using limiting dilution, and clonal and subclonal cell lines are eventually obtained that produce Mabs against either the GST-pIgR fusion protein.

By assaying and comparing results from assays using commercially available monoclonal antibodies directed to GST, and GST fusion proteins that do not contain pIgR, as well as polyclonal antibodies directed to pIgR, it is possible to identify isolated Mabs that either are pIgR specific or are specific to an epitope not present in either pIgR or GST but which occurs at the junction thereof. The Mabs can additionally be tested for specificity using MDCK cells and MDCK cells that have been transfected with different species of pIgR (human, rat, mouse, pig, rabbit, monkey, etc.).

A collection of monoclonal antibodies and sFvs that cumulatively bind to many, preferably every, epitope of pIgR domain 6, which includes the pIgR stalk, is prepared. Each of the sFvs and the Mabs are epitope mapped using the nested set of overlapping oligopeptides (each comprising 5 to 20 amino acids). Linear epitopes and conformational epitopes are identified on the strength of their binding and the location of the peptides in the nested set.

4.3. Single Chain Antibodies

One type of pIgR-targeting element is an antibody, or an antibody derivative, directed to a transcytotic molecule such as the pIgR stalk. As a non-limiting example, single chain Fv antibody fragments (sFv) directed to epitopes in defined regions in the pIgR amino acid sequence may be used. Non-limiting examples of such sFv antibodies are shown in FIGS. 3 to 5.

A derivative of sFv5A that incorporates an epitope known as a “FLAG tag” is designated “sFv5AF” (FIG. 3). Due to the way in which it was constructed, the amino acid sequence of sFv5AF has a mutation relative to sFv5A that is denoted “Q5V” (Gln at position 5 changed to Val).

A derivative of sFv5AF that contains a cysteinyl residue near its carboxyl terminus is designated “sFv5AF-Cys” (FIG. 5). This derivative of sFv5AF has a cysteine residue at the carboxy terminal region was introduced into the reading frame encoding sFv5AF by PCR mutagenesis (see Example 5).

4.4. Calmodulin

Another source of amino acid sequences that provide ligands for pIgR is a protein known as calmodulin. There is evidence that calmodulin binds pIgR and it is thus expected that amino acid sequences within calmodulin interact with pIgR and may be isolated and used to prepare polypeptide ligands to pIR (Enrich et al., Hepatology 24:226-232; 1996; Chapin et al., J. Biol. Chem. 271:1335-1342; 1996).

4.5. pIgR-Targeting Elements Derived from Bacterial Proteins

Zhang et al. (Cell 102:827-837, 2000) have published studies that indicate that pIgR is exploited by bacteria to provide a mechanism by which bacterial cells have enhanced abilities to adhere, invade, and undergo apical to basolateral transmigration. These results provide pIgRtargeting elements that are derived from surface proteins of bacteria.

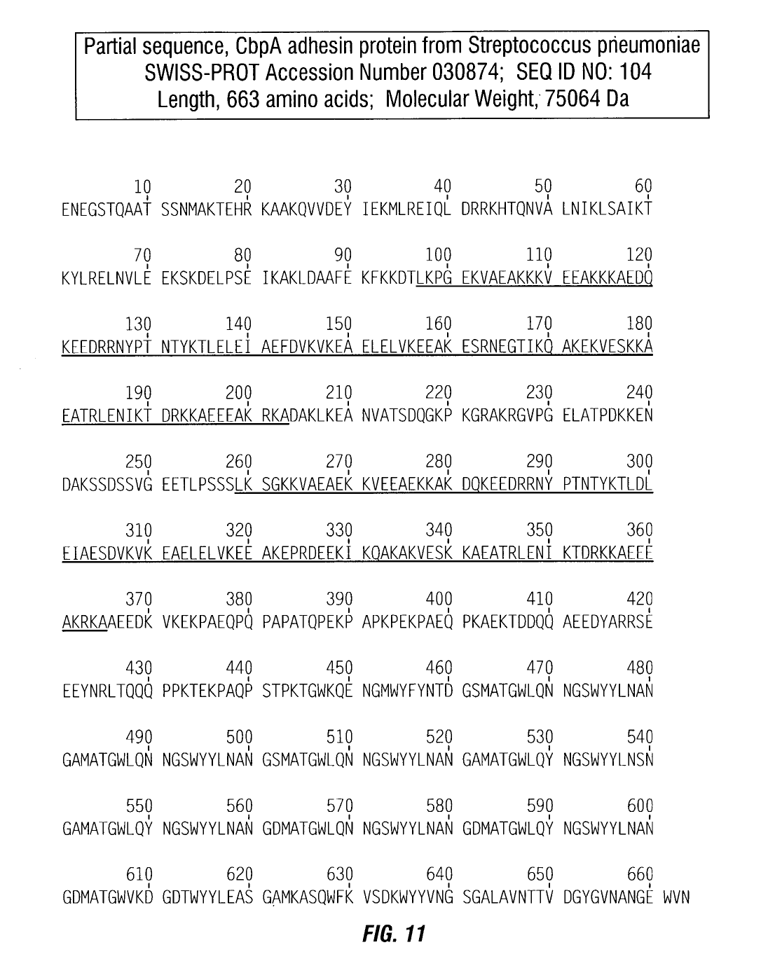

Zhang et al. present evidence that the pneumococcal adhesin protein CpbA interacts with human pIgR (hpIgR) as either a part of the outer surface of a bacterial cell or as a free molecule. The regions of CpbA:hpIgR interaction were mapped using a series of large peptide fragments derived from CpbA. CpbA (Swiss-Prot Accession No. 030874) contains a choline binding domain containing residues 454-663 and two N-terminal repetitive regions called R1 and R2 (SEQ ID NOS:______ and ______, respectively) that are contained in residues 97-203 and 259-365, respectively. Zhang et al. demonstrated that polypeptides containing R1 (107 amino acid residues) and R2 (see FIG. 11) interact with the SC portion of hpIgR, whereas a polypeptide containing residues 1-101 of CpbA does not bind to hpIgR.

Small polypeptides that retain the ability to interact with human and animal species of pIgR are utilized as pIgR targeting elements in the present invention. Such polypeptides may include those identified by phage display of disulfide constrained peptides as described above or polypeptides including but are not limited to the CbpA1, CbpA2, and CbpA3 polypeptides described by Zhang et al. In addition, other polypeptides from bacterial proteins homologous with CpbA, the pneumococcal adhesin protein in Streptococcus pneumoniae studied by Zhang et al., are part of the present invention. Such homologous proteins are present in virtually all pneumococcal serotypes. Those skilled in the art will be able to identify additional homologous proteins from genomic and protein databases such as Swiss-Prot, Entrez, and GenBank.

A search of Swiss-Prot revealed the following list of proteins (listed by Accession Number) that have sequences homologous with R1 and R2: O30874, O69188, O33741, O33742, Q9RQT5, AAF73779, AAF73781, AAF73788, AAF73814, AAF73790, Q9RQT3, Q9RQT2, AAF73776, AAF73786, AAF73792, AAF73798, AAF73807, AAF73810, AAF73812, AAF73822, AAF73795, Q9RQT6, AAF73785, Q9ZAY5, Q9RQT4, Q9RQT1, AAF73777, AAF73799, AAF73801, AAF73809, AAF73784, AAF73817, AAF73778, AAF73811, AAF73813; O33753, AAF73787, AAF73808, AAF73773, AAF73780, AAF73797, AAF73775, AAF73791, AAF73804, AAF73816, BAB01952, O58288, Q9Y102, and Q54972.

Smaller polypeptides comprising portions of the entire sequence of CbpA and proteins homologous to CbpA, and preferably portions of R1 and R2 and polypeptides homologous to R1 and R2, are identified based on their ability to bind to animal species of pIgR, preferably human pIgR. An overlapping, nested set of peptides can be synthesized and their ability to interact with pIgR can be tested to identify peptides that may be used to transport biologically active polypeptides, including vaccines, into (apical and basolateral endocytosis) and across (forward or reverse transcytosis) epithelial cell barriers. The peptides may be tested for their ability (i) to prevent SC binding to pIgR coated beads or (ii) to prevent adherence, invasion, or transmigration by S. pneumoniae R6x to Detroit cells, both methods being described by Zhang et al. The peptides may be from 5 to 100 amino acids long, preferably from 6 to 50, and most preferably from 6 to 20. An offset of 1 to 5 amino acids and preferably 3 to 4 amino acids may be used. A nested, overlapping set of peptides 15 amino acids long with an offset of 3 amino acids that would contain residues 1-15, 4-18, 7-21, 10-24, 13-27, etc., until the last residue in the polypeptide sequence is reached. By comparing the amino acids in peptides that are contiguous in CpbA and that show positive binding to pIgR, the core linear sequence that is required for binding to pIgR may be identified. A large peptide may be systematically reduced in size until the smallest peptide that produces a positive binding to pIgR is identified. Methods for identifying the core linear sequence have been described by Geysen et al. (J. Immunol. Methods 102:259-274, 1987), Tribbick et al. (J. Immunol. Methods 139:155-166, 1991), Geysen et al. (J. Molecular Recognition 1:32-41, 1988), Tainer et al. (Mol. Immunol. 23:709-715, 1986).

### Example 5

### Genetic Engineering of Single-Chain Antibodies

5.1. Introduction or Deletion of Reactive Groups

In vitro genetic manipulation has been used to alter the reading frame of sFv5A so as to create derivatives that have substitutions or insertions of amino acids with reactive sites. For example, sFv5AF-Cys is a derivative of sFv5AF into which a reactive Cys residue has been inserted, which also has one GGGGS linker between the newly introduced Cys residue and the sFv portion of the polypeptide (see FIGS. 3 to 5). The Cys residue contains a side group, —SH, that can react with the HS-side group of another Cys residue to form a disfulfide bond (—S—S—) that links the two Cys residues and the amino acids to which each Cys is attached. The positioning of a Cys residue in a sFv derivative influences whether it will react with a Cys residue in the same molecule (thus producing a monomer having an intramolecular disulfide bond) or a Cys residue in another sFv molecule (thus producing a multimeric sFv molecule having an intermolecular disulfide bond).

5.2. Introduction of Cysteine Residue into sFv5AF

The sFv single-chain molecule sFv5AF was altered via PCR mutagenesis in order to incorporate a cysteine residue at the carboxy terminal region. The template, a pSyn expression vector encoding sFv5AF, was amplified using a first oligonucleotide primer, “LMB3,” that has a sequence (5′-CAGGAAACAGCTAGAC-3′, SEQ ID NO:______) that is complementary to regions 5′ of the sFv5AF coding region in pSyn), and “cys-long,” a second oligonucleotide primer having the sequence:

(SEQ ID NO:_)

5′-AGTTGCGGCCGCGGCAGGAGCCACCGCCACCACCTAGGACGGTGAC

CTT

The latter primer is complementary to the last 4 codons of sFv5AF, with the 5′ end of the primer encoding the amino acid sequence GGGGSC in frame with sFv5AF, followed by a NotI restriction site.

Amplification was performed using the Taq-plus precision polymerase (Stratagene) according to the manufacturer's instructions. The PCR product was cleaved with NcoI and NotI, and then ligated into pSyn expression vector DNA that had been cleaved with NcoI and NotI. The resultant expression construct encodes sFv5AF-Cys, which has, from an amino- to carboxy-terminal direction, a pelb leader sequence (for secretion in E. coli) and a FLAG epitope tag, both encoded by vector sequences; sFv5AF-Cys, i.e., a heavy chain variable region, a spacer sequence [GGGGS repeated three times, i.e., (G4S)₃], a light chain variable region, another (G4S)₃ linker, a cysteine residue (emboldened “C”) that has been introduced into the sFv relative to sFv5AF; and c-myc epitope and 6×His tags encoded by vector sequences (see FIG. 4).

The amino acid sequence of any protein, including the single chain antibody sFv5A and its derivatives (sFv5AF, sFv5AF-Cys, etc.), is encoded by a nucleotide sequence, the reading frame. In vitro genetic manipulation is used to alter the amino acid sequence of sFv5A so as to favor the formation of dimers, trimers and other multimers; to add or enhance desirable attributes of sFv5A, and/or to reduce or remove undesirable attributes.

5.3. Introduction of Cysteine Residue into sFv5A

The sFv single-chain molecule sFv5A was altered via PCR amplification in order to substitute the myc-6×His-tags at the carboxy terminal region (FIG. 4) with a GGGG-Cys C-terminal tail. For this construct, PCR amplification reactions were assembled using High Fidelity Platinum Taq (Life Technologies) according to manufacturer's instructions (1×High Fidelity PCR buffer, 0.2 mM each dNTP, 2 mM MgSO4, 0.2 μM of each primer, 2.5 units Platinum Taq High Fidelity, and template DNA as required), which allows for “hot start” PCR to minimize the generation of early stage nonspecific priming events. Amplification was carried out using a modified procedure adapted from Roux and Hecker (PCR Cloning Protocols, B. A. White, eds., Humana Press, 1997, pp. 39-45), where thermocycling reactions were run using linked files in a PCR program as follows: 1) denaturation at 94° C. for 10 minutes; 2) 30 cycles of denaturation for 1 minute at 94° C., primer annealing for 1 minute at 60° C., primer extension for 60 seconds at 72° C., and 3) a final 4° C. chill step (until analyzed). The size of the PCR products were confirmed by agarose gel electrophoresis and then purified away from contaminating primers by spin column chromatography (Qiagen QIAquick purification kit).

The template, a pSyn expression vector encoding sFv5A, was amplified using a “Forward” oligonucleotide primer, “Pelb-5 Forward,” (SEQ ID NO:______) that is complementary to the 5′-portion of the pelb-coding sequence in the pSyn5A vector, and “pSynG4Cys Antisense,” a “Reverse” oligonucleotide primer with the sequence listed below.

| | |
|---|---|
| Pelb-5′Forward Primer. | |
| 5′- AAATACCTATTGCCTACGGCAGCC - 3′ | (SEQ ID NO:_) |
| pSynG4Cys Antisense Reverse Primer. | |
| 5′-CGGAATTCCTACTAGCAGCCACCGCCACCTGCGGCCGCTAGGACGGTGACCTTGGTCCC-3′ | (SEQ ID NO:_) |

The latter primer is complementary to 7 codons near the C-terminus of the sFv5A coding region, with the 5′ end of the primer encoding the NotI restriction site followed by the amino acid sequence GGGGC in frame with sFv5A, followed by a two (2) tandem TAG stop codons and an EcoRI restriction site.

The PCR product was cleaved with BamHI and EcoRI, and then ligated into pSyn expression vector DNA that had been cleaved with BamHI and EcoRI. The resultant expression construct encodes sFv5A-G₄Cys, which has, from an amino- to carboxy-terminal direction, a pelb leader sequence (for secretion in E. coli) encoded by vector sequences; sFv5A-Cys, i.e., a heavy chain variable region, a spacer sequence [GGGGS repeated three times, i.e., (G₄S)₃], a light chain variable region, another G₄S linker, and a C-terminal cysteine residue that has been introduced into the sFv relative to sFv5A, replacing the c-myc epitope and 6×His tags encoded by vector sequences shown in FIG. 4.

5.4. Introduction of a C-Terminal Cysteine Residue into sFv5A and sFv5AF

The sFv single-chain molecules sFv5A and sFv5AF were altered via PCR amplification in order to remove the NotI restriction site and substitute the myc-6×His-tags at the carboxy terminal region with a GGGG-Cys C-terminal tail. For this construct, PCR amplification reactions were assembled using High Fidelity Platinum Taq (Life Technologies) according to manufacturer's instructions as described above.

The template, a pSyn expression vector encoding sFv5A, was amplified using a “Forward” oligonucleotide primer, “Pelb-5 Forward,” (SEQ ID NO:______) that is complementary to the 5′-portion of the pelb-coding sequence in the pSynSA vector), and “5A-(deltaN)Gly4-Cys,” a “Reverse” oligonucleotide primer (SEQ ID NO:______) with the sequence listed below.

| | |
|---|---|
| Pelb-5′ Forward Primer. | |
| 5′- AAATACCTATTGCCTACGGCAGCC - 3′ | (SEQ ID NO:_) |
| 5A-(deltaN)Gly4-Cys Reverse Primer. | |
| 5′-CCGGAATTCGTCGACTCATCAGCAGCCTCCACCGCCACCTAGGACGGTGACCTTGGTCCC-3′ | (SEQ ID NO:_) |

The latter primer is complementary to the 7 C-terminal codons of the sFv5A coding region, followed by the amino acid sequence GGGGC in frame with sFv5A, followed by a two (2) tandem TAG stop codons and sequential SalI and EcoRI restriction sites.

The PCR product was cleaved with BamHI and EcoRI, and then ligated into either the pSyn-SA or pSyn-5AF expression vector DNA that had been cleaved with BamHI and EcoRI. The resultant expression constructs encode sFv5A-(deltaN)Gly₄-Cys or sFv5AF-(deltaN)Gly₄-Cys, respectively. The amino acid sequence contains, from the amino- to carboxy-terminal direction, a pelb leader sequence (for secretion in E. coli) plus/minus a FLAG epitope tag encoded by vector sequences; sFv5A-Cys, i.e., a heavy chain variable region, a spacer sequence [GGGGS repeated three times, i.e., (G₄S)₃], a light chain variable region, another G₄S linker, and a C-terminal cysteine residue that has been introduced into the sFv relative to sFv5A, replacing the NotI restriction site and the c-myc epitope and 6×His tags encoded by vector sequences shown in FIG. 4.

5.5. Length, Composition and Number of Linkers

The two variable regions of a sFv that combine to form a ligand binding site are known as V(H) and V(L). In a monomeric sFv, the V(H) and V(L) of each molecule are associated with each other. In one type of dimeric sFv, the V(H) of one monomer [V(H)1] is associated with the V(L) of another monomer [V(L)2], and vice versa [i.e., V(H)2 is associated with V(L)1].

The length and composition of the linker between the V(H) and V(L) regions in an sFv is one factor that influences the tendency of an sFv to form monomers or multimers (Todorovska et al., Design and application of diabodies, triabodies and tetrabodies for cancer targeting, J Immunol Methods Feb. 1, 2001;248(1-2):47-66; Arndt et al., “Factors Influencing the Dimer to Monomer Transition of an Antibody Single-Chain Fv Fragment”, American Chemical Society, Biochemistry 1993, 37, pp.12918-12926). For example, a sFv molecule in which there is a relatively short linker between the V(H) and V(L) regions may be less likely to fold back upon itself and form a monomer. Thus, “short linker” sFv derivatives are often more likely to form dimers, as their V(H) and V(L) regions must pair with, respectively, the V(L) and V(H) regions of a second sFv molecule. Often, sFv derivatives with relatively long linkers between the V(H) and V(L) regions may fold back upon themselves, and therefore may have a greater tendency to form monomers.

The number of linkers between the V(H) and V(L) regions of sFv5A has been altered to produce a set of sFv derivatives that is screened and assayed for desirable attributes. That is, the sFv5A derivatives are assayed for their ability to form either monomers or multimers, and multimeric forms are analyzed to determine whether dimers, trimers, and the like, or mixtures thereof, are present. Assays, including by way of non-limiting example those described herein, are performed on the derivatives in order to determine their paracellular transporting and transcytotic properties, pharmacokinetics, stability and the like, in absolute terms as well as compared to the unaltered sFv5A molecule.

Various amino acid sequences are known that may serve as suitable spacers in the compounds of the invention (for a review, see Simons, Spacers, probability, and yields, Bioconjug Chem 1999 Jan-Feb;10(1):3-8). Some non-limiting examples sequences that have been used in sFv's include include EGKSSGSGSESKEF, one or more copies of GGGGS [a.k.a. (G₄S)ₓ] (Newton et al., Angiogenin single-chain immunofusions: influence of peptide linkers and spacers between fusion protein domains, Biochemistry Jan. 16, 1996;35(2):545-53), GSGS [a.k.a. (GSGS)ₓ] and GSSG [a.k.a. (GSSG)ₓ].

Derivatives of sFv5 with varying V(H) to V(L) distances, the distance varying with the number of times the linker sequence GGGGS is present, have been prepared using an overlapping PCR technique in which the heavy and light chains [V(H) and V(L), respectively] are generated separately by PCR amplification. The V(H) and V(L) PCR products are engineered to contain overlapping complimentary sequences at their 3′ and 5′ ends, respectively. The PCR products are mixed, heated to 95° C. to melt the DNA, then cooled to 58° C., resulting in the annealing of the two (2) products through their complimentary overlapping linker (alternate length) sequences. The annealed and connected PCR products now serve as a full-length heavy-light chain DNA template for a second round of PCR amplification using a primer set complementary to the 5′- and 3′-sequences of V(H) and V(L), respectively. PCR amplification results in a full-length sFv which has an altered linker length incorporated between the heavy and light chains.

The parent sFv was either pSyn5A, pSyn-5AF or pSyn-5AF-Cys (FIGS. 3 and 4). sFv5AF and sFv5AF-Cys contain three repeat linkers, i.e., (GGGGS)₃ between their V(H) and V(L). Derivatives with one linker (GGGGS), four linkers, i.e., (GGGGS)₄ and five linkers, i.e. (GGGGS)₅ have been prepared, and derivatives with 2 linkers can be prepared in like fashion.

In order to generate heavy chain regions with varied C-terminal linker sequences, templates (the pSyn expression vectors encoding sFv5A or sFv5AF) were amplified using a “Forward” oligonucleotide primer, “Pelb-5 Forward,” (SEQ ID NO:______ that is complementary to the 5′-portion of the pelb-coding sequence in both the pSyn5A or pSyn5AF vectors, and a “Reverse” oligonucleotide primer corresponding to the 8 C-terminal codons of the 5A heavy chain variable sequence in-frame with either the GGGGS (SEQ ID NO:______), (GGGGS)₄ (SEQ ID NO:______) or (GGGGS)₅ (SEQ ID NO:______) linker sequence as listed below.

The following Oligonucleotides were used for generating heavy chain regions with varied C-terminal linker lengths.

| | |
|---|---|
| Pelb-5′ Forward Primer. | |
| 5′- AAATACCTATTGCCTACGGCAGCC - 3′ | (SEQ ID NO_) |
| Single GGGGS linker: 5A-GS-1 Reverse Primer | |
| 5′- TGACCCTCCGCCACCTGAGGAGACGGTGACCAGGGTGCC - 3′ | (SEQ ID NO ) |
| (GGGGS)4 linker: 5A AGS4-52/G Linker Reverse Primer | |
| 5′- GGACCCTCCGCCTCCTGAGGAGACGGTGACCAGGGTGCCACGGCC - 3′ | (SEQ ID NO:_) |
| (GGGGS)5 linker: 5A AGS5-S2/G Linker Reverse Primer | |
| 5′-GCTCCCTCCGCCTCCGGACCCTCCGCCTCCTGAGGAGACGGTGACCAGGGTGCCACGGCC-3′ | (SEQ ID NO: _) |

In order to generate light chain regions with varied N-terminal linker sequences, template, the pSyn expression vector encoding sFv5A, was amplified using a “Reverse” oligonucleotide primer, “5A-Sal-H6-Sal, Xho, Eco Reverse Primer” (SEQ ID NO: ______) that is complementary to the 8 C-terminal codons of the 5A light chain variable sequence in the pSyn5A vector, and is in-frame with sequences coding for NotI and SalI restriction sites, a 6×His epitope tag, a SalI site, tandem TAG stop codons, and XhoI and EcoRI restriction sites. This reverse primer was used with one of the three (3) “Forward” oligonucleotide primers corresponding to either the GGGGS (SEQ ID NO:______), (GGGGS)₄ (SEQ ID NO:______) or (GGGGS)₅ (SEQ ID NO:______) linker sequence in-frame with the 8 N-terminal codons of the 5A light chain variable sequence as listed below.

The following Oligonucleotides were used for generating light chain regions with varied N-terminal linker lengths:

| | |
|---|---|
| 5A-Sal-H6-Sal,Xlio,Eco Reverse Primer | |
| 5′-CGGAATTCCTCCAGCTACTAGTCGACCTAGTGATGGTGGTGATGGTGGTCGACTG | (SEQ ID NO:_) |
| CGGCCGCACCTAGGACGGTGACCTTGGTCCC-3′ | |
| Single GGGGS linker: 5A-GS-1 Forward Primer | |
| 5′- GGTGGCGGAGGGTCATCTGAGCTGACTCAGGACCCTGCT- 3′ | (SEQ ID NO:_) |
| (GGGGS)4 linker: AGS-4 5A-Linker Forward Primer | |
| 5′- GGAGGCGGAGGGTCCGGTGGAGGCGGTTCAGCCGGAGGTGGCTCTGGCGGTGGC | (SEQ ID NO:_) |
| GGATCGTCTGAGCTGACTCAGGACCC - 3′ | |
| (GGGGS)5 linker: AGS-5 5A-Linker Forward Primer | |
| 5′- GGAGGCGGAGGGTCCGGAGGCGGAGGGAGCGGTGGAGGCGGTTCAGGCGGAGG | (SEQ ID NO:_) |
| TGGCTCTGGCGGTGGCGGATCGTCTGAGCTGACTCAGGACCC - 3′ | |

To generate the heavy and light chains described above, PCR amplification reactions were assembled using the proofreading ProofStart DNA polymerase (Qiagen) according to manufacturer's instructions (1×ProofStart PCR buffer, 0.3 mM each DNTP, 0.1 μM of each primer, 2.5 units ProofStart DNA polymerase, and template DNA as required), which allows for “hot start” PCR to minimize the generation of early stage nonspecific priming events. Amplification was carried out using a modified procedure adapted from Roux and Hecker (PCR Cloning Protocols, B. A. White, eds., Humana Press, 1997, pp. 39-45) as described above. The sizes of the PCR products were confirmed by agarose gel electrophoresis and then purified away from contaminating primers by spin column chromatography (Qiagen QIAquick purification kit).

Following purification of the individual heavy and light chain PCR products, 50 ng of each corresponding fragment was mixed and subjected to a second round of PCR amplification using the Pelb-5′ Forward (SEQ ID NO:______) and the 5A-Sal-H6-Sal, Xho, Eco Reverse (SEQ ID NO:______) primers, and the ProofStart DNA polymerase in a 50 μl reaction as described above, except that the annealing step was carried out at 58° C. The full-length PCR products comprising the heavy and light chain variable regions separated by either a single GGGGS linker, or a (GGGGS)₄ or (GGGGS)₅ linker, were gel purified and digested with either NcoI and BamHI, or NcoI and EcoRI.

The 2^{nd}-step full-length PCR products containing either the single GGGGS linker, or the (GGGGS)₄ and (GGGGS)₅ linker versions cleaved with NcoI and BamHI, were then ligated into any derivatized sFv5A expression vector DNA (such as pSyn-5A, pSyn-5AF, sFv5AF-Cys, sFv5AF-G₄Cys, sFv5A-(deltaN)Gly₄-Cys, sFv5AF-(deltaN)Gly₄-Cys) that had been cleaved with NcoI and BamHI. The resultant expression construct encodes an sFv5A derivative which has incorporated either the single GGGGS, (GGGGS)₄ or (GGGGS)₅ alternative linker between the heavy and light chain variable regions and maintains the integrity of the C-terminal amino acids. For various linker versions cut with Nco 1 and EcoRI, the resulting expression constructs will have alternative linkers between the heavy and light chain variable regions and a C-terminal 6×His epitope tag. The amino acid sequence contains, from the amino- to carboxy-terminal direction, a pelb leader sequence (for secretion in E. coli) plus/minus a FLAG epitope tag encoded by vector sequences; sFv5A-Cys, i.e., a heavy chain variable region, a linker region of either GGGGS, (GGGGS)₄ or (GGGGS)₅ codons, a light chain variable region; and either a C-terminal Cys or a single 6×His epitope tag at C-terminus.

The sFv5A and sFv5AF derivatives are expressed in E. coli cells and prepared from the periplasmic space of the bacterial cells using the same techniques and materials as those used for sFv5AF. Monomers and, if present, dimers and other multimers, are prepared and separated as described in the Examples and throughout the disclosure.

Similarly, derivatives with from 5 to 30 linkers are prepared. Other sFv5A derivatives may have varying numbers of other linkers. Any number or type of linker may be incorporated into an sFv derivative that is produced and tested for desirable properties. The spacing between the sFv sequences (the combined sequences of V(H) and V(L)) and other elements is altered for various properties. For example, it may be desirable to alter the positioning of polypeptide purification or detectable elements, or reactive groups, further from or closer to the sFv portion of the fusion protein depending on a particular purification strategy or intended use.

### Example 6

### Purification and Evaluation of Monomeric and Dimeric sFv5AF-Cys Molecules

6.1. Reduction of sFv5AF-Cys

A preparation of monomeric sFv5AF-Cys was reduced by adding dithiothreitol (DTT) to a final concentration of 10 mM, and incubating at 25° C. for 30 minutes. DTT provides quantitative reduction of disulfide bonds in proteins when used at about at least 20-fold excess. As DDT readily maintain monothiols completely in the reduced state and can quantitatively reduce disulfide bridges, treatment with DTT disfavors the formation of sFv5AF-Cys dimers wherein the monomers are covalently linked by a disulfide bridge, and thus favors the formation of sFv5AF-Cys monomers.

6.2. Size Exclusion Chromatography

Monomeric sFv5AF-Cys molecules were separated from dimers (and higher multimers if any are present) by size exclusion chromatography (SEC) on a 1×44 cm Superdex 75 column with 0.1 M PO₄ containing 1 mM EDTA, pH 6.25. Fractions 29-34 were collected as dimer, and fractions 38-43 were collected as monomer.

6.3. Transcytosis Assay Design

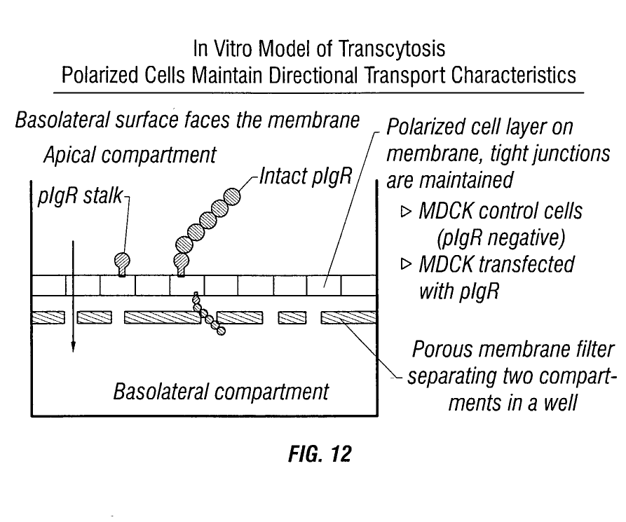

The assay was performed in the transwell system as shown in FIG. 12. The cells are grown on a porous membrane that separates the apical and the basolateral compartment. Complexes and compounds to be tested are placed in the apical compartment and then assayed by removing samples from the basolateral compartment. The direction of normal IgA transport is from basolateral to apical; however, preferred complexes and compounds of the invention undergo “reverse” (apical to the basolateral) transcytosis.

The complex or compound is placed in the apical compartment of the transwell and, after a period of time, a sample from the apical compartment and a sample from the basolateral compartment are removed and separated by SDS-PAGE. After gel electrophoresis, the proteins are transferred to PVDF membranes which are probed as Western blots with a polyclonal anti-sFv antibody, or an antibody to an epitope in the complex or compound being tested, followed by anti-rabbit IgG-alkaline phosphatase detection with nitro blue tetrazolium and 5-bromo-4-chloro-3-idoly phosphate, toludinium salt (NBT/BCIP). Western blotting with anti-sFv5A detects any molecular species that contains the sFv; regardless of whether the sFv is present either as part of a composition or compound of the invention or as a “free” sFv molecule.

Control (untransformed) MDCK cells do not contain significant levels or any pIgR. Therefore, one expects to observe no transcytosis of pIgR- or stalk-targeted complexes or compounds in these cells. Thus, a sample of the apical compartment will contain the complex or compound that has been added thereto, whereas a sample of the basolateral compartment should not show any sFv or conjugate thereof.

In contrast, MDCK cells that have been transformed with an expression construct that encodes and expresses a pIgR or stalk molecule, or derivatives thereof, have the capacity for pIgR-specific transcytosis. In these cells, one expects to observe transcytosis from the apical to basolateral compartments. Accordingly, bands corresponding to complexes or compounds will be present in the basolateral compartment if the molecules are capable of trancytosis.

Typically, four-day old cultures of MDCK cells expressing pIgR or stalk molecules (or derivatives thereof), or control (untransfected) MDCK cells, are incubated in the presence of 10 ng/ml to 10 mg/ml of the complex or compound to be tested in the apical chamber of a Transwell transcytosis chamber. The cells are incubated for at various times, typically about 20 hours unless otherwise indicated.

Both the apical and basal chambers are harvested at the end of the incubation period. Typically, one-third of the volume of media from the apical chamber, and all of the media from the basal chamber, are incubated with Protein A-Sepharose beads. Protein A binds to the Fc regions of IgG molecules, and binds to some sFv's through their VHIII domain. Although it binds sFv5 and its derivatives, Protein A does not bind to most sFv's. However, immunoprecipitation can be used as an alternative to Protein A. For example, the polyclonal antibody directed to sFv5 and its derivatives could be used to immunoprecipitated with sFv5 molecules.

The beads are washed, resuspended in SDS-PAGE sample buffer and the proteins subjected to SDS-PAGE. The proteins are transferred to PVDF and the membranes are probed as Western blots with a polyclonal anti-sFv antibody (or other antibody as appropriate, as described herein) followed by detected with, e.g., anti-rabbit IgG-alkaline phophatase and the calorimetric substrate NBT/BCIP.

In some experiments, the dimeric form of sFv5AF-Cys runs as doublet. This is likely due to the loss of a carboxy terminal polypeptide that comprises c-myc epitope and His tag amino acid sequences. When the sFv5AF-Cys dimer is probed with a monoclonal antibody directed to an epitope in the c-myc tag (9E10, Cambridge Bioscience), which is located on the amino terminus of the protein, the lower band of the doublet is not detected.

6.4. Transcytosis Assay

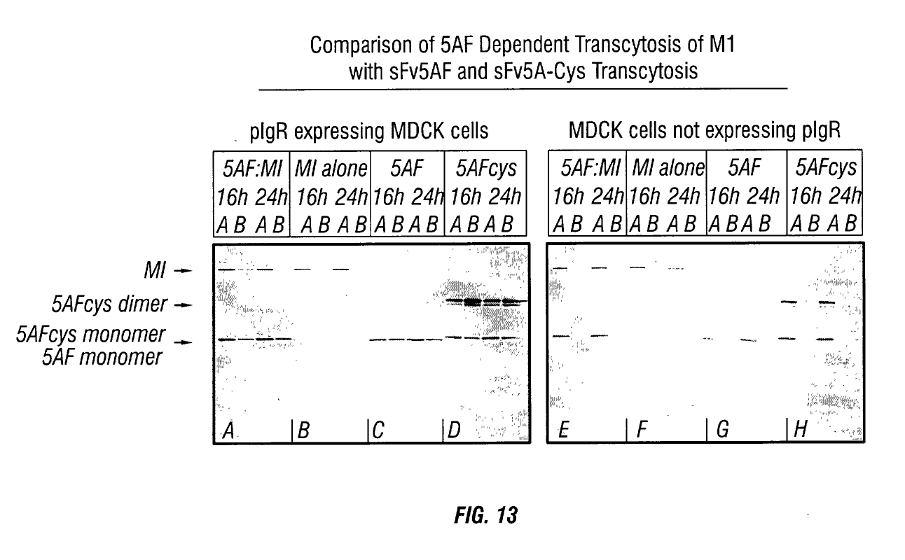

The transcytotic properties of sFv5AF (monomer) and sFv5AF-Cys (monomer and dimer) were evaluated and compared in MDCK cells. As shown in FIG. 13, sFv5AF efficiently transcytosed from the apical to basolateral media in a pIgR-dependent fashion. That is, reverse transcytosis of sFv5AF occurred in MDCK cells transfected with and expressing pIgR, but not in untransfected cells.

Transcytosis of a preparation of sFv5AF-Cys that contained both monomers and dimers was also evaluated. The results are shown in FIG. 13 (note that the sFvAF-Cys monomer has a slightly higher apparent molecular weight than the monomer of sFv5AF due to the relative addition of a Cys residue and a GGGGS linker). Transcytosis of the monomeric and dimeric forms of the sFv5AF-Cys molecule was pIgR-specific. Comparison of intensity of the dimer and monomer bands in the basolateral media in pIgR-expressing cells at 16 and 24 hours indicates that, compared to the monomer, more of the dimer has transcytosed at these time points.

6.5. Time Course of Transcytosis

A mixture of monomers and dimers of sFv5AF-Cys was assayed as described above in pIgR-expressing MDCK cells over defined periods of time. The periods chosen were 0-2, 2-4, 4-6, 6-8, 8-12 and 12-24 hours after the introduction of material to the apical chamber.

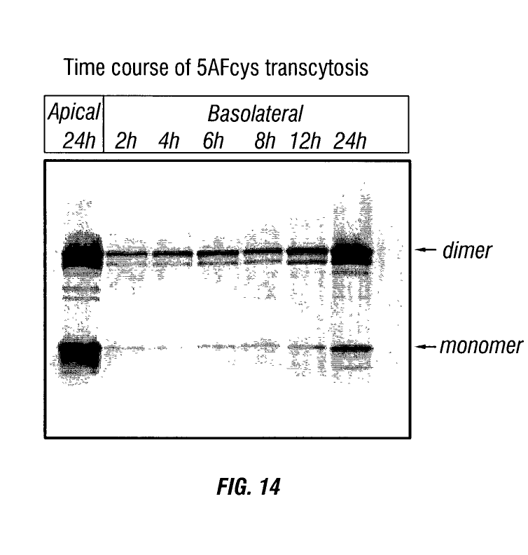

The results are shown in FIG. 14. The doublet band, which represents dimers, underwent apical to basolateral transcytosis at a faster rate than monomers. The transcytosis of both species is relatively constant over the time course.

6.6. Forward and Reverse Transcytosis Compared

The single-chain antibody sFv5AF-Cys was applied to either the apical compartment or the basolateral compartment of pIgR expressing or control MDCK cells at a concentration of 6 ug/ml. After 16 hours, apical and basolateral media were collected and 10% of the volume of the side to which ligand was added and 100% of the volume of the side representing transcytosed ligand were affinity-purified using Protein A sepharose and subjected to SDS-PAGE and Western blotting with anti-sFv5AF polyclonal antiserum. Thus, equal intensity bands in the apical and basolateral lanes represents 10% transcytosis.

The apical to basolateral (reverse) transcytosis of 5AF-Cys dimer was greater than 10%, while that of monomer was detectable, but less than 5%. In contrast, basolateral to apical (forward) transcytosis of 5AF-Cys dimer was less than 10%, and monomer transcytosis is undetectable.

### Example 7

### Transcytosis of Complexes Comprising Monomers or Dimers of sFv5 Molecules

M1 is a murine anti-FLAG-Tag Monoclonal Antibody that is commercially available (Sigma-Aldrich, St. Louis, Mo.). M1 may be used for detecting amino-terminal FLAG fusion proteins by immunoprecipitation, immunoblotting, or EIA, as it binds to the FLAG epitope when it is located at the free amino-terminus of a fusion protein. Typically, M1 does not bind to Met-FLAG fusion proteins, so will not recognize unprocessed, cytoplasmically expressed proteins.

M1 (0.4 ug per filter) was combined with 5AF (2 ug per filter) or buffer alone. After a 1 hour incubation at room temperature, transcytosis assays were carried out for 16 to 24 hours. Equilibrium appears to have been reached by 16 hours of transcytosis. Transcytosis of sFv5AF alone and sFv5AF-Cys was also assayed. Proteins were conentrated by affinity concentration 100% of the basolateral media, or 10% of the apical media. Samples containing sFv5AF or sFv5AF or sFv5AF-Cys were concentrated using protein A. Samples containing M1 were concentrated with protein A and protein G. Samples were analyzed by non-reducing SDS-PAGE and western blotting. M1 was used to probe sFv5AF and sFv5AF-Cys. Alkaline phosphatase-conjugated anti mouse IgG was then used as a probe to detect sFv5AF and sFv5AF-Cys, as well as the M1 from the transcytosis assay.

As is shown in FIG. 13, the sFv5AF (a mixture of ˜20% dimer and ˜80% monomer) causes or enhances the reverse transcytosis of M1 in a pIgR-dependent manner. No M1 is detected in the basolateral compartment when sFv5 is not present, whether pIgR is present or not.

The transcytosis of sFv5AF-Cys was also examined. A mixture of monomers and dimers of sFv5AF-Cys was used. Although transcytosis of both monomers and dimers was pIgR-dependent, the % of transcytosed dimers was greater than that of monomers present in the same sample.

### Example 8

### Transcytosis of Compounds Comprising Monomers or Dimers of sFv5A Molecules

Chemical conjugates of salmon calcitonin and sFv5AF-Cys monomers or dimers were prepared. These conjugates are examples of a biologically active molecule (calcitonin) covalently associated with monovalent (sFv5AF monomers) and multivalent (sFv5AF dimers) ligands directed to a molecule that confers transcytotic properties to complexes bound thereto.

8.1. Salmon Calcitonin Derivatization by Crosslinkers

Different cross-linkers were screened for the ability to derivatize salmon calcitonin (sCalcitonin) without forming a precipitate of the protein. It was found that addition of acetonitrile to the derivatization reaction helped prevent protein precipitation. A list of the cross-linkers screened and whether a precipitate formed is shown in Table 11, followed by diagrams showing the chemical structure of each cross-linker.

TABLE 11
CROSSLINKERS USED TO DERIVATIZE SALMON
CALCITONIN

| | | Preciptation With: | |
|---|---|---|---|
| Crosslinker | Chemical Nature of Crosslinker | Aqueous | w/ Acetonitrile |
| SPDP | NHS ester & pyridyldithio | Ppt | Ppt |
| Sulfo-LC-SPDP | NHS ester & pyridyldithio | Ppt | Ppt |
| SMPT | NHS ester & pyridyldithio | Ppt | Ppt |
| Sulfo-LC-SMPT | NHS ester & pyridyldithio | Ppt | Ppt |
| LC-SMCC | NHS ester & maleimide | Ppt | Ppt |
| Mal-Sac-HNSA | HNSA & maleimide (noncleavable, H₂O sol.) | No ppt | No ppt |
| SATP | Thiolation reagent (protected) | Ppt | Ppt |

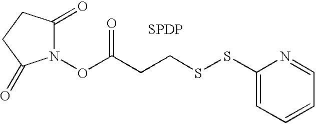

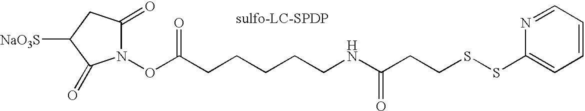

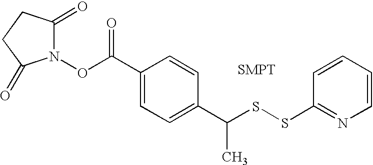

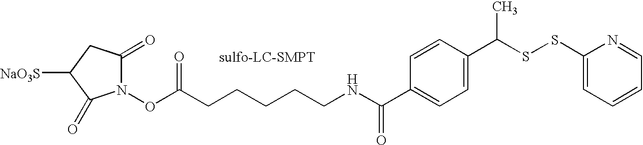

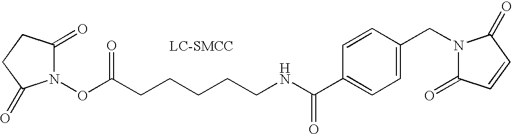

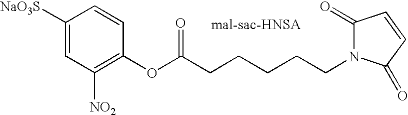

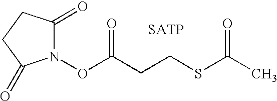

8.2. Conjugation of Salmon Calcitonin to sFv5AF-Cys and Purification of Monomeric and Dimeric Conjugates

8.2.1. sFv5AF-Cys-malsac-sCalcitonin Conjugation Reaction

On the basis of the cross-linker screening, mal-sac-HNSA was chosen as the cross-linker to be used in the sFv-calcitonin conjugation.

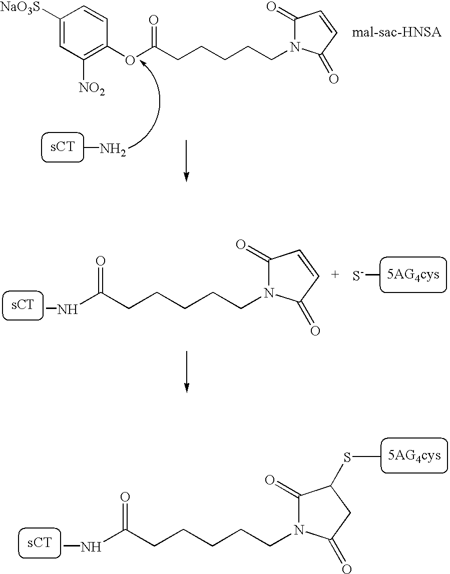

5AG₄-Cys monomer was purified by size exclusion chromatography (SEC). sFv5AG4-Cys was reduced and the monomer and dimer fractions were separated by SEC on a 1×44 cm Superdex 75 column in 10 mM PO₄ containing 100 mM NaCl and 1 mM EDTA, pH 6.25.

8.2.2. sFv5AG4-Cys-malsac-sCalcitonin Conjugation

Salmon calcitonin (sCT) was derivatized with a 5-fold molar excess of mal-sac-HNSA until a calculated substitution ratio of 0.94 was reached, as observed by monitoring OD412. The excess mal-sac-HNSA was removed by desalting on a 5 ml G25 column (Pharmacia HiTrap). The derivatized sCT was added to the 5AG₄Cys monomer and the reaction was incubated at 25° C. for 3 hours.

8.2.3. Purification of the sFv5AG₄-Cys-malsac-sCalcitonin Conjugates

The sFv5AG₄-Cys-malsac-sCalcitonin conjugate was purified by SEC using a 1×44 cm Superdex 75 column in 0.1 M PO₄, pH 7.5, containing 1 mM EDTA. Peak fractions were collected and analyzed by Coomassie-stained SDS-PAGE. A significant amount of the monomer sFv5AG₄-Cys-sCT conjugate had dimerized, and the dimer conjugate was collected included in the transcytosis assay as well as the monomer sFv5AG₄-Cys-sCT conjugate.

8.3. Conjugation of Monomer and Dimer sFv5AF-Cys to Salmon Calcitonin Using Mal-Sac-HNSA

8.3.1. Preparation of sFv5AF-Cys

9.5 ml (10 mg/ml, 0.36 mM) of sFv5AF-Cys was incubated with 10 mM DTT for 30 minutes and then was purified using size exclusion chromatography (SEC) on a 1.6×60 cm Superdex 75™ column in 100 mM PO₄, 100 mM NaCl and 1 mM EDTA, pH 6.25. Three sequential runs were performed to purify monomer and dimer sFv5AF-Cys. The sFv5AF-Cys was resolved into monomer and dimer sFv5AF-Cys peaks. Dimer and monomer sFv5AF-Cys were quantitated after pooling by measuring A₂₈₀ and the protein yield of dimer sFv5AF-Cys was calculated to be 26.4 mg with a concentration of 1.2 mg/ml or 0.043 mM. Monomer sFv5AF-Cys was calculated to have a protein yield of 30 mg with a concentration of 1.18 mg/ml or 0.042 mM.

8.3.2. Derivatization of Salmon Calcitonin (sCT)

Salmon calcitonin was desalted on a 24 ml P2™ column in 30% acetonitrile, 100 mM PO₄, 1 mM EDTA, pH 7.25. The concentration of sCT was calculated to be 22.3 mg/ml by measuring A₂₈₀. Since sCT is known to be soluble to at least 9.0 mg/ml, the 22.3 mg/ml sCT was diluted down to 9.0 mg/ml to prevent precipitation from occurring. 5.5 ml (9.0 mg/ml) sCT was used for the monomer conjugation and another 5.5 ml (9.0 mg/ml) sCT was used for the dimer conjugation. The 5.5 ml (9.0 mg/ml) sCT for the monomer conjugation was derivatized with a 5-fold molar excess of mal-sac-HNSA. The reaction was incubated at 25° C. for an hour until a 1:1 substitution ratio was reached by monitoring A₄₀₆. The mal-sac-sCT reaction was desalted immediately on the 24 ml P2™ column in 100 mM PO₄, 1 mM EDTA, pH 7.25. The derivatization was repeated for the 5.5 ml (9.0 mg/ml) dimer conjugation. By measuring A₂₈₀, the protein yield of both the monomer and dimer mal-sac-sCT derivatization was calculated. The mal-sac-sCT for the monomer conjugation was calculated to have a protein yield of 14.9 mg (2.7 mg/ml, 0.79 mM). The mal-sac-sCT for the dimer conjugation was calculated to be 22 mg (3.7 mg/ml, 1.08 mM).

8.3.3. Conjugation of Monomer and Dimer sFv5AF-Cys to mal-sac-sCT

An 8-fold molar excess (14.9 mg) of mal-sac-sCT, for the monomer conjugation, was added to 14.9 mg monomer sFv5AF-Cys with a final volume of 18 ml. An 8-fold molar excess (22.0 mg) of mal-sac-sCT, for the dimer conjugation, was added to 22.0 mg dimer sFv5AF-Cys with a final volume of 24 ml. The reactions for both monomer and dimer conjugation were incubated at 4° C. overnight. After the incubation, both of the reactions were concentrated down to 3 ml using the Pall Gelman 10K Centricon™. The monomer and dimer conjugates were purified on the 1.6×60 cm SEC Superdex 75™ column in PBS.

8.4. Comparison of Transcytosis of sCT-[Monomeric sFv5AF-Cys] Conjugates and sCT-[Dimeric sFv5AF-Cys] Conjugates

8.4.1. Procedure

Duplicate assays were carried out in which 1 μg of sFv or conjugate molecules was added to the apical chamber of MDCK cells expressing chimeric pIgR or control MDCK cells. Transcytosis was carried out for 11 hours at 37 degrees. Apical and basolateral media were collected, volumes adjusted to 1 ml, and 500 μl of the basolateral media and 50 μl of the apical media were subjected to protein A sepharose precipitation. Samples were analyzed by non-reducing SDS-PAGE and Western blotting with an anti-5A antibody.

8.4.2. Results

8.4.2.1. SDS-PAGE Analysis

Both sFv5AG₄-Cys and conjugates thereof show bands corresponding to monomeric and dimeric forms on non-reducing SDS-PAGE (“gel-monomer” and “gel-dimer”, respectively). Most of the gel-dimer molecules are probably disulfide linked. In the case of sFv5AG₄-Cys, a disulfide bridge could be formed between the engineered C-terminal cysteines and/or between internal cysteines. The latter event might occur during boiling of the samples in SDS prior to sample loading.

In the case of the conjugates, since one of the c-terminal cysteines is linked via a thioether bond to calcitonin, forms migrating as dimer on SDS-PAGE are probably due to disulfide bridges forming between internal cysteines in the sFv during boiling in SDS, or between a C-terminal cysteine on one sFv molecule and an internal cysteine on another sFv molecule. About half of the dimer conjugate preparation migrates as dimer, probably reflecting the efficiency of cross-linking of the dimeric molecules during boiling. In the monomer conjugate preparation, a small fraction of the conjugate migrates as dimer. The dimeric material is enriched in the basolateral media.

8.4.2.2. sFv5AG₄-Cys

The sFv5AG₄-Cys preparation is a mixture of monomers and dimers. A substantial portion of the sFv preparation migrates as a dimer on non-reducing SDS-PAGE. The dimer species was probably produced by covalent or non-covalent interactions that occurred prior to boiling in SDS. Thus, by comparing the monomer and dimer bands on the gel, one can monitor monomer and dimer transcytosis in the same sample. Transcytosis of sFv5AG₄-Cys dimers is typically greater than 10%, whereas transcytosis sFv5AG₄-Cys monomers is usually less than 10%, often less than 5%.

8.4.2.3. sFv5AG₄-Cys-Calcitonin Conjugates

The preparation of monomer sFv5AG₄-Cys-calcitonin that was tested shows 2 conjugate species on SDS-PAGE. These species of conjugates behave differently. Transcytosis of the gel-monomer conjugate is relatively inefficient, resembling that of the sFv5AG₄-Cys monomer. In contrast, transcytosis of the gel-dimer conjugate is relatively efficient.

Taken together, these results suggest that for sFv5AG₄-Cys, and for sFv5AG₄-Cys-calcitonin conjugates, dimeric forms of sFv5AG₄-Cys show more efficient transcytosis than the corresponding monomeric forms.

### Example 9

### Evaluation of Binding Characteristics via Surface Plasmon Resonance

9.1. Experimental Procedure

Each sFv or sFv-conjugate was tested for its ability to bind recombinant pIgR Domain 6 GST (D6-GST) fusion proteins. The pIgR Domain 6 fusion proteins were constructed from human, cynomologous monkey and rat cDNA sequences (see Example 3) and expressed in E. coli. A BIAcore® biosensor (Pharmacia Biosensor AB, Uppsala, Sweden and Piscataway, N.J.) was used to measure 5AF antibody fusion or antibody conjugate specific binding in real time to domain 6 in a capture format. The Biacore analysis was performed using a capture protocol in which an anti-GST antibody was immobilized to the Biacore chip surface, and a particular D6-GST fusion protein was bound to the antibody-coated surface. The sFv5AF or sFv5AF-containing conjugate was assayed typically over a concentration range of 15.6 to 500 nM.

BIAcore® kinetic evaluation software (BIAevaluation version 3.1) was used to determine the off association on rate (ka), dissociation off rate (kd) as well as the affinity constant (K_{A}=ka/kd or K_{D}=kd/ka). Binding was quantified by global fitting the data for the antibody concentration range using a 1:1 Langmuir binding model.

9.2. Comparison of sFv5AF-Cys Binding to Purified Monomer or Dimer sFv5AF-Cys Binding

There is little variation in the K_{D} among species as reflected in Table 13. When comparing the binding of sFv forms to one particular species of D6-GST fusion, the purified dimer exhibits a higher affinity than the purified monomer. The difference is ˜3-fold for each species (human, rat, simian). The sFvAF-Cys starting material, which is a mixture of monomer and dimer, has an affinity that is similar to the purified dimer. This may be due to the single binding site modeling of the data when the sFv5AF-Cys starting material is a mixture of monomer and dimer forms of sFv5AF-Cys. When comparing the purified monomer and purified dimer forms of sFv5AF-Cys, the differences in K_{D} appear to be due to changes in the association rate constant (ka). The dissociation rate constant (kd) does not vary between the monomer and dimer forms of sFv5AF-Cys.

In sum, the affinity of the sFv for the receptor is ˜3-fold higher for the purified dimer sFv compared to the purified monomer sFv; the differences are mostly due to differences in ka; and there is no significant variation in binding to D6-GST fusions from the different species tested.

TABLE 12
COMPARISON OF 5AF-CYS BINDING TO PURIFIED MONOMER OR
DIMER 5AF-CYS BINDING
1:1 binding model

| | Mono/ | | | | | | | | | MW |
|---|---|---|---|---|---|---|---|---|---|---|
| Analyte | Dimer | Ligand | ka (1/Ms) | kd (1/s) | Rmax | Conc | KA (1/M) | KD (M) | chi\hat{ }2 | Analyte |
| 5Afcys | | Human | 9.76E+05 | 1.45E−03 | 96.5 | global fit* | 6.71E+08 | 1.49E−09 | 2.20E+01 | 28884 |
| 5Afcys | Monomer | Human | 4.28E+05 | 2.41E−03 | 85.4 | global fit* | 1.77E+08 | 5.64E−09 | 7.39E+00 | 28884 |
| 5Afcys | Dimer | Human | 1.09E+06 | 2.17E−03 | 90.5 | global fit* | 5.02E+08 | 1.99E−09 | 4.69E+00 | 57768 |
| 5Afcys | | Cyno | 1.06E+06 | 1.37E−03 | 82.6 | global fit* | 7.70E+08 | 1.30E−09 | 1.88E+01 | 28884 |
| 5Afcys | Monomer | Cyno | 4.43E+05 | 2.56E−03 | 71 | global fit* | 1.73E+08 | 5.77E−09 | 7.29E+00 | 28884 |
| 5Afcys | Dimer | Cyno | 1.12E+06 | 2.44E−03 | 77.7 | global fit* | 4.61E+08 | 2.17E−09 | 6.05E+00 | 57768 |
| 5Afcys | | Rat | 1.23E+06 | 2.82E−03 | 43.4 | global fit* | 4.36E+08 | 2.30E−09 | 1.08E+01 | 28884 |
| 5Afcys | Monomer | Rat | 5.38E+05 | 3.03E−03 | 42.3 | global fit* | 1.77E+08 | 5.63E−09 | 4.74E+00 | 28884 |
| 5Afcys | Dimer | Rat | 1.71E+06 | 3.41E−03 | 48.9 | global fit* | 5.01E+08 | 2.00E−09 | 1.31E+01 | 57768 |

*Data were fit globally over a analyte concentration range of 15.6 to 500 nM

9.3 Comparison of Monomer and Dimer sFv5AF-Cys-sCalcitonin Conjugate Binding to pIgR D6-GST

These experiments used the same capture protocol described above. An anti-GST antibody was immobilized to the Biacore chip surface, and the D6-GST fusion protein was bound to the antibody. The analytes were sFvAF-Cys salmon calcitonin conjugates tested over a concentration range of 15.6 to 500 nM. The conjugates were prepared using the non-clevable mal-sac linker. The conjugate prepared from sFv5A-G4-Cys monomer is designated Az014; the comparable dimer conjugate is designated Az015.

TABLE 13
RESULTS OF BIACORE ASSAY OF SCALCITONIN-SFV
CONJUGATES
1:1 binding model nM.

| Analyte | Ligand | ka (1/Ms) | kd (1/s) | Rmax | conc | KA (1/M) | KD (M) | Chi\hat{ }2 | Expt. |
|---|---|---|---|---|---|---|---|---|---|
| AZ014 | D6 human | 3.87E+04 | 7.26E−03 | 89.1 | global fit* | 5.33E+06 | 1.88E−07 | 1.73 | 1st |
| AZ015 | D6 human | 1.29E+05 | 1.27E−03 | 97.6 | global fit* | 1.01E+08 | 9.89E−09 | 2.4 | 1st |
| AZ014 | D6 cyno | 3.97E+04 | 7.47E−03 | 75.4 | global fit* | 5.31E+06 | 1.88E−07 | 1.23 | 1st |
| AZ015 | D6 cyno | 1.27E+05 | 1.75E−03 | 82.3 | global fit* | 7.25E+07 | 1.38E−08 | 2.9 | 1st |
| AZ014 | D6 rat | 3.42E+04 | 3.91E−03 | 33.5 | global fit* | 8.76E+06 | 1.14E−07 | 0.614 | 1st |
| AZ015 | D6 rat | 1.70E+05 | 2.68E−03 | 47.8 | global fit* | 6.35E+07 | 1.57E−08 | 1.53 | 1st |
| AZ014 | D6 human | 3.65E+04 | 6.97E−03 | 90.5 | global fit* | 5.23E+06 | 1.91E−07 | 1.8 | 2nd |
| AZ015 | D6 human | 1.22E+05 | 1.54E−03 | 102 | global fit* | 7.93E+07 | 1.26E−08 | 2.22 | 2nd |
| AZ014 | D6 cyno | 4.16E+04 | 6.54E−03 | 69.7 | global fit* | 6.36E+06 | 1.57E−07 | 1.35 | 2nd |
| AZ015 | D6 cyno | 1.28E+05 | 1.78E−03 | 85.3 | global fit* | 7.19E+07 | 1.39E−08 | 1.24 | 2nd |
| AZ014 | D6 rat | 3.79E+04 | 2.77E−03 | 29.7 | global fit* | 1.37E+07 | 7.32E−08 | 0.471 | 2nd |
| AZ015 | D6 rat | 1.77E+05 | 2.23E−03 | 46.3 | global fit* | 7.94E+07 | 1.26E−08 | 0.949 | 2nd |

*Data were fitted globally over an analyte concentration range of 15.6 to 500

There is little variation in the K_{D} among different species (human, simian, rat) as reflected in Table 13, and the variation from experiment to experiment is small. When comparing the binding of sFv forms to one species of D6-GST fusion, the dimer conjugate exhibits a higher affinity than the monomer conjugate. The difference is 7 to 19-fold for each species. The difference in K_{D} between the monomer and dimer conjugates for the D6-GST fusion is 7-19-fold, with the dimer conjugate having higher affinity. When comparing the monomer and dimer conjugates, the differences in K_{D} appear to be due to changes in both the association rate constant (ka) and the dissociation rate constant (kd).

In sum, the dimers Calcitonin conjugate has an affinity for the pIgR Domain 6 GST (D6-GST) that is approximately 10-fold higher than the affinity exhibited by the monomer conjugate; the affinity of the monomer and dimer conjugates for the D6-GST constructs do not vary significantly between different species; and the variation between experiments is small, demonstrating the reproducibility of this method.

### Example 10

### Stability of sFv5AF-Cys Monomers and Dimers

10.1. Protease Stability Assays

In order to assess the relative stability of multivalent complexes and compounds, the following experiments were carried out.

The substrates tested were sFv5AF, monoclonal antibody M1, and sFv5AF:M1 complexes. The proteases and extracts that were used were trypsin, chymotrypsin, monkey, rat and human intestinal juices. Trypsin and chymotryp sin were purchased from a commercial vendor (Worthington Biochemical). Samples were taken prior to addition of proteases or extracts (S, starting material), at t=0 min, 15 min, 90 min and 4 hr. The t=0 sample was taken from samples set in ice; subsequent time points were taken during incubation at the specified temperatures. Samples were subject to SDS-PAGE, and the gels were transferred to nitrocellulose and probed with polyclonal anti-sFv5AF antibody, M1 (which is directed to FLAG tag present at the N-terminus of sFv5AF), and HRP conjugated anti-mouse IgG, which detects M1. Incubations were carried out at the specified temperatures and with or without various protease inhibitors.

10.1.1. Room Temperature Incubation

Immunoblotting with anti 5AF showed little degradation over 4 hr. A size shift, which likely represents the loss of the myc 6×His tag from the sFvAF molecule, is seen even in the t=0 samples, indicating the tag is lost during pre-incubation at 4 degrees, during sample heating in SDS sample buffer, and/or during SDS-PAGE.

Immunoblotting with M1 detects the FLAG tag at the N-terminus of sFv5AF. The FLAG tag is lost in the t=0 trypsin samples, but the tag is lost slowly in chymotrypsin and human intestinal juices. This result is consistent with the fact that the FLAG tag contains 2 lysines residues, which makes it a better substrate for trypsin than other enzymes.

10.1.2. Incubations With or Without Protease Inhibitors

Incubations at 37° C. were carried out as described for the room temperature incubations described above. 5AF stability was assayed in the presence of trypsin, chymotrypsin, human intestinal juice, and monkey jejunal juice at room temperature. One set of samples comprised the protease inhibitors chymostatin, leupeptin, and aprotinin, whereas a different set of samples contained no protease inhibitors.

Immunoblotting with anti 5AF detects sFv5AF. Even at 37 degrees, substantial degradation of sFv5AF by chymotrypsin and trypsin is not observed. A size shift (reduction in apparent Mw), suggestive of a loss of the myc 6×HIS tag is seen in all cases except for the t=0 chymotrypsin sample, and in all of the chymotrypsin samples with the protease inhibitors.

Immunoblotting with M1 detects the FLAG tag at the N-terminus of SAF. The FLAG tag is lost in the t=0 trypsin samples, but is more stable in chymotrypsin and intestinal juices.

10.2. Detergent Stability Assays

One hundred and twenty-five (125) μg of sFv5AF-Cys, alone or in the presence of either 0.1% Tween 20 or 0.1% Triton X-100, was subjected to size exclusion chromatography on a 1×44 cm Superdex 75 column in PBS. The relative amounts of monomer and dimer remained unchanged with the detergent treatment.

The results, as represented by the percent area under the peaks, are as follows.

| | | |
|---|---|---|
| sFv5AF-Cys (no detergent) | 47.2% dimer | 38.8% monomer |
| sFv5AF-Cys + 0.1% Tween 20 | 49.8% dimer | 38.2% monomer |
| sFv5AF-Cys + 0.1% Triton X-100 | 49.4% dimer | 37.9% monomer |

### Example 11

### Fusion Proteins Comprising Tandem Single-Chain Antibodies

11.1. Multivalent Single-Chain Antibodies

Reading frames are prepared that encode two or more copies of a single-chain antibody amino acid sequence and are used to prepare a molecule that is a single polypeptide chain that comprises two or more copies of the sFv. The multivalent single-chain antibodies are prepared using recombinant DNA expression systems as described herein. The multivalent single-chain antibodies are noncovalently associated or chemically bonded to a biologically active molecule to produce compounds of the invention.

One skilled in the art will be able to assay a variety of multivalent single-chain antibodies for desirable and undesirable properties in order to identify and produce those that are optimized for particular applications. Optimized multivalent single-chain antibodies are associated with or bonded to a biologically active molecule to produce compounds of the invention.

11.2. Fusion Proteins Comprising Multivalent Single-Chain Antibodies

In instances where the biologically active molecule is a polypeptide, a reading frame that encodes the two or more tandem copies of the sFv and the biologically active polypeptide is prepared. Expression of this reading frame in recombinant DNA expression systems leads to the production of a fusion protein that comprises a biologically active polypeptide and a multivalent sFv in a single polypeptide chain.

It may be appropriate to alter the distance and orientation of the biologically active molecule polypeptide and the two or more V(H) and V(L) regions of the sFv to prepare fusion proteins that are optimized for one or more desirable attributes. Any order or arrangement of elements may be used. By way of non-limiting example, a fusion protein having two tandem repeats of a sFv and a biologically active polypeptide (BAP) could have any of the following structures:

VH1-VL1-BAP-VH2-LV2

VH1-VL1-VH2-VL2-BAP

BAP-VH1-VL1-VH2-LV2

BAP-VH1-VH2-VL2-LV1

VH1-VH2-VL2-VL1-BAP, etc.

Molecules having multiple, for example two, BAP's are also within the scope of the invention:

BAP1-VH1-VL1-BAP2-VH2-VL2

BAP1-VH1-VL1-VH2-VL2-BAP2

VH1-VL1-VH2-VL2-BAP1-BAP2

BAP1-BAP2-VH1-VL1-VH2-VL2, etc.

### Example 12

### Liposomal Formulations

12.1. Structure of Liposomes

Liposomes are microscopic spheres having an aqueous core surrounded by one or more outer layer(s) made up of lipids arranged in a bilayer configuration (see, generally, Chonn et al., Current Op. Biotech., 1995, 6, 698). Liposomes may be used as cellular delivery vehicles for bioactive agents in vitro and in vivo (Mannino et al., Biotechniques, 1988, 6, 682; Blume et al., Biochem. et Diophys. Acta, 1990, 1029, 91; Lappalainen et al., Antiviral Res., 1994, 23, 119. For example, it has been shown that large unilamellar vesicles (LUV), which range in size from about 0.2 to about 0.4 microns, can encapsulate a substantial percentage of an aqueous buffer containing large macromolecules. RNA, DNA and intact virions can be encapsulated within the aqueous interior of liposomes and delivered to brain cells in a biologically active form (Fraley et al., Trends Biochem. Sci., 1981, 6, 77). Liposome-based gene therapy is reviewed by Tseng et al., Pharm. Sci. Tech. Today 1:206-213, 1998; and Ropert, Braz. J. Biol. Res. 32:63-169, 1999. U.S. Pat. No. 5,834,441 is stated to describe liposomes for the delivery of AAV-derived nucleic acids.

Liposomes may be unilamellar (single layer) or multilamellar (multilayer, often compared to an onion skin) and they may be loaded with drugs, peptides, proteins, nucleic acids, carbohydrates, plasmids, vitamins, cosmetics, and the like (Bakker-Woudenberg et al., Liposomes as carriers of antimicrobial agents or immunomodulatory agents in the treatment of infections, Eur J Clin Microbiol Infect Dis 1993;12 Suppl 1:S61-67; Gregoriadis et al., Liposomes in drug delivery. Clinical, diagnostic and ophthalmic potential, Drugs 1993 45:15-28). Examples of techniques for encapsulating molecules into liposomes are described by Mayer et al., Techniques for encapsulating bioactive agents into liposomes, Chem Phys Lipids 40:333-345, 1986.

Liposomes make it possible to encapsulate water soluble and water insoluble substances and avoid the use of other formulations that depend on emulsification and/or surfactants. Liposomes enable the ability to control delivery characteristics of substances with the use of biodegradable and nontoxic materials that comprise the liposome formulation. While substances are contained in the liposome, they are resistant to enzymes and oxidants that exist in the vicinity of the liposome. Liposomes may be injected into a patient; intravenous or subcutaneous injection may be used. In addition, liposomes can be administered to the gastrointestinal tract or the respiratory tract. Liposomes may be encapsulated.

Liposomes are formed from vesicle-forming lipids which generally include one or more neutral or negatively charged phospholipids, typically one or more neutral phospholipids, usually in combination with one or more sterols, particularly cholesterol. Non-limiting examples of lipids useful in liposome production include phosphatidyl compounds, such as phosphatidylglycerol, phosphatidylcholine, phosphatidylserine, sphingolipids, phosphatidylethanolamine, cerebrosides and gangliosides.

Often, the major lipid component of liposomes is a phosphatidylcholine (PC) or PC derivative. PC derivatives with a variety of acyl chain groups of varying chain length and degree of saturation are commercially available or may be synthesized by known techniques. For purposes of filter sterilization, less-saturated PCs are generally more easily sized, particularly when the liposomes must be sized below about 0.3 microns. PCs containing saturated fatty acids with carbon chain lengths in the range of about 14 to about 22 carbon atoms are commonly used particularly diacyl phosphatidylglycerols. Illustrative phospholipids include, for example, dipalmitoylphosphatidylcholine, phosphatidylcholine and distearoylphosphatidylcholine. Phosphatidylcholines with mono- and di-unsaturated fatty acids and mixtures of saturated and unsaturated fatty acids may also be used. Other suitable phospholipids include those with head groups other than choline, such as, for example, ethanolamine, serine, glycerol and inositol. Other suitable lipids include phosphonolipids in which the fatty acids are linked to glycerol via ether linkages rather than ester linkages. In some embodiments, liposomes include a sterol, e.g., cholesterol, at molar ratios of from about 0.1 to about 1.0 (sterol: phospholipid).

12.2. Sterically Stabilized Liposomes

The term “sterically stabilized liposome” refers to a liposome comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids, such as monosialoganglioside GM1, or is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (Allen et al., FEBS Letts., 1987, 223, 42; Wu et al., Cancer Res., 1993, 53, 3765; Papahadjopoulos et al., Ann. N.Y. Acad. Sci., 1987, 507, 64; Gabizon et al., Proc. Natl. Acad. Sci. USA, 1988, 85, 6949; U.S. Pat. No. 4,837,028 and published PCT application WO 88/04924, both to Allen et al. U.S. Pat. No. 5,543,152 to Webb et al.; and published PCT application WO 97/13499 to Lim et al.

Many liposomes comprising lipids derivatized with one or more hydrophilic polymers, and methods of preparation thereof, are known in the art. Sunamoto et al. (Bull. Chem. Soc. Jpn., 1980, 53, 2778) describe liposomes comprising a nonionic detergent. Liposomes comprising phosphatidylethanolamine (PE) derivatized with PEG or PEG stearate or other PEG-derivatized phospholipids, e.g., DSPE-PEG, formed from the combination of distearoylphosphatidylethanolamine (DSPE) and PEG have significant increases in blood circulation half-lives. (Blume et al. Biochimica et Biophysica Acta, 1990, 1029, 91; Klibanov et al., FEBS Letts., 1990, 268, 235). Liposomes having covalently bound PEG moieties on their external surface are described in European Patent No. 0,445,131 BE and WO 90/04384 to Fisher. Liposome compositions containing about 1 to about 20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0,496,813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized via functional surface moieties.

12.3. Targeting of Liposomes

Liposomes can be either passively or actively targeted. Passive targeting utilizes the natural tendency of liposomes to distribute to cells of the reticuloendothelial system in organs that contain sinusoidal capillaries. Active targeting, by contrast, involves modification of the liposome by coupling thereto a specific ligand such as a viral protein coat (Morishita et al., Proc. Natl. Acad. Sci. USA, 1993, 90, 8474), monoclonal antibody (or a suitable binding portion thereof), sugar, glycolipid or protein (or a suitable oligopeptide fragment thereof), or by changing the composition and/or size of the liposome in order to achieve distribution to organs and cell types other than the naturally occurring sites of localization.

Targeting of liposomes may be achieved in a variety of ways. Various linking groups are used to join lipid chains of the liposome to a targeting element. The targeting element binds a specific cell surface molecule found predominantly on cells to which delivery of the compounds of the invention is desired. Targeting elements include, by way of non-limiting for example, a hormone, growth factor or a suitable oligopeptide fragment thereof which is bound by a specific cellular receptor predominantly displayed on by cells to which delivery is desired, or a polyclonal or monoclonal antibody, or a suitable fragment thereof (e.g., Fab; scFv) that specifically binds an antigenic epitope found predominantly on targeted cells.

The targeting of liposomes may be controlled by coating the outside surface of the liposome with targeting agents such as an antibody, F(ab′)2 or Fab fragment of an antibody, cytokines, enzymes, domains and portions of proteins, peptides, polypeptides, carbohydrates, nucleic acids, oligonucleotides, etc. Such coating substances may be present in various amounts on the surface of the liposomes. In the present invention, fusion proteins that project a pIgR ligand from a bi-layer lipid membrane are used to target liposomes.

Targeting of liposomes to different cell types can also be modulated by manipulating the type and ratio of lipids present therein. See, for example, Duzgune et al., Mechanisms and kinetics of liposome-cell interactions, Adv Drug Deliv Rev 1999 40:3-18; Schreier et al., Targeting of liposomes to cells expressing CD4 using glycosylphosphatidylinositol-anchored gp120. Influence of liposome composition on intracellular trafficking. J Biol Chem 1994 269:9090-9098; and Shi et al., Noninvasive gene targeting to the brain, Proc. Natl. Acad. Sci. USA 97:7567-7572, 2000; Shimizu et al., Formulation of liposomes with a soybean-derived sterylglucoside mixture and cholesterol for liver targeting. Biol Pharm Bull 1997 20:881-886.

12.4. Insertion of Transmembrane Proteins into Liposomes

Transmembrane proteins are inserted into the lipid bilayer of liposomes in a variety of ways. Purified proteins can be reconstituted into liposomes via in vitro reconstitution using biochemical methods (Slepushkin et al., Biochem Biophys Res Commun 1996 227:827-33; Brenner et al., Methods Enzymol 2000;322:243-252; Xu et al., J Membr Biol 1999 170:89-102; Genchi et al., Plant Physiol 1999 120:841-848; Lagutina et al., Biochemistry (Mosc) 1998 63:1328-1334; Orellana et al., Mimicking rubella virus particles by using recombinant envelope glycoproteins and liposomes, J Biotechnol 1999 75:209-219).

Some membrane proteins apparently insert into membranes spontaneously, i.e., on their own accord (Mel et al., Biochemistry 1993 32:2082-2089; Antonsson et al., Biochem J 2000 345:271-278). In some instances, spontaneous transmembrane insertion of membrane proteins into liposomes may be facilitated by lecthins, particularly short-chain lecthins (Dencher, Biochemistry 1986 25:1195-1200).

Membrane-spanning and/or membrane-inserting synthetic amino acid sequences can be prepared by in silico design and in vitro experimentation. See, e.g., Wimley et al., Biochemistry 2000 39:4432-4442; Chung et al., Biochemistry 1996;35:11343-11354; Bormann et al., J Biol Chem 1989 264:4033-4037; Percot et al., Design and characterization of anchoring amphiphilic peptides and their interactions with lipid vesicles, Biopolymers 1999 50:647-655; and Chakrabarti et al., Influence of charge, charge distribution, and hydrophobicity on the transport of short model peptides into liposomes in response to transmembrane pH gradients, Biochemistry 1994 33:8479-8485.

Transmembrane and membrane-directing amino acid sequences from such proteins are included in fusion proteins that further comprise a targeting element. Such fusion proteins can be introduced into the membranes of liposomes or of enveloped virons.

As used herein, the term “transmembrane” refers to amino acid sequence that traverse both of the lipid layers of a membrane, as well as “anchored” proteins which comprise a lipophilic moiety that may be incorporated into the outer lipid layer of a membrane. Membrane anchoring structures direct the fusion protein to a liposomal or viral membrane, where it is preferably anchored in the outer lipid layer, projecting into the environment surrounding such membrane-bounded vesicles. For example, it has been demonstrated that mammalian proteins can be linked to myristic acid by an amide-linkage to an N-terminal glycine residue, to a fatty acid or diacylglycerol through an amide- or thioether-linkage of an N-terminal cysteine, respectively, or covalently to a phophotidylinositol (PI) molecule through a C-terminal amino acid of a protein (for a review, see Biochem. J., 244:1-13, 1987). In the latter case the PI molecule is linked to the C-terminus of the protein through an intervening glycan structure, and the PI is incorporated into the phospholipid bilayer; hence the term “GPI” anchor. Specific examples of proteins know to have GPI anchors and their C-terminal amino acid sequences have been reported (Biochemica et Biophysica Acta 988:427-454, 1989; Ann. Rev. Biochem. 57:285-320, 1988). Incorporation of these amino acids or peptide domains into the amino- or carboxy-terminus of a fusion protein can direct the fusion protein to the surface of a liposome or enveloped virion.

12.5. Preparation of Liposomes

Liposomes are prepared by any of a variety of known techniques. For example, liposomes can be formed by any conventional technique for preparing multilamellar lipid vesicles (MLVs), i.e., by depositing one or more selected lipids on the inside wall of a suitable vessel by dissolving the lipid in chloroform, evaporating the chloroform and then adding an aqueous solution which comprises the agent(s) to be encapsulated to the vessel, allowing the aqueous solution to hydrate the lipid, and swirling or vortexing the resulting lipid suspension. This process yields a mixture including the desired liposomes.

As another example, techniques used for producing large unilamellar vesicles (LUVs), such as, e.g., reverse-phase evaporation, infusion procedures and detergent dilution, can be used to produce the liposomes. These and other methods for producing lipid vesicles are described in Liposome Technology, Volume I (Gregoriadis, Ed., CRC Press, Boca Raton, Fla., 1984). The liposomes can be in the form of steroidal lipid vesicles, stable plurilamellar vesicles (SPLVs), monophasic vesicles (MPVs) or lipid matrix carriers (LMCs) of the type disclosed in U.S. Pat. Nos. 4,588,578 and 4,610,868 (both to Fountain et al.), U.S. Pat. No. 4,522,803 (to Lenk et al.), and U.S. Pat. No. 5,008,050 (to Cullis et al.). In the case of MLVs, the liposomes can be subjected to multiple (five or more) freeze-thaw cycles to enhance their trapped volumes and trapping efficiencies and to provide a more uniform interlamellar distribution of solute if desired (Mayer et al., J. Biol. Chem., 1985, 260, 802). Specific methods for making particular oligodeoxynucleotide:liposome compositions are described in U.S. Pat. No. 5,665,710 to Rahman et al.

Following their preparation, liposomes may be sized to achieve a desired size range and relatively narrow distribution of sized particles. In preferred embodiments, the liposomes have a lower range of diameters of from about 50 to about 75 nM, most preferably about 60 nM, and an upper range of diameters from about 75 to about 150 nM, most preferably about 125 nM, where “about” indicates ±10 nM.

Several techniques are available for sizing liposomes to a desired size range. Sonicating a liposome suspension by either bath or probe sonication produces a progressive size reduction down to small unilamellar vesicles (SUVs) less than about 0.05 microns in size. Homogenization, which relies on shearing energy to fragment large liposomes into smaller ones, is another known sizing technique in which MLVs are recirculated through a standard emulsion homogenizer until a selected liposome size range, typically between about 0.1 and about 0.5 microns, is achieved. Extrusion of liposomes through a filter or membrane is another method for producing liposomes having a desired size range (see, for example, U.S. Pat. No. 4,737,323 to Martin et al. and U.S. Pat. No. 5,008,050 to Cullis et al.). Other useful sizing methods are known to those skilled in the art. In most such methods, the particle size distribution can be monitored by conventional laser-beam size determination or other means known in the art.

Liposomes may be dehydrated, preferably under reduced pressure using standard freeze-drying equipment, for extended storage. Whether dehydrated or not, the liposomes and their surrounding media can first be frozen in liquid nitrogen and placed under reduced pressure. Although the addition of the latter freezing step makes for a longer overall dehydration process, there is less damage to the lipid vesicles, and less loss of their internal contents, when the liposomes are frozen before dehydration.

To ensure that a significant portion of the liposomes will endure the dehydration process intact, one or more protective sugars may be made available to interact with the lipid vesicle membranes and keep them intact as water is removed. Appropriate sugars include, but are not limited to, trehalose, maltose, sucrose, lactose, glucose, dextran and the like. In general, disaccharide sugars may work better than monosaccharide sugars, with trehalose and sucrose being particularly effective in most cases, but other, more complicated sugars may alternatively be used. The amount of sugar to be used depends on the type of sugar and the characteristics of the lipid vesicles. Persons skilled in the art can readily test various sugars and concentrations to determine what conditions work best for a particular lipid vesicle preparation (see, generally, Harrigan et al., Chem. Phys. Lipids, 1990, 52, 139, and U.S. Pat. No. 4,880,635 to Janoff et al.). Generally, sugar concentrations of greater than or equal to about 100 mM have been found to result in the desired degree of protection. Once the liposomes have been dehydrated, they can be stored for extended periods of time until they are to be used. The appropriate conditions for storage will depend on the chemical composition of the lipid vesicles and their encapsulated active agent(s). For example, liposomes comprising heat labile agents should be stored under refrigerated conditions so that the potency of the active agent is not lost.

12.6. Pharmaceutical Formulations of Liposomes

Numerous pharmaceutical formulations of liposomes have been developed for delivery to a variety of cell types and tissues have been described. Non-limiting examples include formulations for the intranasal administration of vaccines (U.S. Pat. No. 5,756,104), and aerosol formulations for the delivery of anti-cancer drugs (U.S. Pat. No. 6,090,407). Liposomes may be encapsulated by, and/or incorporated into, formulations such as pills, tablets, capsules, caplets, suppositories, liquids designed for deliver via the alimentary canal, preferably via oral administartion. Pharmaceutical formulations that comprise liposomes and which are used for the delivery of macromolecules, including but not limited to proteins and nucleic acids, are described in, by way of non-limiting example, U.S. Pat. No. 6,132,764, Targeted polymerized liposome diagnostic and treatment agents; U.S. Pat. No. 5,879,713, Targeted delivery via biodegradable polymers; U.S. Pat. No. 5,851,548, Liposomes containing cationic lipids and vitamin D; U.S. Pat. No. 5,759,519, Method for the intracellular delivery of biomolecules using thiocationic lipids; U.S. Pat. No. 5,756,352, Thiocationic lipid-nucleic acid conjugates; U.S. Pat. No. 5,739,271, Thiocationic lipids; U.S. Pat. No. 5,711,964, Method for the intracellular delivery of biomolecules using liposomes containing cationic lipids and vitamin D; and U.S. Pat. No. 5,494,682, Ionically cross-linked polymeric microcapsules.

### Example 13

### Oral Transport

The ability of a composition or compound of the invention to deliver Mab's via the gastrointestinal tract is evaluated as follows.

13.1. Animal Preparation

A cannula is placed in the j ejunum, ileum, or colon of a rat. The end of the cannula is threaded under the skin until it exits the skin between the shoulders of the rat; when located in this manner, the rat cannot damage the cannula and the cannula remains patent for long times. A cannula is also placed in the jugular vein and threaded under the skin so it also exits between the shoulders. A harness may be used to further protect the cannula. The intestinal cannula may be used for administering materials directly into the intestine. The jugular cannula may be used to withdraw blood, which can be further analyzed for the quantity and for biological and biophysical characteristics and functions. The test article (0.2 to 1 ml) is administered through the cannula into the intestine and blood is withdrawn at timed intervals. Heparin is used to keep the jugular vein from clotting.

Alternatively, a urethral catheter, such as C. R. Bard, Inc. Covington, Ga., All-Purpose Urethral Catheter with Funnel End, 16 inches length, two eyes, X-ray opaque rubber, is used to administer the test article to the colon. In this case, a rat is anesthetized with Ketamine. The catheter is cut so that about 8 cm of catheter remains. A Luer lock fitting is placed on the cut end of the catheter and a 1 ml syringe containing the test article is attached to the Luer lock fitting. The catheter is filled with the test article. A mark at 7.5 from the tip of the catheter is made with ink and the catheter is inserted into the rectum of the rat until the mark is just visible. The syringe is then used to deliver the required volume of test article, generally 0.2 to 1 ml in volume.

13.2. Assays

The test article may be detected by radioiodinating or otherwise radiolabeling it. One or more components of a conjugate protein may be labeled. Blood that is collected is then used to determine the number of cpm in a measured weight or volume of blood. The test article may be detected by an appropriate biochemical or biological assay, including without limitation ELISA, enzyme, receptor binding, etc. An ELISA using the antigen for the Mab in the test article is typically used.

The biophysical features of the test article may be detected by immunoprecipitation, by SDS-PAGE and detection of the radiolabel by various imaging processes including autoradiography, by Western blotting with agents that bind to the test article, by gel sizing on Sephadex or Sepharose resins of an appropriate size, etc.

13.3. Pharmacokinetics

A graph of the amount of test article present in blood as a function of time allows one to observe the amount of transport over time. Dividing the amount of test article in blood by the amount of test article administered to the rat yields the percent absorbed dose. By administering the same amount of test article through the intestine and through an intravenous injection and comparing the area under the curve, the absolute bioavailability is determined. The bioavailabilty relative to other routes of injection, such as subcutaneous, may also be obtained. Those skilled in the art will know how to perform the pharmacokinetic analysis.

The transport of the test article may be compared to controls of the Mab that has not been conjugated to a targeting element. Such comparison demonstrates the specificity and selectivity of transport.

### Example 14

### Assays for the in vivo Delivery of pIgR-Targeted Fusion Proteins

A variety of assays are used to determine the extent of delivery of a monoclonal antibody from the lumen of an organ to the body of an animal. Non-limiting examples of such organs are the gastrointestinal tract and the lung. For example, in order to determine the delivery Mab′ s from the gastrointestinal tract is determined according to the following procedures.

14.1. Animal Preparation

A cannula is implanted into the jugular vein of a rat for the purpose of collecting blood samples at various times. Another cannula is implanted into a region of the intestine, jejunum, ileum, or colon, for the purpose of administering the therapeutic entity to the intestine. A 350-375 gram Spraque-Dawley rat is suitable for this purpose although other strains of rats may be used. The cannulae are guided under the skin so that they exit the skin directly between the shoulders of the rat. This position prevents the rat from damaging the cannulae. A single rat per cage is required. The fusion protein is administered to the rat 2 to 7 days after the cannulae are implanted. During this time, the rat is observed for its general health and to determine the patency of the cannulae.

14.2. Administration of Test Article and Sample Collection

The test article (i.e., Mab-comprising composition or compound of the invention) is given to the rat through the intestinal cannula. Before administration, a sample of blood (approximately 200 microliters) is withdrawn through the jugular vein cannula. Samples of blood are collected over a 8 to 48 hour period. The jugular cannula is kept patent by using saline with a small amount of heparin to prevent clotting. The blood is collected into a 1.5 ml Eppendorf tube that contains 5 microliters of heparin (about 5 to 50 units/ml) to prevent clotting. The blood is kept on ice for up to 1 hour, but no longer, before it is centrifuged in a table top Eppendorf centrifuge for 30 to 60 seconds. The supernatant is collected (plasma) and stored in a suitable manner, usually by freezing at −80° C. Blood may also be collected and allowed to clot and form clotted material, which is then separated from the serum by centrifugation. The serum is stored in a suitable manner, usually by freezing at −80° C.

14.3. Assays

The presence and amount of the Mab is measured using any appropriate assay. For example, the Mab may be radioiodinated using ¹²⁵I using any of the usual methods of radioiodination that are known to those skilled in the art. These methods include using chloramine-T, immobilized chloramine-T, iodine monochloride, lactoperoxidase beads, or Iodogen. Radioiodinated Mab's are separated from unreacted ¹²⁵I by chromatography, including size separation on Sephadex or Sepharose, or by dialysis. The weight of the blood is determined by collecting the blood into a preweighed Eppendorf or small glass tube and determining the weight of the blood by subtraction after weighing the tube containing the blood. The entire tube may be counted in a gamma counter and the number of counts per minute divided by the weight of the blood to determine the number of cpm per gram of blood (essentially equivalent to the cpm/ml of blood). A graph of the cpm/ml of blood as a function of time after administration of the radiolabelled therapeutic entity is used to illustrate the transport of the Mab from the intestine into blood.

The test article may be examined to determine if it has the same molecular weight by SDS-PAGE. A sample of the plasma may be compared on SDS-PAGE with a sample of the radiolabelled test article that was administered through the cannula. If the patterns of radioactivity (autoradiography) are the same, then it is concluded that the Mab that is present in blood is not degraded. The blood sample is reacted to immunoprecipitate the therapeutic entity. The immunoprecipitated sample is compared to an immunoprecipitated sample from the stock radiolabelled fusion protein by separation and visualization on SDS-PAGE. A quantitative estimate of the amount of Mab is made by comparing the amount of cpm that was immunoprecipitated from blood samples and from stock radiolabelled fusion protein.

An immunoassay such as, for example, an enzyme linked immunosorbent assay (ELISA) is used to determine the concentration of the test article. In this case, the test article is not radiolabelled. An antibody that recognizes an epitope present in the Mab, or the antigen to which the Mab is directed, is coated to the bottom of 96-well plates. After washing, the presence and quantity of bound Mab is determined by binding to the immobilized Mab a second antibody that is conjugated to a detectable enzyme such as, e.g., horse radish peroxidase or alkaline phosphatase. After washing, a substrate for horse radish peroxidase or alkaline phosphatase is incubated in the well. Substrate is detectable or results in a detectable product. The amount of the product determined by spectrophotometry at an appropriate wavelength. A control curve (using known quantities of the fusion protein) is used to determine the concentration of the Mab in the plasma samples.

14.4. Related Protocols

Similar experiments are conducted to examine the ability of compositions and components of the invention for the rectal delivery of Mab's via, e.g., a suppository. In these experiments, a composition or compound of the invention is administered by a rectal tube. A catheter is inserted through the anus of an anesthetized rat. The urinary catheter inserted 7.5 cm through the anus will result in delivery within the colon.

Similarly, the above described procedures can be used to examine a Mab's delivery via inhalation. In these experiments, the fusion protein is administered as an aerosol or microparticulate formulation to the nasal or pulmonary cavity.

### Example 15

### In vivo Testing

Rat cancer models are used to determine the efficacy of compositions and compounds of the invention (for an example of the application of such methods, see Beneditti et al., Cancer Res. 59:645-652, 1999). For example, a pIgR-targeted Mab that reacts with epidermal growth factor receptor (EGFR) is tested for its ability to inhibit the growth of tumors implanted into a rat. If the Mab reacts with rat EGFR, the tumor cells that are implanted are of rat origin and grown in a wild type rat. If the Mab reacts with human EGFR (e.g., Cetuximab, ABX-EGF), the tumors cells that are implanted are of human origin and are grown in an immune compromised (scid) rat.

15.1. Animal Preparation

The rat is prepared for administration of the therapeutic entity by inserting a cannula into a region of the intestine, such as the jejunum, ileum, or colon. After the surgery required to insert the cannula, the rat is optionally allowed to rest for 2 to 7 days to recover. During this time, the rat is observed for its general health and the patency of the cannula. During this time, or shortly before the surgery, tumor cells are injected subcutaneously into the flank of the rat. Depending on the specific tumor cell line used and its ability to form tumors, 10,000 to 5,000,000 cells are injected subcutaneously. The cells are first grown in tissue culture medium and then taken up as a suspension. The cells are injected into the animal subcutaneously.

The tumor cells are allowed to grow for 5 to 14 days before the tumor is treated with the test article administered through an intestinal cannula in a formulation appropriate for the gastrointestinal tract. Alternatively, formulations for the inhalation delivery of proteins are tested via administered through the pulmonary or nasal cavity using an aerosol. The EGFR-expressing cell line TE8, an esophageal squamous cell carcinoma, and the EGFR-deficient cell line H69 may be used to determine the efficacy of the test article. (Suwa et al., International Journal of Cancer. 75:626-634, 1998). The A431 cell line, a human epidermoid carcinoma tumor cell line, may also be used to test the effects of the test article. The A431 cells are grown in athymic rodents, including rats. Athymic nude rats bearing orthotopically implanted LNCaP tumors may be implanted subcutaneously and treated with the test article. (Rubenstein et al., Medical Oncology 14:131-136, 1997).

Tumor cells, such as C6 cells, may also be implanted stereotactically into the right caudate nucleus of Wistar rats. A cannula into the intestine may also be put into these rats for the purpose of administering the fusion protein. Rats with well-established cerebral C6 glioma foci may be given the fusion protein through the intestinal cannula.

15.2. Assays

Measurements of the tumor size are made using calipers to measure the dimensions of the tumor in two directions. The volume of the tumor is determined by multiplying the longest dimension times the square of the shortest dimension and dividing the product by 2. By plotting the tumor volume as a function of time (using the average or mean tumor volume) for a group of rats given the fusion protein, and comparing the same plot for a group of untreated rats bearing a tumor prepared in the same manner, one skilled in the art can determine the ability of the test article to inhibit or slow the growth of the tumor and preferably, to eradicate the tumor.

The mean survival time of tumor bearing rats is about 15-20 days in this model. The efficacy of the test article may be measured by comparing the life span of control rats (i.e, tumor bearing rats given no test article) to rats given the test article (Pu et al., Journal of Neurosurgery 92:132-139, 2000).

### Example 16

### Pharmaceutical Formulations of Compositions and Compounds

16.1. Capsules, Tablets, Caplets, Etc.

A preferred pharmaceutical formulation of a composition or compound of the invention is a pill, e.g., a capsule, tablet, caplet or the like, that is suitable for oral administration. Numerous capsule manufacturing, filling, and sealing systems are well-known in the art. Preferred capsule dosage forms can be prepared from gelatin and starch. Gelatin has been the traditional material, and the dosage forms are generally produced by well known dip molding techniques. After manufacture, gelatin capsules are filled with the desired composition and then sealed. A more recently developed alternative to gelatin dosage forms are capsules produced from starch. Starch capsules (typically made from potato starch) afford several advantages compared to gelatin capsules, including pH-independent dissolution, better suitability for enteric coating, water in the dosage form is tightly bound to the starch (and is thus less likely to migrate into the composition encapsulated in the dosage form), and the absence of animal-derived ingredients (which may be antigenic or contaminated with pathogens). Vilivilam, et al., PSTT 3:64-69, 2000). Starch capsules are odorless and rigid, and exhibit similar dissolution properties as compared to gelatin capsules.

Capsules of any suitable size can be manufactured. Starch capsules are typically made in two pieces, a cap and a body, using injection molding techniques. See Eith et al., Manuf. Chem. 58: 21-25, 1987; Idrissi et al., Pharm. Acta. Helv. 66: 246-252, 1991; Eith et al., Drug Dev. Ind. Pharm. 12: 2113-2126, 1986. The two pieces are then sealed together during filling to prevent separation. Sealing can achieved by applying a hydroalcoholic solution to the inner surface of the cap.

16.2. Enteric Coatings

After making the capsule dosage forms, if desired, they can be coated with one or more suitable materials. For example, when it is desired to deliver the encapsulated composition to the intestines, one or enteric coatings may be applied. Traditionally, enteric coatings were used to prevent gastric irritation, nausea, or to prevent the active ingredient from being destroyed by acid or gastric enzymes. However, these coatings can also be used to deliver agents to particular gastrointestinal regions.

A variety of enteric coatings are known in the art, and any suitable coating, or combinations of coatings, may be employed. Suitable coatings for starch capsules include aqueous dispersions of methacrylic acid copolymers and water-based reconstituted dispersion of cellulose acetate phthalate (CAP). See Brogmann et al., Pharm. Res. 11:S-167; Vilivalam, et al., Pharm. Res. 14:S-659, 1999; Vilivalam et al., Pharm. Res. 15:S-645, 1998; Bums et al., Int. J. Pharm. 134: 223-230, 1996; Davis et al., Eur. J. Nucl. Med., 19: 971-986, 1992. Indeed, a variety of coatings can be used to coat encapsulated dosage forms. These coatings include pH-sensitive materials, redox-sensitive materials, and materials that can be broken down by specific enzymes or microorganisms present in the intestine. Watts et al. (1995), WIPO publication WO35100, reports an enteric-coated starch capsule system for targeting sites in the colon. The pH sensitive enteric coating begins to dissolve when the dosage form enters the small intestine, and coating thickness dictates in which region of the intestine the capsule disintegrates, for example, in the terminal ileum or in the ascending, transverse, or descending colon. Other coatings, or combinations of coatings, can also be used to achieve the same effect.

16.3. Packaging

After a dosage from is prepared, it is typically packaged in a suitable material. For pill or tablet dosage forms, the dosage forms may be packaged individually or bottled en masse. An example of individual packaging PVC-PVdC-Alu, where aluminum blisters are covered with PVC (polyvinyl chloride) coated with PVdC (polyvinylidene chloride) to improve water vapor and oxygen protection. Suitable bottling materials include tinted, transluscent, or opaque high density polyethylene.

Those skilled in the art will be able to use the preceding information to prepare appropriate formulations for the gastrointestinal delivery of the fusion proteins of the invention. Other related information is known in the art and may be utilized to prepare appropriate formulations for gastrointestinal delivery of the fusion proteins.

### Example 17

### Formulations and Medical Devices for Inhalation Therapy

One aspect of the invention relates to an aerosol inhaler, or other medical device, for delivery of a monoclonal antibody. Such devices are useful for inhalation therapies based on the compositions and compounds of the invention. The term “inhalation therapy” refers to the delivery of a therapeutic agent, such as a drug or a fusion protein of the invention, in an aerosol form to the respiratory tract (i.e, pulmonary delivery). For reviews, see Gonda (J. Pharm. 89:940-945, 2000); Byron et al. (J. Aerosol Med. 7:49-75, 1994; and Niven (Crit. Rev. Ther. Drug Carrier Syst. 12:151-231, 1995).

The compositions and compounds of the invention are formulated for pulmonary delivery, and incorporated into medical devices such as inhalers, according to the following considerations and criteria, as well as other considerations and criteria known to those skilled in the art. A practicioner of the art will be able to use the following information to prepare appropriate formulations and medical devices for pulmonary delivery of the compositions and compounds of the invention.

17.1. Inhalation Therapy Using Monoclonal Antibodies

Inhalers comprising compositions and compounds the invention may be used to deliver them quickly, and via self-administration. Such medical devices can be used to treat chronic or acute disorders or disease where it is desired to deliver Mab's via an inhalation route and in a short period of time. Chronic attacks of a disorder or disease include, for example, asthma attacks. A non-limiting example of Mab′ s useful for treating asthma is CDP 835. Other Mab's that may desirably be delivered via inhalation include without limitation BEC2, ABX-EGF, E25, Palivixumab, and the like.

17.2. Formulations for Inhalation Therapy

Compositions and compounds that are intended to be used in inhalation therapy must be formulated into a composition that is appropriate for delivery via inhalation. Two formulations of therapeutic agents that are useful for inhalation therapy include those in the form of liquid particles and solid particles. The liquid formulations are generated by nebulizing solutions of the therapeutic agent. Solid particle formulations are either in the form of a powder suspended in a propellant which is administered from a metered dose inhaler, or simply as a powder that is administered from a dry powder inhaler. In the case of polypeptide therapeutic agents, solid particle aerosols can be made by lyophilizing the polypeptide from solution and then milling or grinding the lyophilized drug to the desired particle size for pulmonary administration.

Non-limiting examples of formulations of therapeutic agents, including proteins, for inhalation therapy are described in Bittner et al. (J. Microencapsul. 16:325-341, 1999; Flament et al. (Int. J. Pharm. 178:101-109, 1999); and Langenback et al. (Pediatr. Pulmonol. 27:124-129, 1999), and references cited therein. Non-limiting examples of inhalation formulations of proteins are described in U.S. Pat. Nos. 5,230,884; 5,354,562; 5,457,044; 5,888,477; 5,952,008; 5,970,973; 6,000,574; 6,051,551; 6,060,069; 6,085,753; and 6,121,247.

17.3. Aerosol Inhalers

An “aerosol inhaler” or “inhaler” is a device by which a patient can actively breathe in a given dose of a therapeutic agent. A typical application for such a medical device is for the treatment of an acute asthma attack. Delivery of drugs via inhalation, however, can be used for many other treatments including those described herein. For example, drugs administered by inhalation may be taken up by cells lining the interior of the pulmonary system and be delivered into the body therefrom. In the present invention, fusion proteins that comprise a biologically active polypeptide and an appropriate pIgR targeting polypeptide and, as a result of reverse transcytosis, will be delivered into the circulatory system of a patient.

Inhalers have long been used to deliver drugs into a patient's lungs. Typically, an inhaler provides a mixture of therapeutic agents and air or some other type of propellant gas. The formulation of the therapeutic agent is delivered into the patient when he or she inhales from a mouthpiece on the inhaler. In general aerosol delivery systems rely on a mixture of the therapeutic agent with one or more propellants, and optional inactive ingredients, to increase dispersion and stability of the therapeutic agent. Inhalation of the formulation can be by either the nose or mouth and often is self-administered. Because of the small volume of each dosage, the propellant generally evaporates simultaneously or shortly after delivery of the therapeutic agent.

Correct inhalation of an aerosol formulation may require good hand-breath coordination. In the case of some inhalers, delivery ideally proceeds in such a manner that a patient first exhales and then applies the device to his mouth and as he begins to inhale, triggers the action of the inhaler by activating an actuating element thereof. Upon such activation, the aerosol formulation consisting of a propellant and therapeutic agent present in the said propellant and distributed therein, passes from the inhaler through a nozzle into the respiratory system of the patient. Inhalation of the therapeutic formulation into the respiratory system can be via the nasal cavity, the bucal cavity, or both. As the patient actively inhales gases from these cavities the aerosol formulation is delivered to the lungs. Atomization and dispersion of the therapeutic formulation in an inhaler can be triggered electronically or mechanically.

In general, there are three types of inhalers that are used to deliver therapeutic agents during inhalation therapy: nebulizers, metered dose inhalers (MDIs) and dry powder inhalers (DPIs). Each of these types of inhaler may be used to deliver the fusion proteins of the invention.

Nebulizers are electrical devices that send a therapeutic composition directly into a patient's mouth by tube or, in children, by clear mask. Nebulizers require no hand-breath coordination. The prescribed amount of medicine is placed in the device, a tube in inserted into the mouth (or, in the case of children, a mask is placed the child's nose and mouth), and breathing commences normally until the therapeutic composition is depleted.

Measured-dose inhalers (MDIs, a.k.a. metered dose inhaler) send a measured dose of a therapeutic composition into the mouth using a small amount of pressurized gas. In MDIs, a “spacer” may be placed between the drug reservoir and the mouth to control the amount inhaled in a single application. The therapeutic composition into the spacer, which is then squeezed by the patient as he quickly inhales the composition. MDIs have recently fallen out of favor because the common MDI propellant chlorofluorocarbon (CFC) has been found to deplete the atmosphere's ozone layer, and there are international agreements to phase out the production and use of CFC.

Dry-powder inhalers (DPIs) provide a popular alternative to aerosol-based inhalers. DPIs have the advantage of not requiring a propellant. However, because they have no propellant, PDIs depend on the force of inhalation to get the therapeutic composition into the lungs. Children, people with severe asthma, and people suffering acute attacks may be unable to produce enough airflow to use dry-powder inhalers successfully. Nonetheless, DPIs are used in inhalation therapies involving the fusion proteins of the invention.

Various types of inhalers for delivering therapeutic agents are known. By way of non-limiting examples, see U.S. Pat. Nos. 3,938,516; 4,627,432; 5,941,240; 6,116,239; 6,119,688; and 6,119,684. One example of a dry powder inhaler that is within the scope of the invention is the Diskhaler, which is described in U.S. Pat. No. 4,627,432. The Spiros inhaler, described in U.S. Pat. No. 5,921,237, is another dry powder inhaler that is within the scope of the invention. Other dry powder inhalers that are within the scope of the invention include but are not limited to those described in U.S. Pat. Nos. 6,012,454; 6,045,828; 6,055,980; 6,056,169; 6,116,237; and 6,116,238.

Those skilled in the art will be able to use the preceding information to prepare appropriate formulations and medical devices for pulmonary delivery of the molecules of the invention. Other necessary information is known in the art and may be utilized to prepare appropriate formulations and medical devices.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Sequence Listing

1. A multimeric molecular complex comprising at least 2 compounds, each compound comprising at least one targeting element directed to a ligand that confers transcytotic or paracellular transporting properties to a molecular complex specifically bound to said ligand.

2. The multimeric molecular complex of claim 1, wherein one or more of said properties of said complex are enhanced as compared to a second compound having no more than 1 targeting element.

3. A first multimeric molecular complex comprising n targeting elements directed to a ligand that confers transcytotic or paracellular transporting properties to a compound bound to said ligand, wherein one or more of said properties of said first multimeric molecular complex are enhanced as compared to a second multimeric molecular complex having m targeting elements, wherein n and m are both whole integers, and n>m.

4. The multimeric molecular complex of claim 1, wherein at least 2 of said at least 2 compounds are complexed by non-covalent interactions.

5. The multimeric molecular complex of claim 1, wherein at least 2 of said at least 2 compounds are complexed by covalent bonds.

6. The multimeric molecular complex of claim 3, wherein at least one of said targeting elements in said multimeric molecular complex is substantially the same as at least one other targeting element in said multimeric molecular complex.

7. The multimeric molecular complex of claim 3, wherein at least one of said targeting elements in said multimeric molecular complex is substantially the same as at least one other targeting element of said second multimeric molecular complex.

8. The multimeric molecular complex of claim 3, wherein at least one of said targeting elements in said multimeric compound is substantially the same as at least one other targeting element in said multimeric molecular complex, and wherein said targeting element is substantially the same as the targeting element of said second molecular complex.

9. The multimeric molecular complex of claim 3, wherein said one or more enhanced properties are selected from the group consisting of endocytotic properties, transcytotic properties, exocytotic properties, and paracellular transporting properties.

10. The multimeric molecular complex of claim 3, wherein said one or more enhanced properties is an increase in the relative rate of a process selected from the group consisting of endocytosis, transcytosis, exocytosis, and paracellular transport, or a preference for apical to basolateral transcytosis.

11. A compound comprising at least 2 targeting element directed to a ligand that confers transcytotic or paracellular transporting properties to a compound specifically bound to said ligand.

12. The compound of claim 11, wherein one or more of said properties of said compound are enhanced as compared to a second compound having no more than 1 targeting element.

13. A compound comprising n targeting elements directed to a ligand that confers transcytotic or paracellular transporting properties to a compound bound to said ligand, wherein one or more of said properties of said compound are enhanced as compared to a second compound having m targeting elements, wherein n and m are both whole integers, and n>m.

14. The compound of claim 13, wherein at least one of said targeting elements in said compound is substantially the same as at least one other targeting element in said compound.

15. The compound of claim 13, wherein at least one of said targeting elements in said compound is substantially the same as at least one other targeting element of said second compound.

16. The compound of claim 13, wherein at least one of said targeting elements in said compound is substantially the same as at least one other targeting element in said compound, and wherein said targeting element is substantially the same as the targeting element of said second molecular complex.

17. The compound of claim 13, wherein said one or more enhanced properties are selected from the group consisting of endocytotic properties, transcytotic properties, exocytotic properties, and paracellular transporting properties.

18. The compound of claim 13, wherein said one or more enhanced properties is an increase in the relative rate of a process selected from the group consisting of endocytosis, transcytosis, exocytosis, and paracellular transport, or a preference for apical to basolateral transcytosis.

19. The compound of claim 13, wherein said ligand is selected from the group consisting of the polyimmunoglobulin receptor, the secretary component of pIgR and the stalk of pIgR.

20. A protein conjugate comprising a biologically active calcitonin polypeptide having a chemical linkage to at least one targeting element directed to a ligand that confers transcytotic or paracellular transporting properties to a molecular complex specifically bound to said ligand.

21. The protein conjugate of claim 21, wherein said protein conjugate is capable of undergoing apical to basolateral transcytosis.

22. The compound of claim 13, wherein said ligand is selected from the group consisting of the amino acid sequences LRKED, QLFVNEE, LNQLT, YWCKW, GWYWC, STLVPL, SYRTD, KRSSK; and regions R1, R2a, R2b, R3a, R3b, R3c, R4a, R4b, R4c, R4d, R5a, R5b, R5c, R5d, R5e, R6a, R6b, R6c, R6d, R6e, R6f, R7a, R7b, R7c, R7d, R7e, R7f, R7g, R8a, R8b, R8c, R8d, R8e, R8f, R8g, and R8h.

23. The compound of claim 13, wherein said compound further comprises a biologically active moiety.

24. The compound of claim 23, wherein said biologically active moiety is selected from the group consisting of a small molecule, a nucleic acid and a polypeptide.

25. The compound of claim 23, wherein said biologically active moiety and said at least 2 targeting elements are polypeptides.

26. The compound of claim 25, wherein said compound is a fusion protein comprising said biologically active moiety and said at least 2 targeting elements.

27. The compound of claim 25, wherein said compound is a protein conjugate comprising said biologically active moiety and said at least 2 targeting elements are polypeptides.

28. The compound of claim 23, wherein said biologically active moiety is a targeting element directed to a second ligand.

29. The compound of claim 23, wherein said biologically active moiety is an antibody or derivative thereof.

30. A pharmaceutical composition comprising the compound of claim 13.

31. A method of delivering a biologically active agent to an animal in need thereof, comprising contacting said animal with the compound of claim 23.

32. A method for transporting a biologically active agent through an epithelial barrier, comprising contacting said epithelial barrier with the compound of claim 23.

33. A method of treating a disease in an animal, comprising contacting said animal with the compound of claim 23.

34. The method of claim 33, wherein said disease is selected from the group consisting of colitis; ulcerative colitis; diverticulitis; Crohn's disease; gastroenteritis; inflammatory bowel disease; bowel surgery ulceration of the duodenum, a mucosal villous disease including but not limited to coeliac disease, past infective villous atrophy and short gut syndromes; an apoptosis mediated disease; an inflammatory disease; an autoimmune disease; a proliferative disorder; an infectious disease; a degenerative disease; a necrotic disease, asthma; allergic bronchopulmonary aspergillosis; hypersensitivity pneumonia, eosinophilic pneumonia; emphysema; bronchitis; allergic bronchitis bronchiectasis; cystic fibrosis; hypersensitivity pneumotitis; occupational asthma; sarcoid, reactive airway disease syndrome, interstitial lung disease, hyper-eosinophilic syndrome, parasitic lung disease; lung cancer and diabetes, consisting of rheumatoid arthritis, multiple sclerosis, graft-versus-host disease, diabetes mellitus, sarcoidosis, granulomatous colitis, systemic lupus erythematosus and osteoarthritis, pancreatitis, asthma, adult respiratory distress syndrome, glomeralonephritis, rheumatoid arthritis, systemic lupus erythematosus, scleroderma, chronic thyroiditis, Grave's disease, autoimmune gastritis, insulin-dependent diabetes mellitus (Type I), autoimmune hemolytic anemia, autoimmune neutropenia, thrombocytopenia, chronic active hepatitis, myasthenia gravis, psoriasis, graft vs. host disease, osteoporosis, multiple myeloma-related bone disorder, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia, myocardial ischemia, spinal muscular atrophy, multiple sclerosis, AIDS-related encephalitis, HIV-related encephalitis, aging, alopecia, neurological damage due to stroke in a patient Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and ophthalmoplegia.

35. The compound of claim 13, wherein said compound further comprises a detectable moiety.

36. A method of identifying a disease in an animal, comprising contacting said animal with the compound of claim 34.

37. A method of treating a disease in an animal, comprising contacting said animal with the compound of claim 20.

38. The method of claim 37, wherein said disease is selected from the group consisting of osteoporosis, osteogenesis imperfecta, Paget's disease, hypercalcemia, vasospasms, ischemia, renal failure, and male impotence.
